(19) United States
(12) Patent Application Publication
(10) Pub. No.: US 2026/0356902 A1
(43) Pub. Date: Nov. 12, 2026

(54) COMPOSITIONS AND METHODS OF USE OF IL-10 AGENTS IN CONJUNCTION WITH CHIMERIC ANTIGEN RECEPTOR CELL THERAPY

(71) Applicant: Eli Lilly and Company, Indianapolis, IN (US)

(72) Inventors: Scott Alan MCCAULEY, Brisbane, CA (US); Martin OFT, Palo Alto, CA (US)

(21) Appl. No.: 19/452,833

(22) Filed: Jan. 19, 2026

Related U.S. Application Data
(63) Continuation of application No. 15/733,970, filed on Jun. 12, 2019, now abandoned.
(60) Provisional application No. 62/687,103, filed on Jun. 19, 2018.

(51) Int. Cl. C07K 14/54; A61K 38/00; A61K 40/11; A61K 40/31; A61K 40/42; A61K 45/06; A61K 47/60; C07K 14/705; C12N 5/0783; C12N 15/86

(52) U.S. Cl. CPC C07K 14/5428; A61K 40/11; A61K 40/31; A61K 40/4211; A61K 45/06; A61K 47/60; C07K 14/705; C12N 5/0636; C12N 15/86; A61K 38/00; A61K 2239/48; C07K 2319/02; C12N 2740/15043

(57) ABSTRACT

The present invention relates to methods of modulating the activity of CAR-T cells in the treatment of diseases, disorders and conditions by the administration of an IL-10 agent. The invention further provides engineered CAR-T cells to express additional therapeutically effective agents. The present invention further provides improved pharmaceutical and therapeutic compositions and methods relating to the use of CAR-T cell therapies in the treatment of disease in mammalian subjects.

### CROSS-RELATED APPLICATIONS

This application is a continuation of U.S. patent application Ser. No. 15/733,970 filed Dec. 1, 2020, which is the US National Stage of International Application No. PCT/US2019/036711 filed Jun. 12, 2019 which claims the benefit of U.S. Provisional Application No. 62/687,103 filed Jun. 19, 2018. The foregoing applications are incorporated herein by reference.

### SEQUENCE LISTING

The instant application contains a Sequence Listing which has been submitted electronically in XML file format and is hereby incorporated by reference in its entirety. Said XML copy, created on Jan. 19, 2026, is named X22084A_44P54WOUS2_SL.xml and is 178,167 bytes in size.

### FIELD OF THE INVENTION

This invention relates to methods of using IL-10 agents in combination chimeric antigen receptor cell therapy to modulate immune responses in the treatment or prevention of diseases, disorders and conditions. In particular, the present disclosure describes the use of IL-10 agents in conjunction with chimeric antigen receptor-T cell (CAR-T cell) therapy.

### INTRODUCTION

Interleukin-10 (IL-10) is a pleiotropic cytokine that regulates multiple immune responses through actions on T-cells, B cells, macrophages, and antigen presenting cells (APCS). As a result of its pleiotropic activity, IL-10 has been linked to a broad range of diseases, disorders and conditions, including inflammatory conditions, immune-related disorders, fibrotic disorders, metabolic disorders and cancer. Clinical and pre-clinical evaluations with IL-10 for a number of such diseases, disorders and conditions have demonstrated its therapeutic potential in a variety of human therapeutic applications. A variety of IL-10 derivatives, variants and analogs, both naturally occurring and synthetic, have been produced which retain IL-10 activity. Human IL-10 (hIL-10) is a homodimer of two IL-10 polypeptides with each monomer comprising 178 amino acids, the first 18 of which comprise a signal peptide which is excised during cellular expression and does not form part of the mature IL-10 molecule. The IL-10 polypeptides are non-covalently associated to form the dimeric IL-10 molecule. In particular, pegylated forms of IL-10 have been shown to possess improved activity, prolonged half-life and utility in certain therapeutic settings.

CAR-T cell therapy represents an emerging therapy for cancer, particularly in the treatment of B and T-cell lymphomas. CAR-T cell therapy comprises the use of adoptive cell transfer (ACT), a process which employs a subject's own T-cells which are modified using recombinant DNA techniques to express synthetic T-cell receptor (“TCR”) termed a chimeric antigen receptor (or “CAR”) alter the innate tropism of the T-cell so as to direct the engineered T-cell bind to a target cell. A CAR is typically an engineered fusion polyprotein which provides a synthetic T-cell receptor such that when the CAR contacts the ligand to which it is engineered to interact, the CAR-T-cell becomes activated. The chimeric antigen receptor is typically a single polypeptide comprising multiple functional domains, typically a targeting ectodomain that is expressed on the outer surface of a T-cell transformed with ane expression vector encoding the CAR. The CAR further comprises a transmembrane domain that spans the cell membrane and an intracytoplasmic endodomain which mediates chemical reactions that provide intracellular signaling upon binding of the ectodomain to its target.

For example, the ectodomain of the CAR may be specific for a known antigen present on a target cell. Frequently, the CAR is engineered to bind to a marker expressed on the surface of a neoplastic cell.

In the typical practice of CAR-T cell therapy, T-cells are isolated from a subject by apherisis and genetically altered to express CARs by transfecting the isolated T-cells ex vivo with a recombinant vector encoding a CAR resulting in a population of recombinantly modified CAR-T cells. CAR-T cells are often generated using patient-derived memory CD8+ T-cells recombinantly modified to express the CAR. Following ex vivo amplification, the CAR-T cells are typically infused back into the patient where the CAR-T cells circulate until the ectodomain of the CAR encounters its target binding ligand resulting in selective immune response to the target cell population.

As discussed further hereafter, CAR-T cell therapy has, in part, been limited by both the induction of antigen-specific toxicities by the CAR-T cells targeting normal tissues expressing the target-antigen and the extreme potency of CAR-T cell treatments. These toxicities have been observed to result in life-threatening cytokine-release syndromes. In particular, it has been observed that high affinity T-cell receptor interactions with significant antigen burden can lead to activation-induced cell death. The present invention provides compositions and methods that provides enhanced activity of the engineered CAR-T cells facilitating the use of lower dosages of CAR-T cells thereby minimizing adverse events associated with CAR-T cell therapy.

### SUMMARY OF THE INVENTION

The present disclosure contemplates compositions and methods of using CAR-T cell therapy in conjunction with an IL-10 agent to modulate a T-cell-mediated immune response to a target cell population in a subject.

In certain embodiments of the present disclosure, the disclosure provides a method of modulating a T-cell-mediated immune response to a target cell population in a subject, the method comprising:
(a) obtaining a plurality of T-cells from a subject;
(b) contacting the isolated plurality of T-cells with a recombinant vector, the recombinant vector comprising a nucleic acid sequence encoding a chimeric antigen receptor (CAR) operably linked to an expression control sequence functional in the T-cell, the contacting being conditions permitting uptake of the nucleic acid sequence by the plurality of T-cells;
(c) isolating those T-cells from the plurality of T-cells contacted with the nthe recombinant vector that express the nucleic acid sequence encoding the chimeric antigen receptor (CAR-T cells);
(d) administering to the subject a therapeutic amount of the isolated CAR-T cells of step (c) in combination with a therapeutically effective amount of an IL-10 agent.

In certain embodiments of the present disclosure, the disclosure provides a method of modulating a T-cell-mediated immune response to a target cell population in a subject, the method comprising administering in combination to the subject:
(a) a therapeutically effective amount of CAR T-cells expressing a CAR, the antigen recognition domain of which is capable of binding to the target cell population; and
(b) a therapeutically effective amount of an IL-10 agent.

In certain embodiments, the present disclosure provides a method of treating a subject suffering from a disease, disorder or condition with a therapeutically effective amount of an IL-10 agent, wherein the IL-10 agent is administered to the subject prior to, simultaneously with, or subsequent to administration of a therapeutically effective amount of CAR-T cells, the antigen recognition domain of the CAR of the CAR-T cells being capable of binding to a cell surface molecule of a target population of cells characteristic of the disease, disorder or condition.

In certain embodiments, the present disclosure provides a method of treating a subject suffering from a disease, disorder or condition the method comprising the administration of a therapeutically effective amount of CAR-T cells, the antigen recognition domain of the CARs of the CAR-T cells being capable of binding to a cell surface molecule of a target population of cells characteristic of the disease, disorder or condition, the method comprising the steps of: (a) contacting the CAR-T cells with IL-10 agent ex vivo for a period of time, and (b) administering a therapeutically effective amount of the CAR-T cells of step (a) to the subject.

In certain embodiments, the present disclosure provides a method of treating a subject suffering from a disease, disorder or condition the method comprising the administration of a therapeutically effective amount of CAR-T cells, the antigen recognition domain of the CARs of the CAR-T cells being capable of binding to a cell surface molecule of a target population of cells characteristic of the disease, disorder or condition, the method comprising the steps of: (a) contacting the CAR-T cells with IL-10 agent ex vivo for a period of time, and (b) administering a therapeutically effective amount of the CAR-T cells of step (a) to the subject in combination with an IL-10 agent (the IL-10 agent administered to the subject being either the same or different than the IL-10 agent used to treat the CAR-T cells prior to administration).

In certain embodiments, the present disclosure provides a method of enhancing the cytoxic activity of a population of CAR-T cells wherein the CAR-T cells are contacted with an IL-10 agent ex vivo.

In certain embodiments, the present disclosure provides a method of enhancing the immunomodulatory activity of a population of CAR-T cells wherein the CAR-T cells are contacted with an IL-10 agent ex vivo.

In certain embodiments, the present disclosure provides a method of treating a subject suffering from a disease, disorder or condition wherein with CAR-T cell therapy wherein the CAR-T cells are treated ex vivo with an IL-10 agent prior to their administration to a subject.

In certain embodiments, the present disclosure provides a method of treating a subject suffering from a disease, disorder or condition wherein with CAR-T cell therapy wherein the CAR-T cells are treated ex vivo with an IL-10 agent prior to their administration to a subject followed by the administration of the IL-10 treated CAR-T cells to the subject in combination with an IL-10 agent (the IL-10 agent administered to the subject being either the same or different than the IL-10 agent used to treat the CAR-T cells prior to administration).

In certain embodiments, the present disclosure provides a method of treating a subject suffering from a disease, disorder or condition with CAR-T cell therapy wherein the CAR-T cells are treated ex vivo with an IL-10 agent prior to their administration to a subject wherein the CAR-T cells are transfected with a recombinant vector encoding a CAR and an IL-10 agent, wherein the vector-encoded IL-10 agent is either the same or different than the IL-10 agent used to treat the cells ex vivo prior to administration.

In certain embodiments, the present disclosure provides a method of treating a subject suffering from a disease, disorder or condition with CAR-T cell therapy, wherein the CAR comprises an antigen specific domain (ASD) which specifically recognizes and binds to a cancer antigen present on a neoplastic cell.

In certain embodiments, the present disclosure provides a method of treating a subject suffering from a disease, disorder or condition with CAR-T cell therapy in combination with the administration of an IL-10 agent wherein the IL-10 agent enhances the function of activated memory CD8+ T-cells.

In certain embodiments, the present disclosure provides a method of treating a subject suffering from a disease, disorder or condition with CAR-T cell therapy in combination with the administration of an IL-10 agent wherein the IL-10 agent is administered to the subject in an amount sufficient to enhance cytotoxic function of the CAR-T cells.

In certain embodiments, the present disclosure provides a method of treating a subject suffering from a disease, disorder or condition with CAR-T cell therapy in combination with the administration of an IL-10 agent wherein the IL-10 agent is administered to the subject sufficient to maintain an IL-10 serum trough concentration of at least 1 ng/ml over a period of time.

In certain embodiments, the present disclosure provides a method of treating a subject suffering from a disease, disorder or condition with CAR-T cell therapy in combination with the administration of an IL-10 agent wherein the IL-10 agent is administered to the subject subcutaneously.

In certain embodiments, the present disclosure provides a method of treating a subject suffering from a disease, disorder or condition with CAR-T cell therapy in combination with the administration of an IL-10 agent wherein the IL-10 agent is administered to the subject for the treatment or prevention of a disease, disorder or condition (e.g., a cancer-related disorder) in a subject in conjunction with the introduction to the subject of cells genetically modified to express an IL-10 agent.

In certain embodiments, the present disclosure provides a method of treating a subject suffering from a disease, disorder or condition with CAR-T cell therapy in combination with the administration of an IL-10 agent wherein the administering modulates a T-cell-mediated immune response to a target cell population in a subject, comprising introducing to the subject a therapeutically effective plurality of cells genetically modified to express a) a chimeric antigen receptor, wherein the chimeric antigen receptor comprises at least one antigen-specific targeting region capable of binding to the target cell population; and b) a therapeutically effective amount of an IL-10 agent.

In some embodiments where the CAR-T cell is modified to express the IL-10 agent, the chimeric antigen receptor and the IL-10 agent are expressed by the same vector, while in other embodiments the chimeric antigen receptor and the IL-10 agent are expressed by different vectors.

In particular embodiments, the therapeutically effective plurality of cells is transfected with a vector that expresses the IL-10 agent in a therapeutically effective amount wherein the therapeutically effective amount is an amount sufficient to enhance cytotoxic function of the CAR-T cell. The vector may be, for example, a plasmid or a viral vector. In particular embodiments, expression of the IL-10 agent is modulated by an expression control element. In particular embodiments, expression of the IL-10 agent is modulated by an expression control element to maintain the serum trough concentration of the IL-10 agent at or above approximately 0.1 ng/ml, 0.5 ng/ml, 1 ng/ml, 1.5 ng/ml, 2 ng/ml, 3 ng/ml, 5 ng/ml, or the EC50 of the IL-10 agent.

In particular embodiments, the plurality of cells is obtained from the subject and genetically modified ex vivo. The plurality of cells may be obtained from the subject by apheresis. In some embodiments, the plurality of cells is memory CD8+ T-cells. In some embodiments, the plurality of cells comprises subject derived CD8+ T-cells. In some embodiments the cells are not derived from the subject to be administered.

In certain embodiments, the present disclosure provides a method of treating a subject suffering from a disease, disorder or condition with CAR-T cell therapy in combination with the administration of an IL-10 agent the method comprising introducing to the subject a) a therapeutically effective first plurality of cells genetically modified to express a chimeric antigen receptor, wherein the chimeric antigen receptor comprises at least one antigen-specific targeting region capable of binding to the target cell population; and b) a second plurality of cells genetically modified to express, and optionally secrete, a therapeutically effective amount of an IL-10 agent. In particular embodiments, the second therapeutically effective plurality of cells is transfected with a vector that expresses the IL-10 agent in an amount sufficient to enhance cytotoxic function of the CAR-T cells. In particular embodiment, the therapeutically effective second plurality of cells comprises patient derived CD8+ T-cells transfected with a vector that expresses the IL-10 agent.

In particular embodiments, the first plurality of cells is obtained from the subject and genetically modified ex vivo, while in other embodiments the second plurality of cells is obtained from the subject and genetically modified ex vivo. The present disclosure contemplates embodiments wherein the first plurality of cells and the second plurality of cells are obtained from the subject by an aphaeretic process. In some embodiments, the first plurality of cells is memory CD8+ T-cells, and the second plurality of cells is naïve CD8+ T-cells. In some embodiments, the first plurality of cells and the second plurality of cells are autologous tumor cells.

The present disclosure also contemplates the use of CAR-T cell therapy for the treatment or prevention of a disease, disorder or condition (e.g., a cancer-related disorder) in a subject in combination with the administration of an IL-10 agent (e.g., PEG-IL-10) or the introduction of a vector that expresses an IL-10 agent.

A particular embodiment comprises methods of treating a subject having a cancer-related disease, disorder or condition (e.g., a tumor), comprising a) introducing to the subject a therapeutically effective plurality of cells genetically modified to express a chimeric antigen receptor, wherein the chimeric antigen receptor comprises at least one antigen-specific domain capable of binding specifically to an antigen present on the surface of a target cell of a target cell population; and b) administering to the subject a therapeutically effective amount of an IL-10 agent.

In certain embodiments of the present disclosure, such methods are used in therapeutic protocols for the prevention of a cancer-related disease, disorder or condition in a subject, while in other embodiments such methods are used in therapeutic protocols for the prevention of immune-related disorders. Further aspects of the above-described methods, including dosing parameters and regimens for the IL-10 agents as well as exemplary types of such agents, are described elsewhere herein.

In certain embodiments, the present disclosure provides recombinant vectors comprising a nucleic acid sequence encoding a CAR and an IL-10 agent and method of use thereof. In some embodiments, the CAR is directed to a tumor antigen and the IL-10 agent is hIL-10. In some embodiments, the vector comprises a first nucleic acid sequence encoding a CAR and a second nucleic acid sequence encoding an IL-10 agent, wherein the first and second nucleic acid sequences are operably linked to a first and second expression control element respectively, the first and second expression control elements being the same or different.

In certain embodiments, the present disclosure provides recombinant vectors comprising a nucleic acid sequence encoding a CAR and an IL-10 agent, the vector comprising a polycistronic nucleid acid comprising a first nucleic acid sequence encoding a CAR and a second nucleic acid sequence encoding an IL-10 agent, wherein the polycistronic nucleic acid sequences is operably linked to an expression control element, the polycistronic nucleic acid optionally providing an intervening sequence that enhances expression of the second nucleic acid sequence (e.g. an IRES or FMVD2A sequence). In certain embodiments, the vector is a viral vector. In certain embodiments, the viral vector is a lentiviral vector.

In certain embodiments, the present disclosure provides recombinant vectors comprising a nucleic acid sequence encoding a CAR and an IL-7 agent and methods of use thereof.

In certain embodiments, the present disclosure provides recombinant vectors comprising a nucleic acid sequence encoding a CAR and an IL-12 agent and methods of use thereof.

In certain embodiments, the present disclosure provides recombinant vectors comprising a nucleic acid sequence encoding a CAR and an IL-15 agent and methods of use thereof.

In certain embodiments, the present disclosure provides recombinant vectors comprising a nucleic acid sequence encoding a CAR and an IL-18 agent and methods of use thereof.

In certain embodiments, the present disclosure provides recombinant vectors comprising a nucleic acid sequence encoding a CAR and ITIM inhibitory agent and methods of use thereof.

In certain embodiments, the present disclosure provides recombinant vectors comprising a nucleic acid sequence encoding a CAR and an IL-7 receptor and methods of use thereof.

In certain embodiments, the present disclosure provides recombinant vectors comprising a nucleic acid sequence encoding a CAR and an IL-10 receptor and methods of use thereof.

In certain embodiments, the present disclosure provides recombinant vectors comprising a nucleic acid sequence encoding a CAR and an IL-12 receptor and methods of use thereof.

In certain embodiments, the present disclosure provides recombinant vectors comprising a nucleic acid sequence encoding a CAR and an IL-15 receptor and methods of use thereof.

In certain embodiments, the present disclosure provides recombinant vectors comprising a nucleic acid sequence encoding a CAR and an IL-18 receptor and methods of use thereof.

Additional embodiments of the present disclosure contemplate methods of treating a subject having a cancer-related disease, disorder or condition, comprising introducing to the subject a therapeutically effective plurality of cells genetically modified to express a) a chimeric antigen receptor, wherein the chimeric antigen receptor comprises at least one antigen-specific targeting region capable of binding to the target cell population, and b) an IL-10 agent. In some embodiments, the chimeric antigen receptor and the IL-10 agent are expressed by the same vector, while in other embodiments the chimeric antigen receptor and the IL-10 agent are expressed by different vectors.

In particular embodiments, the therapeutically effective plurality of cells is transfected with a vector that expresses the IL-10 agent in an amount sufficient to enhance cytotoxic function of a T-cell. The vector may be, for example, a non-viral or a viral vector. The present disclosure also contemplates the use of any other means of expressing the IL-10 agent. In particular embodiments, expression of the IL-10 agent is modulated by an expression control element. In particular embodiments, the expression control element is a regulatable promoter. In particular embodiments, the expression control element is tissue specific promoter.

In the embodiments described above, the plurality of cells may be obtained from the subject and genetically modified ex vivo. According to some embodiments of the present disclosure, the plurality of cells is obtained from the subject by an aphaeretic process at treated with at least one IL-10 agent following expansion and for a period of time prior to administration, the period of time being less than about 48 hours, less than about 36 hours, less than about 24 hours, less than about 18 hours, less than about 12 hours, less than about 6 hours, less than about 4 hours, less than about 2 hours, or less than about 1 hour prior to administration to the subject. The plurality of cells comprises memory CD8+ T-cells in particular embodiments and comprises autologous tumor cells in other embodiments.

Still further embodiments of the present disclosure contemplate methods of treating a subject having a cancer-related disease, disorder or condition, comprising introducing to the subject a) a therapeutically effective first plurality of cells genetically modified to express a chimeric antigen receptor, wherein the chimeric antigen receptor comprises at least one antigen-specific targeting region capable of binding to the target cell population, and b) a therapeutically effective second plurality of cells genetically modified to express an IL-10 agent.

In certain embodiments, the methods described above are used in therapeutic protocols for the prevention of a disease, disorder or condition, including a cancer- or an immune-related disease, disorder or condition in a subject.

In particular embodiments, the therapeutically effective first plurality of cells is transfected with a vector that expresses the IL-10 agent in an amount sufficient to enhance cytotoxic function. The therapeutically effective second plurality of cells comprises CD8+ T-cells transfected with a vector that expresses the IL-10 agent in still other embodiments.

In particular embodiments, the first plurality of cells is obtained from the subject and genetically modified ex vivo, while in other embodiments the second plurality of cells is obtained from the subject and genetically modified ex vivo. The present disclosure contemplates embodiments wherein the first plurality of cells and the second plurality of cells are obtained from the subject by an aphaeretic process. In some embodiments, the first plurality of cells is memory CD8+ T-cells, and the second plurality of cells is naïve CD8+ T-cells. The first plurality of cells and the second plurality of cells are autologous tumor cells in still other embodiments.

In each of the aforementioned embodiments, the target cell population may comprise a tumor antigen, examples of which are described elsewhere herein.

The present disclosure contemplates nucleic acid molecules that encode the IL-10 agents described herein. In certain embodiments, the nucleic acid molecule encoding the IL-10 agent(s) is operably linked to an expression control element that confers expression of the nucleic acid molecule encoding the IL-10 agent in a cell transformed with the DNA molecule. In some embodiments, a vector (e.g., a plasmid or a viral vector) comprises the nucleic acid molecule. Also contemplated herein are transformed or host cells that express the IL-10 agent.

The present disclosure contemplates the use of the foregoing agents and methods in combination with additional therapeutic modalities, including but not limited to the administration of additional chemotherapeutic agents, immunomodulatory molecules including immune checkpoint modulators, cytokine agents, cytokine variant agents, cytokine analog agents and modified cytokine agents specifically including fusion proteins of such cytokine agents and PEGylated forms thereof.

In one embodiment, the invention provides a method of treating a mammalian subject suffering from a neoplastic disease the method comprising:
a. obtaining a sample of T-cells derived from the patient;
b. transducing a fraction of T-cells in the sample with a vector, the vector comprising a nucleic acid sequence encoding a chimeric antigen receptor (CAR) the nucleic acid sequence being in operable association with one or more control elements to effect transcription and translation of the nucleic acid sequence encoding a chimeric antigen receptor (CAR) in a T-cell, so as to generate a population of T-cells expressing the CAR;
c. isolating the T-cells expressing the CAR (CAR-T cells);
d. culturing the CAR-T cells ex vivo in the presence of an IL-10 agent; and
e. administering the CAR-T cells from step (d) to the mammalian subject.

In one embodiment, the invention provides the further step of (f) administering to the subject a pharmaceutical formulation comprising a therapeutically effective amount of an IL-10 agent. In one embodiment, the IL-10 agent of step (d) and the IL-10 agent of the pharmaceutical formulation of step (f) are the same IL-10 agent. In one embodiment, the IL-10 agent of step (d) and the IL-10 agent of the pharmaceutical formulation of step (f) are different IL-10 agents. In one embodiment, IL-10 agent of step (d) is rhIL-10 and the pharmaceutical formulation of IL-10 agent of step (f) comprises a PEGylated IL-10 agent. In one embodiment, the pharmaceutical formulation comprises a mono-PEGylated IL-10 agent. In one embodiment, the pharmaceutical formulation comprises a mixture of a mono-PEGylated IL-10 agent and a diPEGylated IL-10 agent. In one embodiment, the administering of a pharmaceutical formulation comprising the IL-10 agent is sufficient to maintain a serum trough concentration of the IL-10 agent in the subject of at least 0.01 ng/ml over a period of at least 72 hours, alternatively at least 0.05 ng/ml over a period of at least 72 hours, alternatively at least 0.1 ng/ml over a period of at least 72 hours, alternatively at least 0.5 ng/ml over a period of at least 72 hours.

In one embodiment, the disclosure provides a method of modulating a T-cell-mediated immune response to a target cell population in a subject, comprising:
a) introducing to the subject a therapeutically effective plurality of cells genetically modified to express a chimeric antigen receptor (CAR), wherein the chimeric antigen receptor comprises at least one antigen-specific targeting region capable of binding to the target cell population; and
b) administering to the subject a therapeutically effective amount of an IL-10 agent wherein the administration of the IL-10 agents results in a serum trough level of at least 0.01 ng/ml. In some embodiments, the IL-10 agent is a mono-PEGylated IL-10 agent or a mixture of a mono-PEGylated IL-10 agent and a diPEGylated IL-10 agent. In some embodiments, the administering of the IL-10 agent to the subject is sufficient to maintain a serum trough concentration of the IL-10 agent in the subject of at least 0.03 ng/ml, alternatively at least 0.06 ng/ml, alternatively at least 0.1 ng/ml, alternatively at least 0.5 ng/ml, alternatively at least 1 ng/ml, alternatively at least 2 ng/ml, or alternatively at least 5 ng/ml over a period of at least 72 hours.

The disclosure further provides a method of modulating a T-cell-mediated immune response to a target cell population in a subject, comprising introducing to the subject a therapeutically effective plurality of cells genetically modified to express:
a) a chimeric antigen receptor (CAR), wherein the chimeric antigen receptor comprises at least one antigen-specific targeting region capable of binding to the target cell population, and
b) an IL-10 agent,
   thereby modulating the T-cell-mediated immune response.

In another embodiment, the disclosure provides a method of modulating a T-cell-mediated immune response to a target cell population in a subject, comprising introducing to the subject:
a) a therapeutically effective first plurality of cells genetically modified to express a chimeric antigen receptor (CAR), wherein the chimeric antigen receptor comprises at least one antigen-specific targeting region capable of binding to the target cell population; and
b) a therapeutically effective second plurality of cells genetically modified to express an IL-10 agent.

In some embodiments, the expression of the IL-10 agent by genetically modified cell provide a local IL-10 agent concentration in the target cell microenvironment of at least 0.005 ng/ml, alternatively at least 0.01 ng/ml, alternatively at least 0.05 ng/ml, alternatively at least 0.1 ng/ml, alternatively at least 0.2 ng/ml, alternatively at least 0.5 ng/ml, alternatively at least 1 ng/ml, or alternatively at least 2 ng/ml.

In another embodiment, the disclosure provides a method of inhibiting apoptosis in a CAR-T cell by contacting the T cell with an effective amount of an IL-10 agent. In some embodiments, the method is practiced ex vivo and the amount of an IL-10 agent is provided in a buffered solution having a concentration of the IL-10 agent of greater than about 0.005 ng/ml, alternatively at least 0.01 ng/ml, alternatively at least 0.05 ng/ml, alternatively at least 0.1 ng/ml, alternatively at least 0.2 ng/ml, alternatively at least 0.5 ng/ml, alternatively at least 1 ng/ml, or alternatively at least 2 ng/ml. In some embodiments, the method is practiced in vivo in a subject and the amount of an IL-10 agent administered to the subject is sufficient to maintain a serum trough concentration of the IL-10 agent in the subject of at least 0.03 ng/ml, alternatively at least 0.06 ng/ml, alternatively at least 0.1 ng/ml, alternatively at least 0.5 ng/ml, alternatively at least 1 ng/ml, alternatively at least 2 ng/ml, or alternatively at least 5 ng/ml over a period of at least 24 hours.

In some embodiments, the CAR-T cell employed provides an antigen recognition domain (ARD) wherein the ARD of the CAR is a polypeptide that specifically binds to HER2, MUC1, telomerase, PSA, CEA, VEGF, VEGF-R2, T1, CD19, CD20, CD22, ROR1, mesothelin, CD33/IL3Ra, c-Met, PSMA, Glycolipid F77, FAP, EGFRvIII, GD-2, NY-ESO-1 TCR, MAGE A3 TCR, 5T4, WT1, KG2D ligand, folate receptor (FRa), platelet-derived growth factor receptor A, or Wnt1 antigens. In some embodiments, the antigen recognition domain of the CAR is selected from the group consisting of an anti-CD19 scFv, an anti-PSA scFv, an anti-CD19 scFv, an anti-HER2 scFv, an anti-CEA scFv, an anti-EGFR scFv, an anti-MUC1 scFv, an anti-HER2-neu scFv, an anti-VEGF-R2 scFv, an anti-T1 scFv, an anti-CD22 scFv, an anti-ROR1 scFv, an anti-mesothelin scFv, an anti-CD33/IL3Ra scFv, an anti-c-Met scFv, an anti-PSMA scFv, an anti-Glycolipid F77 scFv, an anti-FAP scFv, an anti-EGFRvIII scFv, an anti-GD-2 scFv, an anti-NY-ESO-1 scFv, an anti-MAGE scFv, an anti-A3 scFv, an anti-5T4 scFv, an anti-WT1 scFv, or an anti-Wnt1 scFv.

In some embodiments, the CAR-T cell employed as described herein provides an intracellular signaling domain comprising an amino acid sequence derived from the cytoplasmic domain of CD27, CD28, CD137 CD278, CD134, FcεR1γ and β chains, MB1 (Igα) chain, B29 (Igβ) chain, the human CD3 zeta chain, CD3, a syk family tyrosine kinase, a src family tyrosine kinase, CD2, CD5 or CD28. In one embodiment, for CAR-T cell used in the practice of the method provides an intracellular signaling domain comprising an amino acid sequence derived from the cytoplasmic domain of CD28, CD137 (4-1BB), CD134 (OX40), Dap10, CD27, CD2, CD5, ICAM-1, LFA-1 (CD 11a/CD18), Lck, TNFR-I, TNFR-II, Fas, CD30, and CD40. The foregoing method may be combined with the administration to the subject of one or more supplemental agents including chemotherapeutic agents, immune checkpoint modulators, IL-2 agents, IL-7 agents, IL-12 agents, IL-15 agents and IL-18 agents, in particular where the immune checkpoint modulators selected from the group consisting of PD1 modulators, PDL1 modulators, CTLA4 modulators, LAG-3 modulators, TIM-3 modulators, ICOS modulators, OX40 modulators, cd-27 modulators, CD-137 modulators, HVEM modulators, CD28 modulators, CD226 modulators, GITR modulators, BTLA modulators, A2A modulators, IDO modulators and VISTA modulators.

In one embodiment, the disclosure provides a recombinant vector comprising nucleic acid sequences encoding an IL-10 agent, a CAR, and a cytokine the nucleic acid sequences operably linked to an expression control sequence. In some embodiments, the recombinant vector encodes the a polypeptide that specifically binds to HER2, MUC1, telomerase, PSA, CEA, VEGF, VEGF-R2, T1, CD19, CD20, CD22, ROR1, mesothelin, CD33/IL3Ra, c-Met, PSMA, Glycolipid F77, FAP, EGFRvIII, GD-2, NY-ESO-1 TCR, MAGE A3 TCR, 5T4, WT1, KG2D ligand, folate receptor (FRa), platelet-derived growth factor receptor A, or Wnt1 antigens, in particular where the antigen recognition domain of the CAR is selected from the group consisting of an anti-CD19 scFv, an anti-PSA scFv, an anti-CD19 scFv, an anti-HER2 scFv, an anti-CEA scFv, an anti-EGFR scFv, an anti-MUC1 scFv, an anti-HER2-neu scFv, an anti-VEGF-R2 scFv, an anti-T1 scFv, an anti-CD22 scFv, an anti-ROR1 scFv, an anti-mesothelin scFv, an anti-CD33/IL3Ra scFv, an anti-c-Met scFv, an anti-PSMA scFv, an anti-Glycolipid F77 scFv, an anti-FAP scFv, an anti-EGFRvIII scFv, an anti-GD-2 scFv, an anti-NY-ESO-1 scFv, an anti-MAGE scFv, an anti-A3 scFv, an anti-5T4 scFv, an anti-WT1 scFv, or an anti-Wnt1 scFv. In other embodiments, the recombinant vector encodes a CAR wherein the intracellular signaling domain of the CAR comprises an amino acid sequence derived from the cytoplasmic domain of CD27, CD28, CD137 CD278, CD134, FcεR1ζ and β chains, MB1 (Igα) chain, B29 (Igβ) chain, the human CD3 zeta chain, CD3, a syk family tyrosine kinase, a src family tyrosine kinase, CD2, CD5 or CD28, and optionally or in addition a polypeptide comprising an amino acid sequence derived from one or more co-stimulatory domains derived from the intracellular signaling domains of CD28, CD137 (4-1BB), CD134 (OX40), Dap10, CD27, CD2, CD5, ICAM-1, LFA-1 (CD 11a/CD18), Lck, TNFR-I, TNFR-II, Fas, CD30, and CD40. In some embodiments, the cytokine encoded by the vector is selected from the group consisting of IL-7, IL-12, IL-15, and IL18, and variants thereof. In some embodiments, the vector is a viral vector including a lentiviral vector.

The disclosure further provides modified T-cells transformed with the foregoing vectors.

The disclosure further provides a pharmaceutical formulation comprising a CAR-T cell and an IL-10 agent, including where the IL-10 agent is pegylated.

Other embodiments will be apparent to the skilled artisan based on the teachings of the present disclosure. While the present disclosure is generally described in the context of using CAR-T cell therapy for the treatment of cancer, it is to be understood that such therapy is not so limited.

### DETAILED DESCRIPTION OF THE INVENTION

### A. General Interpretation and Construction

Before the present disclosure is further described, it is to be understood that the disclosure is not limited to the particular embodiments set forth herein, and it is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements or the use of a “negative” limitation.

Unless indicated otherwise, parts are parts by weight, molecular weight is weight average molecular weight, temperature is in degrees Celsius (°C), and pressure is at or near atmospheric. Standard abbreviations are used, including the following: bp=base pair(s); kb=kilobase(s); pl=picoliter(s); s or sec=second(s); min=minute(s); h or hr=hour(s); aa=amino acid(s); kb=kilobase(s); nt=nucleotide(s); pg=picogram; ng=nanogram; μg=microgram; mg=milligram; g=gram; kg=kilogram; dl or dL=deciliter; μl or μL=microliter; ml or mL=milliliter; l or L=liter; μM=micromolar; mM=millimolar; M=molar; kDa=kilodalton; i.m.=intramuscular(ly); i.p.=intraperitoneal(ly); SC or SQ=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; PCR=polymerase chain reaction; NHS=N-Hydroxysuccinimide; HSA=human serum albumin; MSA=mouse serum albumin; DMEM=Dulbeco's Modification of Eagle's Medium; GC=genome copy; EDTA=ethylenediaminetetraacetic acid.

Standard methods in molecular biology are described in the scientific literature (See, e.g., Sambrook and Russell (2001) Molecular Cloning, 3rd ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)). The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (See, e.g., Coligan, et al. (2000) Current Protocols in Protein Science, Vols. 1-2, John Wiley and Sons, Inc., NY).

It will be appreciated that throughout this disclosure reference is made to amino acids according to the single letter or three letter codes. For the reader's convenience, the single and three letter amino acid codes are provided below:

TABLE 1
Amino Acid Abbreviations

| | | |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Unless otherwise indicated, the following terms are intended to have the meaning set forth below. Other terms are defined elsewhere throughout the specification. Unless defined otherwise, technical and scientific terms used herein shall be construed as having the same meaning as commonly understood by one of ordinary skill in the art to which this invention belongs.

### B. Definitions

Activity: As used herein, the term “activity” is used with respect to a molecule to describe a property of the molecule with respect to a system (e.g. a test system or biological function such as the degree of binding of the molecule to another molecule, the catalytic activity of a biological agent, the ability to regulate gene expression or cell signaling, differentiation, or maturation, the ability to modulate immunological activity such as immune response, and the like. “Activity” may be expressed as catalytic activity (katal), binding activity (mol-/L), specific activity, e.g., [catalytic activity]/[mg protein], or [immunological activity]/[mg protein], international units (IU), placque forming units (pfu), concentration in a biological compartment, or the like. The term “proliferative activity” encompasses an activity that enhances, promotes, that is necessary for, or that is specifically associated with, for example, cell division, as well as dysregulated cell division as observed in neoplastic diseases, fibrosis, dysplasia, cell transformation, metastasis, and angiogenesis.

Administer/Administration: The terms “administration” and “administer” are used interchangeably herein to refer the act of contacting a subject, including contacting in vitro, in vivo or ex vivo a cell, tissue, organ, or biological fluid of the subject with an agent (e.g. an IL-10 agent, a CAR-T cell, a chemotherapeutic agent, an antibody, checkpoint pathways modulator or a pharmaceutical formulation comprising the foregoing). Administration of an agent may be achieved through any of a variety of art recognized methods including but not limited to the topical, intravenous (including intravenous infusion), intradermal, subcutaneous, intramuscular, intraperitoneal, intracranial, intratumoral, transdermal, transmucosal, intralymphatic, intragastric, intraprostatic, intravascular (including intravenous and intraaterial), intravesical (e.g., the bladder), iontophoretic, pulmonary, intraocular, intraabdominal, intralesional intraovarian, intracerebral, intracerebroventricular injection (ICVI), and the like. The term “administration” includes contact of an agent to a cell, as well as contact of an agent to a fluid, where the fluid is in contact with the cell.

Adverse Event: As used herein, the term “adverse event” refers to any undesirable experience associated with the use of a therapeutic agent or treatment modalilty in a patient. Adverse events do not have to be caused by the administered agent. Adverse events may be mild, moderate, or severe. The classification of adverse events as used herein with respect to the treatment of neoplastic disease is in accordance with the Common Terminology Criteria for Adverse Events v5.0 (CTCAE) dated Nov. 27, 2017 published by the United States Department of Health and Human services, National Institutes of Health National Cancer Institute.

Affinity: The term “affinity” as used herein refers to the degree of specific binding of a molecule (e.g., a TCR, a CAR, an ARD, or antibody) to its target and is measured by the binding kinetics expressed as K_{d}, a ratio of the dissociation constant between the molecule and the its target (K_{off}) and the association constant between the molecule and its target (Kₒₙ). As used herein, the term “high affinity” is used in reference to molecules having a K_{d}<10⁻⁷. Preferred CARs of the invention have a K_{d} for a target antigen 1 of about 100 pM or less at 25 °C. More preferred CARs of the invention have a binding affinity for a tumor antigen of about 10 pM or less at 25 °C.

Agent: As used herein the term “agent” refers to a molecule (e.g. small molecule or polypeptide) or therapeutic modality (e.g. external beam radiation and internal radiation therapy) having identificable characteristics and exhibiting biological or chemical activity in vitro or in vivo.

Agonist: As used herein, the terms “agonist” or “activator” are used interchangeably herein to refer a molecule that interacts with a target to promote, enhance, facilitate or cause an increase in the activity of the target or effects associated with the binding of a ligand to the target. Non-limiting examples of the action of an agonist or activator may include increasing the transcription and/or translation of a nucleic acid sequence, increasing the activity of an enzyme, increasing the kinetics or energetic of the binding of an antibody to its target, the binding of a TCR to its target, or the binding of a CAR to its target.

Antagonist: As used herein, the terms “antagonist” or “inhibitor” are used interchangeably herein to refer to a molecule that decreases, blocks, prevents, delays activation, inactivates, desensitizes, or down-regulates, e.g., a gene, protein, ligand, receptor, biological pathway including an immune checkpoint pathway. In one aspect, an antagonist prevents, reduces, inhibits, or neutralizes the activity of an agonist. In another respect, an antagonist prevents, inhibits, or reduces the activity of a target, e.g., a target receptor, even where there is no identified agonist.

Antibody: As used herein, the term “antibody” refers collectively to: (a) glycosylated and non-glycosylated the immunoglobulins (including but not limited to mammalian immunoglobulin classes IgG1, IgG2, IgG3 and IgG4) that specifically binds to target molecule and (b) immunoglobulin derivatives including but not limited to IgG(1-4)deltaC_{H}2, F(ab′)₂, Fab, ScFv, VH, VL, tetrabodies, triabodies, diabodies, dsFv, F(ab′)₃, scFv-Fc and (scFv)₂ that competes with the immunoglobulin from which it was derived for binding to the target molecule. The term antibody is not restricted to immunoglobulins derived from any particular mammalian species and includes murine, human, equine, camels, antibodies, human antibodies. The term “antibody” encompasses naturally occurring antibodies isolatable from natural sources and as well as engineered antibodies including monoclonal antibodies, bispecific antibodies, chimeric antibodies, humanized antibodies, human antibodies, CDR-grafted, veneered, or deimmunized (e.g., to remove T-cell epitopes) antibodies. The term “antibody” should not be construed as limited to any particular means of synthesis and includes naturally occurring antibodies isolatable from natural sources and as well as engineered antibodies molecules that are obtained by “recombinant” means including antibodies isolated from transgenic animals that are transgenic for human immunoglobulin genes or a hybridoma prepared therefrom, antibodies isolated from a host cell transformed with a nucleic acid construct that results in expression of an antibody, antibodies isolated from a combinatorial antibody library including phage display libraries. In one embodiment, an “antibody” is a mammalian immunoglobulin is a “full length antibody” comprising variable and constant domains providing binding and effector functions. In most instances, a full-length antibody comprises two light chains and two heavy chains, each light chain comprising a variable region and a constant region. In one embodiment, the antibody is a “full length antibody” comprising two light chains and two heavy chains, each light chain comprising a variable region and a constant region providing binding and effector functions. In a preferred embodiment, the constant and variable regions are “human” (i.e. possessing amino acid sequences characteristic of human immunoglobulins).

CAR or Chimeric Antigen Receptor: As used herein, the terms “chimeric antigen receptor” and “CAR” are used interchangeably to refer to a polyprotein comprising multiple functional domains arranged from amino to carboxy terminus in the sequence: (a) a signal peptide sequence; (b) an extracellular antigen recognition domain (ARD), (c) a transmembrane spanning domain (TSD); (d) one or more intracellular signaling domains (ISDs) wherein the foregoing domains (a)-(d) may optionally be linked by one or more (e) spacer domains. The term “CAR” is also used to refer to a polyprotein as expressed in a cell following post-translational cleavage of the signal peptide sequence, the CAR comprising multiple functional domains arranged from amino to carboxy terminus in the sequence: (a) an extracellular antigen recognition domain (ARD), (b) a transmembrane spanning domain (TSD); (c) one or more intracellular signaling domains (ISDs) wherein the foregoing domains (a)-(d) may optionally be linked by one or more spacer domains.

CAR-T Cell: As used herein, the terms “chimeric antigen receptor T-cell” and “CAR-T cell” are used interchangeably to refer to a T-cell that has been recombinantly modified to express a CAR.

CDR(s): As used herein, the term “CDR” or “complementarity determining region” is intended to mean the non-contiguous antigen combining sites found within the variable region of both heavy and light chain immunoglobulin polypeptides. CDRs have been described by Kabat et al., (1977) J. Biol. Chem. 252:6609-6616; Kabat, et al., U.S. Dept. of Health and Human Services, “Sequences of proteins of immunological interest” (1991) (also referred to herein as Kabat 1991); by Chothia et al. (1987) J. Mol. Biol. 196:901-917; and MacCallum, et al. (1996) J. Mol. Biol. 262:732-745, where the definitions include overlapping or subsets of amino acid residues when compared against each other. Nevertheless, application of either definition to refer to a CDR of an antibody or grafted antibodies or variants thereof is intended to be within the scope of the term as defined and used herein. The numbering of the CDR positions herein is provided according to Kabat numbering conventions.

Circulating Tumor Cell: As used herein the term “circulating tumor cell (CTC)” refers to tumor cells shed from a tumor mass into the peripheral circulation of a subject.

Comparable: As used herein, the term “comparable” is used to describe the degree of difference in two measurements of an evaluable quantitative or qualitative parameter. For example, where a first measurement of an evaluable parameter and a second measurement of the evaluable parameter do not deviate beyond an acceptable range (i.e., a range that the skilled artisan would recognize as not producing a statistically significant difference in effect between the two results in the circumstances) the two measurements would be considered “comparable.” In some instances, measurements may be considered “comparable” if one measurement deviates from another by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one measurement is comparable to a reference standard if it deviates by less than 15%, by less than 10%, or by less than 5% from the reference standard. The term “comparable” may also be used with respect to qualitative as well as quantitative parameters such as improvement non-quantifiable clinically evaluable parameters such as a feeling of well being, appetite, energy, lethargy, and the like.

Derived From: As used herein in the term “derived from” as used in the context of an amino acid sequence or polynucleotide sequence (e.g., an amino acid sequence “derived from” an IL-10 polypeptide), is meant to indicate that the polypeptide or nucleic acid has a sequence that is based on that of a reference polypeptide or nucleic acid (e.g., a naturally occurring IL-10 polypeptide or an IL-10-encoding nucleic acid), and is not meant to be limiting as to the source or method in which the protein or nucleic acid is made. For example, a polypeptide synthesized by solid phase chemical synthesis having a conservative amino acid substitution with respect to a sequence of a naturally occurring polypeptide is considered to be derived from the naturally occurring polypeptide amino acid sequence. By way of example, the term “derived from” includes homologs or variants of reference amino acid or DNA sequences.

Driver Mutation: As used herein the term “driver mutation” refers to a mutation in a neoplastic cell that contributes to the growth and survival of the neoplasm and thereby conferring a selective advantage.

Enriched: As used herein in the term “enriched” refers to a sample is non-naturally manipulated (e.g., by “the hand of man”) so that a molecule of interest is present in: (a) a greater concentration (e.g., at least 3-fold greater, at least 4-fold greater, at least 8-fold greater, at least 64-fold greater, or more) than the concentration of the molecule in a starting sample. The starting sample may be, for example, a sample in which the molecule naturally occurs (e.g. a sample of a naturally occurring material) or in which it is present after administration or that of the environment in which the molecule was synthetically prepared (e.g., sample obtained from a recombinant bacterial cell culture, chemical synthesis, cell culture supernatant, and the like). A sample of a molecule may be have an enhanced level of purity of the molecule with respect to the environment or its synthetic milieu but not substantially pure.

IL-10 Agent: As used herein, the term “IL-10 agent” refers to a dimeric molecule having IL-10 activity comprising two IL-10 polypeptides, the molecule: (a) capable of binding to the IL-10 receptor the binding resulting the modulation of one or more signaling pathways as IL-10 and (b) capable of eliciting a biological response characteristic of IL-10. The term IL-10 agent includes IL-10 molecules which comprise amino acid substitutions, deletions or modifications (IL-10 analogs and IL-10 variants) and modified IL-10 agents (e.g pegylated IL-0).

IL-10 Analog: The term “IL-10 analog” as used herein refers to IL-10 agents that operate through the same mechanism of action as IL-10 (i.e., that bind to and modulate the activity of the IL-10 receptor and agents that modulate the same signaling pathway as IL-10 in a manner analogous thereto) and are capable of eliciting a biological response comparable to (or greater than) that of IL-10.

Polypeptide Analog: The term “polypeptide analog” as used herein refers to polypeptide agents that operate the same mechanism of action of the parent polypeptide from which they are derived (i.e., that specifically bind to and modulate the activity of the parent polypeptide's receptor and agents that modulate the same signaling pathway as parent polypeptide in a manner analogous thereto) and are capable of eliciting a biological response comparable to (or greater than) that of the parent polypeptide. Examples of polypeptide analogs useful in the practice of the present invention include but are not limited to IL-10 polypeptide analogs, IL-12 polypeptide analogs, IL-7 polypeptide analogs, IL-15 polypeptide analogs, IL-2 polypeptide analogs and IL-18 polypeptide analogs

In A Sufficient Amount to Effect a Change: The phrase “in a sufficient amount to effect a change” is used herein to mean that there is a detectable difference between a level of an indicator measured before (e.g., a baseline level) and after administration of a particular agent. Indicators include any objective parameter (e.g., body temperature, serum concentration of IL-10) or subjective parameter (e.g., a subject's feeling of well-being). An amount “sufficient to effect a change” may be a therapeutically effective amount but such amount “sufficient to effect a change” may be more or less than a therapeutically effective amount.

In Combination With: As used herein, the term “in combination with” refers to the administration of a first agent and second agent to a subject. For purposes of the present invention, one agent (e.g. an IL-10 agent) is considered to be administered in combination with a second agent (e.g. a CAR-T cell) if the biological effect resulting from the administration of the first agent persists in the subject at the time of administration of the second agent such that the therapeutic effects of the first agent and second agent overlap. For example, commercially available CAR-T cell therapies (e.g. Kymriah® brand tisagenlecleucel) are typically administered infrequently (or only once) while agents to be combined with such molecule as contemplated by the present disclosure such as hIL-10 or PEGylated hIL-10 are commonly administered daily subcutaneously. However, the administration of the first agent provides a therapeutic effect over an extended time and the administration of the second agent provides its therapeutic effect while the therapeutic effect of the first agent remains ongoing such that the second agent is considered to be administered in combination with the first agent, even though the first agent may have been administered at a point in time significantly distant (e.g. days or weeks) from the time of administration of the second agent. The term “in combination with” also refers to a situation where the first agent and the second agent are administered simultaneously or contemporaneously. In the context of the present disclosure, a first agent is deemed administered simultaneously with a second agent if the first and second agents are administered within 30 minutes of each other. In the context of the present disclosure, a first agent is deemed administered “contemporaneously” with a second agent if first and second agents are administered within about 24 hours minutes of each another, preferably within about 12 hours of each other, preferably within about 6 hours of each other, preferably within about 2 hours of each other, or preferably within about 30 minutes of each other. The term “in combination with” shall also understood to apply to the situation where a first agent and a second agent are co-formulated in single pharmaceutically acceptable formulation and the co-formulation comprising the first and second agents is administered to a subject.

In Need of Treatment: The term “in need of treatment” as used herein refers to a judgment made by a physician or other caregiver with respect to a subject that the subject requires or will potentially benefit from treatment. This judgment is made based on a variety of factors that are in the realm of the physician's or caregiver's expertise.

In Need of Prevention: The term “in need of prevention” as used herein refers to a judgment made by a physician or other caregiver with respect to a subject that the subject requires or will potentially benefit from preventative care. This judgment is made based upon a variety of factors that are in the realm of a physician's or caregiver's expertise.

Inhibitor: Inhibitors are molecules that decrease, block, prevent, delay activation, inactivate, desensitize, or down-regulate, e.g., a gene, protein, ligand, receptor, or cell. An inhibitor can also be defined as a molecule that reduces, blocks, or inactivates a constitutive activity.

Intratumoral Heterogenity: As used herein the term “intratumoral heterogeneity (ITH)” refers to the genetic and phenotypic variation of cells within a tumor in a subject or between individual tumor lesions in the same subject.

Isolated: In the context of a polypeptide, the term “isolated” refers to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

Kabat Numbering: The term “Kabat numbering” as used herein is recognized in the art and refers to a system of numbering amino acid residues which are more variable (e.g., hypervariable) than other amino acid residues in the heavy and light chain regions of immunoglobulins (Kabat, et al., Ann. NY Acad. Sci. 190:382-93 (1971); Kabat, et al., Sequences of Proteins of Immunological Interest, Fifth Edition, U.S. Department of Health and Human Services, NIH Publication No. 91-3242 (1991)). For purposes of the present disclosure, the positioning of CDRs in the variable region of an antibody follows Kabat numbering or simply, “Kabat.”

Ligand: As used herein, the term ligand refers to a molecule that binds to and forms a complex with a biomolecule so as to effect a change in the activity of the biomolecule to which it binds. In one embodiment, the term “ligand” refers to a molecule, or complex thereof, that can act as an agonist or antagonist of a receptor. “Ligand” encompasses natural and synthetic ligands, e.g., cytokines, cytokine variants, analogs, muteins, and binding compositions derived from antibodies. “Ligand” also encompasses small molecules, peptide mimetics of cytokines and peptide mimetics of antibodies. The term ligand also encompasses a molecule that is neither an agonist nor antagonist but that can bind to a receptor while enabling the receptor to retain (or exhibit enhanced) its biological activities (e.g., signaling, catalysis or adhesion). Moreover, the term includes a membrane-bound ligand that has been changed, e.g., by chemical or recombinant methods, to a soluble version of the membrane-bound ligand. A ligand or receptor can be entirely intracellular, that is, it can reside in the cytosol, nucleus, or some other intracellular compartment. The complex of a ligand and receptor is termed a “ligand-receptor complex.”

Metastasis: As used herein the term “metastasis” describes the spread of a cancer cell from a primary tumor to the surrounding tissues and to distant organs of a subject.

Modified Polypeptide Agent: The term “modified polypeptide agents” are polypeptide that have been modified by one or more modifications such as pegylation glycosylation (N- and O-linked); polysialylation; albumin fusion molecules comprising serum albumin (e.g., human serum albumin (HSA), cyno serum albumin, or bovine serum albumin (BSA)); albumin binding through, for example a conjugated fatty acid chain (acylation); and Fc-fusion proteins. Modified IL-10 agents may be prepared to order to enhance one or more properties for example, modulating immunogenicity; methods of increasing water solubility, bioavailability, serum half-life, and/or therapeutic half-life; and/or modulating biological activity. Certain modifications can also be useful to enhance immunogenicity, for example, to raise antibodies for use in detection assays (e.g., immunogenic carrier molecules such as diphtheria or tetantus toxins and fragments and toxoids thereof, epitope tags) and/or facilitate purification (e.g. transition metal ion chelating peptide sequences such as poly-histidyl sequences). Examples of modified polypeptide agents useful in the practice of the present invention include but are not limited to modified polypeptide IL-10 agents, modified polypeptide IL-12 agents, modified polypeptide IL-7 agents, modified polypeptide IL-15 agents, modified polypeptide IL-2 agents and modified polypeptide IL-18 agents.

Modulate: As used herein, the terms “modulate”, “modulation” and the like refer to the ability of an agent to affect a response, either positive or negative or directly or indirectly, in a system, including a biological system or biochemical pathway. The term modulator includes both agonists and antagonists.

Neoplastic Disease: As used herein, the term “neoplastic disease” refers to disorders or conditions in a subject arising from cellular hyper-proliferation or unregulated (or dysregulated) cell replication. The term neoplastic disease refers to disorders arising from the presence of neoplasms in the subject. Neoplasms may be classified as: (1) benign, (2) pre-malignant (or “pre-cancerous”), or (3) malignant (or “cancerous”). The term “neoplastic disease” includes neoplastic-related diseases, disorders and conditions referring to conditions that are associated, directly or indirectly, with neoplastic disease, and includes, e.g., angiogenesis and precancerous conditions such as dysplasia.

N-Terminus: As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

Nucleic Acid: The terms “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

Oncogene Addiction: As used herein the term “oncogene addiction” refers to the phenomenon whereby the survival of a cancer cell depends on the continued activity of a mutated oncogene.

Passenger Mutation: As used herein the term “passenger mutation(s)” refers to a mutation(s) that arise during the development of a neoplasm as a result of increased mutation rates, but do not contribute to growth of the neoplasm.

PD-1: As used herein, the term “PD-1” (or “PD1”) refers to the 288 amino acid polypeptide having the amino acid sequence:

(SEQ ID NO: 58)
MQIPQAPWPV VWAVLQLGWR PGWFLDSPDR PWNPPTFSPA
LLVVTEGDNA TFTCSFSNTS ESFVLNWYRM SPSNQTDKLA
AFPEDRSQPG QDCRFRVTQL PNGRDFHMSV VRARRNDSGT
YLCGAISLAP KAQIKESLRA ELRVTERRAE VPTAHPSPSP
RPAGQFQTLV VGVVGGLLGS LVLLVWVLAV ICSRAARGTI
GARRTGQPLK EDPSAVPVFS VDYGELDFQW REKTPEPPVP
CVPEQTEYAT IVFPSGMGTS SPARRGSADG PRSAQPLRPE
DGHCSWPL

Numbering of amino acid residues in PD-1 refers to the full-length polypeptide shown in SEQ ID NO: 58. Amino acids 1-20 of SEQ ID NO: 58 define a signal sequence that is removed during translational processing resulting in the “mature PD1” molecule comprising amino acids 21-288 of SEQ ID NO 58. Amino acids 171-191 of SEQ ID NO: 58 define the transmembrane domain and resides 192-288 define the cytoplasmic domain. The term PD-1 includes naturally occurring variants including the naturally occurring variant with the substitution of Alanine to Valine at position 215. Amino acids 21-170 define the 150 amino acid extracellular domain of PD-1 having the amino acid sequence:

(SEQ ID NO: 59)
PGWFLDSPDR PWNPPTFSPA LLVVTEGDNA TFTCSFSNTS
ESFVLNWYRM SPSNQTDKLA AFPEDRSQPG QDCRFRVTQL
PNGRDFHMSV VRARRNDSGT YLCGAISLAP KAQIKESLRA
ELRVTERRAE VPTAHPSPSP RPAGQFQTLV

The extracellular domain possesses four glycosylation sites at resides 49, 58, 74 and 116 and a disulfide bond exists between resides 54 and 123.

PD1 Receptor(s): As used herein, the term PD1 receptor refers to either of the group consisting of B7-H1/PD-L1 (hereinafter “PD-L1”) and B7-DC/PD-L2. hereinafter “PD-L2”).

PEG-IL10: Refers to a modified IL-10 agent that has been modified by covalent modification with a polyethylene glycol molecule. The term “PEG-IL-10 agent” refers to a modified IL-10 agent comprising at least one polyethylene glycol (PEG) molecule covalently attached (conjugated) to at least one amino acid residue of an IL-10 polypeptide. The terms “monopegylated IL-10 agent” and “mono-PEG-IL-10 agent” refer to an IL-10 agent with a polyethylene glycol molecule covalently attached to a single amino acid residue on one IL-10 polypeptide of the IL-10 dimer, generally via a linker. As used herein, the terms “dipegylated IL-10” and “di-PEG-IL-10” indicate that at least one polyethylene glycol molecule is attached to a single residue on IL-10 polypeptide of the IL-10 dimer, generally via a linker.

Polypeptide: The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The term polypeptide includes a contiguous polymeric amino acid sequence comprised of multiple functional domains including, but not limited to, fusion proteins with a heterologous amino acid sequence (e.g. chimeric antigen receptors); fusion proteins with heterologous and homologous leader sequences; fusion proteins with or without N-terminus methionine residues; fusion proteins with immunologically tagged proteins; fusion proteins of immunologically active proteins (e.g. antigenic diphtheria or tetanus toxin fragments), and the like.

Prevent: The terms “prevent”, “preventing”, “prevention” and the like refer to a course of action initiated with respect to a subject prior to the onset of a disease, disorder, condition or symptom thereof so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed due to genetic, experiential or environmental factors to having a particular disease, disorder or condition. In certain instances, the terms “prevent”, “preventing”, “prevention” are also used to refer to the slowing of the progression of a disease, disorder or condition to a more harmful or otherwise less desirable state. Prophyactic vaccination is one example of prevention.

Recombinant: As used herein, the term “recombinant” refer to polypeptides and nucleic acids generated using recombinant DNA technology. With respect to a molecule, such as “recombinant human IL-10” or “rhIL-10” is used to denote a molecule produced by recombinant DNA technology such as by host cell transformed with a nucleic acid sequence encoding the molecule (or subunit thereof) so that the molecule is expressed (and optionally secreted from) the transformed host cell. The techniques and protocols for recombinant DNA technology are well known to those of ordinary skill in the art to which this invention pertains.

Response: The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

Small Molecule(s): The term “small molecules” refers to chemical compounds having a molecular weight that is less than about 10 kDa, less than about 2 kDa, or less than about 1 kDa. Small molecules include, but are not limited to, inorganic molecules, organic molecules, organic molecules containing an inorganic component, molecules comprising a radioactive atom, and synthetic molecules. Therapeutically, compared to most large molecules, small molecules have been observed to provide enhanced cell permeability, improved absorption from the gut, reduced immunogenicity, and greater stability particularly at elevated temperature. The term “small molecule” is a term well understood to those of ordinary skill in the pharmaceutical arts.

Specifically Binds: The term “specifically binds” is used herein to refer to the degree of selectivity or affinity for which one molecule binds to another. In the context of binding pairs (e.g. a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, at least ten times greater, at least 20-times greater, or at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the affinity of the antibody for the second molecule of the binding pair is greater than about 10⁹ liters/mole, alternatively greater than about 10¹⁰ liters/mole, greater than about 10¹¹ liters/mole, greater than about 10¹² liters/mole as determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays.

Subject: The terms “patient” or “subject” are used interchangeably to refer to a human or a non-human mammal. Examples of mammalian subjects include but are not limited to members of the superfamilies Cercopithecoidea and Hominoidea, in particular members of the family Hominidae including human beings. The term “subject” also includes members of the families Canidae (including Canisfamiliaris), Felidae (including Felinae and species of the genus Felis, in particular members of specifically including Felis catus), Equidae (specifically including species of the genus Equus such as domesticated horses), and Bovidae (including species of the tribe Bovini such as Bos taurus).

Suffering From: As used herein, the term “suffering from” is used with respect to a disease wherein a determination is made by a physician with respect to a subject based on the available information generally accepted in the field for the identification of a disease, disorder or condition including but not limited to X-ray, CT-scans, conventional laboratory diagnostic tests (e.g. blood count, etc.), genomic data, protein expression data, immunohistochemistry characteristic of a disease state and that the subject requires or will benefit from treatment.

Substantially Pure: As used herein in the term “substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

Therapeutically Effective Amount: The phrase “therapeutically effective amount” as used herein in reference to the administration of an agent to a subject, either alone or as part of a pharmaceutical composition or treatment regimen, in a single dose or as part of a series of doses in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the amount of inflammatory cytokines produced following administration can be indicative of whether a therapeutically effective amount has been used. which contribute to the determination of a therapeutically effective amount of an agent include but are not limited to readily identifiable indicia such as age, weight, sex, general health, ECOG score, observable physiological parameters. Alternatively, or in addition, other parameters commonly assessed in the clinical setting may be monitored to determine if a therapeutically effective amount of an agent has been administered to the subject such as body temperature, heart rate, normalization of blood chemistry, normalization of blood pressure, normalization of cholesterol levels, or any symptom, aspect, or characteristic of the disease, disorder or condition, biomarkers (such as inflammatory cytokines, IFN-γ, granzyme, and the like), reduction in serum tumor markers, improvement in Response Evaluation Criteria In Solid Tumours (RECIST), improvement in Immune-Related Response Criteria (irRC), increase in duration of survival, extended duration of progression free survival, extension of the time to progression, increased time to treatment failure, extended duration of event free survival, extension of time to next treatment, improvement objective response rate, improvement in the duration of response, reduction of tumor burden, complete response, partial response, stable disease, and the like that are relied upon by clinicians in the field for the assessment of an improvement in the condition of the subject in response to administration of an agent. As used herein the terms “Complete Response (CR),” “Partial Response (PR)” “Stable Disease (SD)” and “Progressive Disease (PD)” with respect to target lesions and the terms “Complete Response (CR),” “Incomplete Response/Stable Disease (SD)” and Progressive Disease (PD) with respect to non-target lesions are understood to be as defined in the RECIST criteria. As used herein the terms “immune-related Complete Response (irCR),” “immune-related Partial Response (irPR),” “immune-related Progressive Disease (irPD)” and “immune-related Stable Disease (irSD)” as defined in accordance with the Immune-Related Response Criteria (irRC). As used herein, the term “Immune-Related Response Criteria (irRC)” refers to a system for evaluation of response to immunotherapies as described in Wolchok, et al. (2009) Guidelines for the Evaluation of Immune Therapy Activity in Solid Tumors: Immune-Related Response Criteria, Clinical Cancer Research 15(23): 7412-7420. A therapeutically effective amount may be adjusted over a course of treatment of a subject in connection with the dosing regimen and/or evaluation of the subject's condition and variations in the foregoing factors. In one embodiment, a therapeutically effective amount is an amount of an agent when used alone or in combination with another agent does not result in non-reversible serious adverse events in the course of administration to a mammalian subject.

Treat: The terms “treat”, “treating”, treatment” and the like refer to a course of action (such as administering IL-10, a CAR-T cell, or a pharmaceutical composition comprising same) initiated with respect to a subject after a disease, disorder or condition, or a symptom thereof, has been diagnosed, observed, or the like in the subject so as to eliminate, reduce, suppress, mitigate, or ameliorate, either temporarily or permanently, at least one of the underlying causes of such disease, disorder, or condition afflicting a subject, or at least one of the symptoms associated with such disease, disorder, or condition. The treatment includes a course of action taken with respect to a subject suffering from a disease where the course of action results in the inhibition (e.g., arrests the development of the disease, disorder or condition or ameliorates one or more symptoms associated therewith) of the disease in the subject.

Variant: As used herein, the term “variant” encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Non-naturally-occurring variants include polypeptides and nucleic acids that comprise a change in amino acid or nucleotide sequence, respectively, where the change in sequence is artificially introduced (e.g., muteins); for example, the change is generated in the laboratory by human intervention (“hand of man”). Thus, herein a “mutein” refers broadly to mutated recombinant proteins that usually carry single or multiple amino acid substitutions and are frequently derived from cloned genes that have been subjected to site-directed or random mutagenesis, or from completely synthetic coding sequences. Exemplary IL-10 muteins are described in Eaton, et al. United States Patent Application Publication No. S2015/0038678A1 published Feb. 2, 2015; Hansen, et al. United States Patent Application Publication No. US203/0186386A1 published Oct. 2, 2003 and Van Vlasselaer, et al., United States Patent Application Publication No. US20160068583 A1 published Mar. 10, 2016. Examples of polypeptide analogs useful in the practice of the present invention include but are not limited to IL-10 polypeptide variants, IL-12 polypeptide variants, IL-7 polypeptide variants, IL-15 polypeptide variants, IL-2 polypeptide variants and IL-18 polypeptide variants.

### C. IL-10 Polypeptides

The term “IL-10 polypeptide” is to be broadly construed and include, for example, human and non-human IL-10 related polypeptides, including homologs, variants (including muteins), and fragments thereof, as well as IL-10 polypeptides having, for example, a leader sequence (e.g., the signal peptide), and modified versions of the foregoing. In further particular embodiments, IL-10, IL-10 polypeptide(s), and IL-10 agent(s) are agonists.

The term “IL10 polypeptide” includes IL-10 polypeptides comprising conservative amino acid substitutions. The term “conservative amino acid substitution” refers to substitutions that preserve the activity of the protein by replacing an amino acid(s) in the protein with an amino acid with a side chain of similar acidity, basicity, charge, polarity, or size of the side chain. Conservative amino acid substitutions generally entail substitution of amino acid residues within the following groups: (a) L, I, M, V, F; (b) R, K; (c) F, Y, H, W, R; (d) G, A, T, S; (e) Q, N; and/or (f) D, E. Guidance for substitutions, insertions, or deletions can be based on alignments of amino acid sequences of different variant proteins or proteins from different species. Thus, in addition to any naturally-occurring IL-10 polypeptide, the present disclosure contemplates IL-10 polypeptides having 1, 2, 3, 4, 5, 6, 7, 8, 9, or 10 substitutions, insertions, or deletions. In some embodiments, the IL-10 polypeptide possesses fewer than 20, 10, or 5 amino acid substitutions, insertions, or deletions where the substitution is usually a conservative amino acid substitution.

In some cases, the IL-10 polypeptide includes one or more linkages other than peptide bonds, e.g., at least two adjacent amino acids are joined via a linkage other than an amide bond to reduce or eliminate undesired proteolysis or other means of degradation, and/or to increase serum stability, and/or to restrict or increase conformational flexibility, one or more amide bonds within the backbone of IL-10 can be substituted. One or more amide linkages (—CO—NH—) in an IL-10 polypeptide can be replaced with a linkage which is an isostere of an amide linkage, such as —CH₂NH—, —CH₂S—, —CH₂CH₂—, —CH═CH-(cis and trans), —COCH₂—, —CH(OH)CH₂— or —CH₂SO—. One or more amide linkages in IL-10 can also be replaced by, for example, a reduced isostere pseudopeptide bond. See Couder et al. (1993) Int. J. Peptide Protein Res. 41:181-184. Such replacements and how to affect them are known to those of ordinary skill in the art.

The term “IL10 polypeptide” includes IL-10 polypeptides comprising one or more amino acid substitutions including but not limited to: a) substitution of alkyl-substituted hydrophobic amino acids, including alanine, leucine, isoleucine, valine, norleucine, (S)-2-aminobutyric acid, (S)-cyclohexylalanine or other simple α-amino acids substituted by an aliphatic side chain from C₁-C₁₀ carbons including branched, cyclic and straight chain alkyl, alkenyl or alkynyl substitutions; b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine; c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the α-nitrogen, or the distal nitrogen or nitrogens, or on the α-carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as α-methyl-arginine, α-methyl-2,3-diaminopropionic acid, α-methyl-histidine, α-methyl-ornithine where the alkyl group occupies the pro-R position of the α-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid; d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids; e) substitution of side chain amide residues, including asparagine, glutamine, and alkyl or aromatic substituted derivatives of asparagine or glutamine; and f) substitution of hydroxyl-containing amino acids, including serine, threonine, homoserine, 2,3-diaminopropionic acid, and alkyl or aromatic substituted derivatives of serine or threonine.

The term “IL10 polypeptide” includes IL-10 polypeptides comprising one or more naturally occurring non-genetically encoded L-amino acids, synthetic L-amino acids, or D-enantiomers of an amino acid. For example, IL-10 can comprise only D-amino acids. For example, an IL-10 polypeptide can comprise one or more of the following residues: hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, ω-aminohexanoic acid, ω-aminoheptanoic acid, ω-aminooctanoic acid, ω-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

The term “IL10 polypeptide” includes IL-10 polypeptides comprising one or more additional cysteine residues or cysteine analogs to facilitate linkage of the IL-10 polypeptide to another polypeptide via a disulfide linkage or to provide for cyclization of the IL-10 polypeptide. Methods of introducing a cysteine or cysteine analog are known in the art; see, e.g., U.S. Pat. No. 8,067,532.

The term “IL10 polypeptide” includes cyclized polypeptides. A cyclizing bond can be generated with any combination of amino acids (or with an amino acid and —(CH₂)ₙ—CO— or —(CH₂)ₙ—C₆H₄—CO—) with functional groups which allow for the introduction of a bridge. Some examples are disulfides, disulfide mimetics such as the —(CH₂)ₙ— carba bridge, thioacetal, thioether bridges (cystathionine or lanthionine) and bridges containing esters and ethers. In these examples, n can be any integer, but is frequently less than ten.

The term “IL10 polypeptide” includes additional modifications including, for example, an N-alkyl (or aryl) substitution (W[CONR]), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

The term “IL10 polypeptide” includes a retroinverso analog (see, e.g., Sela and Zisman (1997) FASEB J. 11:449). Retro-inverso peptide analogs are isomers of linear polypeptides in which the direction of the amino acid sequence is reversed (retro) and the chirality, D- or L-, of one or more amino acids therein is inverted (inverso), e.g., using D-amino acids rather than L-amino acids. [See, e.g., Jameson et al. (1994) Nature 368:744; and Brady et al. (1994) Nature 368:692].

The term “IL10 polypeptide” includes modifications to include a “Protein Transduction Domain” (PTD). The term “protein transcution domain” refers to a polypeptide, polynucleotide, carbohydrate, or organic or inorganic molecule that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an IL-10 polypeptide, while in other embodiments, a PTD is covalently linked to the carboxyl terminus of an IL-10 polypeptide. Exemplary protein transduction domains include, but are not limited to, a minimal undecapeptide protein transduction domain (corresponding to residues 47-57 of HIV-1 TAT comprising YGRKKRRQRRR; SEQ ID NO: 1); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO: 2); Transportan GWTLNSAGYLLGKINLKALAALAKKIL (SEQ ID NO: 3); KALAWEAKLAKALAKALAKHLAKALAKALKCEA (SEQ ID NO: 4); and RQIKIWFQNRRMKWKK (SEQ ID NO: 5). Exemplary PTDs include, but are not limited to, YGRKKRRQRRR (SEQ ID NO: 6), RKKRRQRRR (SEQ ID NO: 7); an arginine homopolymer of from 3 arginine residues to 50 arginine residues; exemplary PTD domain amino acid sequences include, but are not limited to, any of the following: YGRKKRRQRRR (SEQ ID NO: 8); RKKRRQRR (SEQ ID NO: 9); YARAAARQARA (SEQ ID NO: 10); THRLPRRRRRR (SEQ ID NO: 11); and GGRRARRRRRR (SEQ ID NO: 12).

The carboxyl group COR₃ of the amino acid at the C-terminal end of an IL-10 polypeptide can be present in a free form (R3=OH) or in the form of a physiologically-tolerated alkaline or alkaline earth salt such as, e.g., a sodium, potassium or calcium salt. The carboxyl group can also be esterified with primary, secondary or tertiary alcohols such as, e.g., methanol, branched or unbranched C₁-C₆-alkyl alcohols, e.g., ethyl alcohol or tert-butanol. The carboxyl group can also be amidated with primary or secondary amines such as ammonia, branched or unbranched C₁-C₆-alkylamines or C₁-C₆ di-alkylamines, e.g., methylamine or dimethylamine.

The amino group of the amino acid NR1R2 at the N-terminus of an IL-10 polypeptide can be present in a free form (R1=H and R2=H) or in the form of a physiologically-tolerated salt such as, e.g., a chloride or acetate. The amino group can also be acetylated with acids such that R1=H and R2=acetyl, trifluoroacetyl, or adamantyl. The amino group can be present in a form protected by amino-protecting groups conventionally used in peptide chemistry, such as those provided above (e.g., Fmoc, Benzyloxy-carbonyl (Z), Boc, and Alloc). The amino group can be N-alkylated in which R₁ and/or R₂=C₁-C₆ alkyl or C₂-C₈ alkenyl or C₇-C₉ aralkyl. Alkyl residues can be straight-chained, branched or cyclic (e.g., ethyl, isopropyl and cyclohexyl, respectively).

The term “IL10 polypeptide” includes active fragments of IL-10 polypeptides. The term “active IL-10 polypeptide fragment” refers to IL-10 polypeptides that are fragments (e.g., subsequences) of naturally occurring IL-10 species containing contiguous amino acid residues derived from the naturally occurring IL-10 species are capable of dimerizing with another IL-10 polypeptide such dimer possessing IL-10 activity. The length of contiguous amino acid residues of a peptide or a polypeptide subsequence varies depending on the specific naturally-occurring amino acid sequence from which the subsequence is derived. In general, peptides and polypeptides can be from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide. The term “active fragments of IL-10 polypeptides” includes IL-10 polypeptides comprising deletions of 1, 2, 3, 4, 5, 6, 7, 8, 9, 10, 11, 12, 13, 14, 15, 16, 17, 18, 19, 20, 21, 22, or 23 amino acids from the N-terminus of the mature (i.e. not including the signal peptide sequence) IL-10 polypeptide. The term “active fragments of IL-10 polypeptides” includes IL-10 polypeptides comprising deletions of 1, 2, 3, 4, 5, 6, 7, 8, 9, 10, 11, 12 or 13 amino acids from the C-terminus of the mature (i.e. not including the signal peptide sequence) IL-10 polypeptide.

Additionally, IL-10 polypeptides can have a defined sequence identity compared to a reference sequence over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, (1981) Adv. Appl. Math. 2:482, by the homology alignment algorithm of Needleman & Wunsch (1970) J. Mol. Biol. 48:443, by the search for similarity method of Pearson & Lipman (1988) Proc. Nat'l. Acad. Sci. USA 85:2444, by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)). Software packages and databases for determining, e.g., antigenic fragments, leader sequences, protein folding, functional domains, glycosylation sites, and sequence alignments, are available (see, e.g., GCG Wisconsin Package (Accelrys, Inc., San Diego, CA); and DeCypher™ (TimeLogic Corp., Crystal Bay, NV).

As an example, a suitable IL-10 polypeptide can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide.

As discussed further below, the IL-10 polypeptides can be isolated from a natural source (e.g., an environment other than its naturally-occurring environment) and can also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-10 polypeptides can also be synthetically produced (e.g., by cell-free chemical synthesis).

The present disclosure contemplates IL-10 agents comprised of IL-10 polypeptides obtained from a variety of mammalian and non-mammalian sources including orthologs, and modified forms thereof. In addition to the human polypeptides and the nucleic acid molecules which encode them, the present disclosure contemplates IL-10 polypeptides and corresponding nucleic acid molecules from other species including murine, rat (accession NP_036986.2; GI 148747382); cow (accession NP_776513.1; GI 41386772); sheep (accession NP_001009327.1; GI 57164347); dog (accession ABY86619.1; GI 166244598); and rabbit (accession AAC23839.1; GI 3242896). Examples of IL-10 agents derived from non-mammalian sources include viral IL-10 derived from the family herpesviridae subfamily betaherpesvirinae, genus cytomegalovirus including human cytomegalovirus, Genbank Accession Nos. AAR31656 and ACR49217), green monkey cytomegalovirus, (Genbank Accession No AEV80459), rhesus cytomegalovirus, (Genbank Accession No. AAF59907), baboon cytomegalovirus, (Genbank Accession No. AAF63436), owl monkey cytomegalovirus, (Genbank Accession No. AEV80800), and squirrel monkey cytomegalovirus, (Genbank Accession No. AEV80955; family Gammaherpesvirinae genus lymphocryptovirus Epstein-Barr virus, (Genbank Accession No. CAD53385), bonobo herpesvirus, (Genbank Accession No. XP_003804206.1), Rhesus lymphocryptovirus, (Genbank Accession No. AAK95412), baboon lymphocryptovirus, (Genbank Accession No. AAF23949); genus macavirus including ovine herpesvirus 2 (Genbank Accession No. AAX58040); genus percavirus including equid herpesvirus 2 (Genbank Accession No. AAC13857); family alloherpesviridea genus cyprinivirus including cyprinid herpesvirus 3 (Genbank Accession No. ABG429610), anguillid herpesvirus 1 (Genbank Accession No. AFK25321); family poxviridae, subfamily chodopoxvirinae genus parapoxvirus including orf virus (Genbank Accession No. AAR98352), bovine papular stomatitis virus (Genbank Accession No AAR98483), pseudocowpox virus (Genbank Accession No. ADC53770); genus capripoxvirus including lumpy skin disease virus (Genbank Accession No AAK84966), sheeppox virus (Genbank Accession No. NP_659579), goatpox virus (Genbank Accession No. YP_00129319 and avipoxvirus including canarypox virus (Genbank Accession No NP_955041).

In one embodiment, the IL-10 polypeptide is a human IL-10 polypeptide. As used herein, the term “human IL-10” or “hIL10” refers to an IL10 agent comprised of two human iIL-10 polypeptides. In one embodiment, a human IL-10 polypeptide is a 160 amino acid polypeptide having the amino acid sequence (amino- to carboxy-terminus):

(SEQ ID NO. 13)
SPGQGTQSEN SCTHFPGNLP NMLRDLRDAF SRVKTFFQMK
DQLDNLLLKE SLLEDFKGYL GCQALSEMIQ FYLEEVMPQA
ENQDPDIKAH VNSLGENLKT LRLRLRRCHR FLPCENKSKA
VEQVKNAFNK LQEKGIYKAM SEFDIFINYI EAYMTMKIRN

In one embodiment, a human IL-10 polypeptide is a 161 amino acid polypeptide having the amino acid sequence (amino- to carboxy-terminus):

(SEQ ID NO. 14)
MSPGQGTQSE NSCTHFPGNL PNMLRDLRDA FSRVKTFFQM
KDQLDNLLLK ESLLEDFKGY LGCQALSEMI QFYLEEVMPQ
AENQDPDIKA HVNSLGENLK TLRLRLRRCH RFLPCENKSK
AVEQVKNAFN KLQEKGIYKA MSEFDIFINY IEAYMTMKIR N

In one embodiment, a human IL-10 polypeptide is a 161 amino acid polypeptide having the amino acid sequence (amino- to carboxy-terminus):

(SEQ ID NO. 15)
N-formyl-MSPGQGTQSE NSCTHFPGNL PNMLRDLRDA
FSRVKTFFQM KDQLDNLLLK ESLLEDFKGY LGCQALSEMI
QFYLEEVMPQ AENQDPDIKA HVNSLGENLK TLRLRLRRCH
RFLPCENKSK AVEQVKNAFN KLQEKGIYKA MSEFDIFINY
IEAYMTMKIR N

It should be noted that any reference to “human” in connection with the polypeptides and nucleic acid molecules of the present disclosure is not meant to be limiting with respect to the manner in which the polypeptide or nucleic acid is obtained or the source, but rather is only with reference to the sequence as it can correspond to a sequence of a naturally occurring human polypeptide or nucleic acid molecule.

### D. IL-10 Activity

The term “IL-10 activity” is refers to IL-10 agents typically exert their effects by binding to the IL-10 receptor. The IL-10 receptor, a type II cytokine receptor, consists of alpha and beta subunits, which are also referred to as R1 and R2, respectively. Receptor activation requires binding to both alpha and beta. One IL-10 monomer of the dimeric IL-10 binds to alpha and the other IL-10 monomer of the IL-10 binds to beta. IL-10 activity may be assessed by assays well known in the art. For example, the IL-10 activity of an IL-10 agent may be determined in using the TNF-α inhibition assay, MC9 proliferation assay, CD8 T-cell IFNγ Secretion Assay or in tumor models and tumor analysis as provided below. However, it is understood by the skilled artisan that the following assays are representative, and not exclusionary of, assays to determine IL-10 activity. The skilled artisan will understand that any art recognized assay or methodology to measure IL-10 activity may be used alone or in combination to evaluate the activity of the IL-10 agents described herein.

The IL-10 activity of an IL-10 agent may be assessed in substantial accordance with the following TNFα inhibition assay. Briefly, PMA-stimulation of U937 cells (lymphoblast human cell line from lung available from Sigma-Aldrich (#85011440); St. Louis, MO) causes the cells to secrete TNFα, and subsequent treatment of these TNFα-secreting cells with a test agent having IL-10 activity will result in a decrease in TNFα secretion in a dose-dependent manner. An exemplary TNFα inhibition assay can be performed using the following protocol. After culturing U937 cells in RMPI containing 10% FBS/FCS and antibiotics, plate 1×105, 90% viable U937 cells in 96-well flat bottom plates (any plasma-treated tissue culture plates (e.g., Nunc; Thermo Scientific, USA) can be used) in triplicate per condition. Plate cells to provide for the following conditions (all in at least triplicate; for ‘media alone’ the number of wells is doubled because one-half will be used for viability after incubation with 10 nM PMA): 5 ng/ml LPS alone; 5 ng/mL LPS+0.1 ng/mL rhIL-10; 5 ng/mL LPS+1 ng/mL rhIL-10; 5 ng/mL LPS+10 ng/mL rhIL-10; 5 ng/mL LPS+100 ng/mL rhIL-10; 5 ng/mL LPS+1000 ng/mL rhIL-10; 5 ng/mL LPS+0.1 ng/mL PEG-rhIL-10; 5 ng/mL LPS+1 ng/mL PEG-rhIL-10; 5 ng/mL LPS+10 ng/mL PEG-rhIL-10; 5 ng/mL LPS+100 ng/mL PEG-rhIL-10; and 5 ng/mL LPS+1000 ng/mL PEG-rhIL-10. Expose each well to 10 nM PMA in 200 μL for 24 hours, culturing at 37 °C in 5% CO₂ incubator, after which time ~90% of cells should be adherent. The three extra wells are re-suspended, and the cells are counted to assess viability (>90% should be viable). Wash gently but thoroughly 3× with fresh, non-PMA—containing media, ensuring that cells are still in the wells. Add 100 μL per well of media containing the appropriate concentrations (2× as the volume will be diluted by 100%) of the IL-10 agent, incubate at 37 °C in a 5% CO₂ incubator for 30 minutes. Add 100 μL per well of 10 ng/mL stock LPS to achieve a final concentration of 5 ng/mL LPS in each well and incubate at 37 °C in a 5% CO₂ incubator for 18-24 hours. Remove supernatant and perform TNFα ELISA according to the manufacturer's instructions. Run each conditioned supernatant in duplicate in ELISA.

The IL-10 activity of an IL-10 agent may be assessed in substantial accordance with the following MC/9 cell proliferation assay. Briefly, the administration of compounds having IL-10 activity to MC/9 cells causes increased cell proliferation in a dose-dependent manner. MC/9 is a murine cell line with characteristics of mast cells available from Cell Signaling Technology; Danvers, MA. Thompson-Snipes, L. et al. ((1991) J. Exp. Med. 173:507-10) describe a standard assay protocol in which MC/9 cells are supplemented with IL3+IL10 and IL3+IL4+IL10. Those of ordinary skill in the art will be able to modify the standard assay protocol described in Thompson-Snipes, L. et al, such that cells are only supplemented with IL-10.

The IL-10 activity of an IL-10 agent may be assessed in substantial accordance with the following CD8 T-cell IFNγ Secretion Assay. Briefly, activated primary human CD8 T-cells secrete IFNγ when treated with compounds having IL-10 activity and then with an anti-CD3 antibody. The following protocol provides an exemplary CD8 T-cell IFNγ secretion assay. Human primary peripheral blood mononuclear cells (PBMCs) can be isolated according to any standard protocol (see, e.g., Fuss et al. (2009) Current Protocols in Immunology, Unit 7.1, John Wiley, Inc., NY). 2.5 mL of PBMCs (at a cell density of 10 million cells/mL) can be cultured per well with complete RPMI, containing RPMI (Life Technologies; Carlsbad, CA), 10 mM HEPES (Life Technologies; Carlsbad, CA), 10% Fetal Calf Serum (Hyclone Thermo Fisher Scientific; Waltham, MA) and Penicillin/Streptomycin cocktail (Life Technologies; Carlsbad, CA), in any standard tissue culture treated 6-well plate (BD; Franklin Lakes, NJ). The IL-10 agent is then added to the wells at a final concentration of 100 ng/mL; a final concentration of 10 μg/mL of antibodies blocking the function of inhibitory/checkpoint receptors can also be added in combination with the IL-10 agent. Cells can be incubated in a humidified 37 °C incubator with 5% CO₂ for 6-7 days. After incubation, CD8 T-cells are isolated using Miltenyi Biotec's MACS cell separation technology in substantial accordance with the manufacturer's instructions (Miltenyi Biotec; Auburn, CA). The isolated CD8 T-cells can then be cultured with complete RPMI containing 1 μg/mL anti-CD3 antibody (Affymetrix eBioscience; San Diego, CA) in any standard tissue culture plate for 4 hours. After the 4 hour incubation, the media is collected and assayed for IFNγ using a commercial ELISA kit (e.g. Affymetrix eBioscience; San Diego, CA) in substantial accordance with the manufacturer's instructions.

Tumor models can be used to evaluate the activity of an IL-10 agent on various tumors. The tumor models and tumor analyses described hereafter are representative of those that can be utilized. Syngeneic mouse tumor cells are injected subcutaneously or intradermally at 10⁴, 10⁵ or 10⁶ cells per tumor inoculation. Ep2 mammary carcinoma, CT26 colon carcinoma, PDV6 squamous carcinoma of the skin and 4T1 breast carcinoma models can be used (see, e.g., Langowski et al. (2006) Nature 442:461-465). Immunocompetent Balb/C or B-cell deficient Balb/C mice can be used. IL-10 agents based on murine IL-10 species may be administered to immunocompetent mice, IL-10 agents based on human IL-10 or other non-murine species treatment is typically provided in the B-cell deficient mice. Tumor growth is typically monitored twice weekly using electronic calipers. Tumor volume can be calculated using the formula (width²×length/2) where length is the longer dimension. Tumors are allowed to reach a size of 90-250 mm³ before administration of the IL-10 test agent. The IL-10 agent or buffer control is administered at a site distant from the tumor implantation. Tumor growth following administration of the IL-10 test agent is typically monitored twice weekly using electronic calipers as above and the effects on tumor volume in response to the administration of the IL-10 test agent evaluated over time. Tumor tissues and lymphatic organs are harvested at various endpoints to measure mRNA expression for a number of inflammatory markers and to perform immunohistochemistry for several inflammatory cell markers. The tissues are snap-frozen in liquid nitrogen and stored at −80 °C.

### E. Obtaining IL-10 Polypeptides

IL-10 polypeptides can be isolated from a natural source (e.g., an environment other than its naturally-occurring environment) and can also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-10 polypeptides can also be synthetically produced (e.g., by cell-free or solid phase chemical synthesis).

Where an IL-10 polypeptide is chemically synthesized, the synthesis can proceed via liquid-phase or solid-phase. Solid-phase peptide synthesis (SPPS) allows the incorporation of unnatural amino acids and/or peptide/protein backbone modification. Various forms of SPPS, such as 9-fluorenylmethoxycarbonyl (Fmoc) and t-butyloxycarbonyl (Boc), are available for synthesizing polypeptides of the present disclosure. Details of the chemical syntheses are known in the art (e.g., Ganesan A. (2006) Mini Rev. Med. Chem. 6:3-10; and Camarero J. A. et al., (2005) Protein Pept Lett. 12:723-8).

Solid phase peptide synthesis can be performed as described hereafter. The alpha functions (Nα) and any reactive side chains are protected with acid-labile or base-labile groups. The protective groups are stable under the conditions for linking amide bonds but can readily be cleaved without impairing the peptide chain that has formed. Suitable protective groups for the α-amino function include, but are not limited to, the following: Boc, benzyloxycarbonyl (Z), O-chlorbenzyloxycarbonyl, bi-phenylisopropyloxycarbonyl, tert-amyloxycarbonyl (Amoc), α, α-dimethyl-3,5-dimethoxy-benzyloxycarbonyl, o-nitrosulfenyl, 2-cyano-t-butoxy-carbonyl, Fmoc, 1-(4,4-dimethyl-2,6-dioxocylohex-1-ylidene)ethyl (Dde) and the like.

Suitable side chain protective groups include, but are not limited to: acetyl, allyl (All), allyloxycarbonyl (Alloc), benzyl (Bzl), benzyloxycarbonyl (Z), t-butyloxycarbonyl (Boc), benzyloxymethyl (Bom), o-bromobenzyloxycarbonyl, t-butyl (tBu), t-butyldimethylsilyl, 2-chlorobenzyl, 2-chlorobenzyloxycarbonyl, 2,6-dichlorobenzyl, cyclohexyl, cyclopentyl, 1-(4,4-dimethyl-2,6-dioxocyclohex-1-ylidene)ethyl (Dde), isopropyl, 4-methoxy-2,3-6-trimethylbenzylsulfonyl (Mtr), 2,3,5,7,8-pentamethylchroman-6-sulfonyl (Pmc), pivalyl, tetrahydropyran-2-yl, tosyl (Tos), 2,4,6-trimethoxybenzyl, trimethylsilyl and trityl (Trt).

In the solid phase synthesis, the C-terminal amino acid is coupled to a suitable support material. Suitable support materials are those which are inert towards the reagents and reaction conditions for the step-wise condensation and cleavage reactions of the synthesis process and which do not dissolve in the reaction media being used. Examples of commercially-available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. When preparation of the peptidic acid is desired, polystyrene (1%)-divinylbenzene or TentaGel® derivatized with 4-benzyloxybenzyl-alcohol (Wang-anchor) or 2-chlorotrityl chloride can be used. In the case of the peptide amide, polystyrene (1%) divinylbenzene or TentaGel® derivatized with 5-(4′-aminomethyl)-3′,5′-dimethoxyphenoxy)valeric acid (PAL-anchor) or p-(2,4-dimethoxyphenyl-amino methyl)-phenoxy group (Rink amide anchor) can be used.

The linkage to the polymeric support can be achieved by reacting the C-terminal Fmoc-protected amino acid with the support material by the addition of an activation reagent in ethanol, acetonitrile, N,N-dimethylformamide (DMF), dichloromethane, tetrahydrofuran, N-methylpyrrolidone or similar solvents at room temperature or elevated temperatures (e.g., between 40 °C and 60 °C) and with reaction times of, e.g., 2 to 72 hours.

The coupling of the Nα-protected amino acid (e.g., the Fmoc amino acid) to the PAL, Wang or Rink anchor can, for example, be carried out with the aid of coupling reagents such as N,N′-dicyclohexylcarbodiimide (DCC), N,N′-diisopropylcarbodiimide (DIC) or other carbodiimides, 2-(1H-benzotriazol-1-yl)-1,1,3,3-tetramethyluronium tetrafluoroborate (TBTU) or other uronium salts, O-acyl-ureas, benzotriazol-1-yl-tris-pyrrolidino-phosphonium hexafluorophosphate (PyBOP) or other phosphonium salts, N-hydroxysuccinimides, other N-hydroxyimides or oximes in the presence or absence of 1-hydroxybenzotriazole or 1-hydroxy-7-azabenzotriazole, e.g., with the aid of TBTU with addition of HOBt, with or without the addition of a base such as, for example, diisopropylethylamine (DIEA), triethylamine or N-methylmorpholine, e.g., diisopropylethylamine with reaction times of 2 to 72 hours (e.g., 3 hours in a 1.5 to 3-fold excess of the amino acid and the coupling reagents, for example, in a 2-fold excess and at temperatures between about 10 °C and 50 °C, for example, 25 °C in a solvent such as dimethylformamide, N-methylpyrrolidone or dichloromethane, e.g., dimethylformamide).

Instead of the coupling reagents, it is also possible to use the active esters (e.g., pentafluorophenyl, p-nitrophenyl or the like), the symmetric anhydride of the Nα-Fmoc-amino acid, its acid chloride or acid fluoride, under the conditions described above.

The Nα-protected amino acid (e.g., the Fmoc amino acid) can be coupled to the 2-chlorotrityl resin in dichloromethane with the addition of DIEA and having reaction times of 10 to 120 minutes, e.g., 20 minutes, but is not limited to the use of this solvent and this base.

The successive coupling of the protected amino acids can be carried out according to conventional methods in peptide synthesis, typically in an automated peptide synthesizer. After cleavage of the Nα-Fmoc protective group of the coupled amino acid on the solid phase by treatment with, e.g., piperidine (10% to 50%) in dimethylformamide for 5 to 20 minutes, e.g., 2×2 minutes with 50% piperidine in DMF and 1×15 minutes with 20% piperidine in DMF, the next protected amino acid in a 3 to 10-fold excess, e.g., in a 10-fold excess, is coupled to the previous amino acid in an inert, non-aqueous, polar solvent such as dichloromethane, DMF or mixtures of the two and at temperatures between about 10 °C and 50 °C, e.g., at 25 °C. The previously mentioned reagents for coupling the first Nα-Fmoc amino acid to the PAL, Wang or Rink anchor are suitable as coupling reagents. Active esters of the protected amino acid, or chlorides or fluorides or symmetric anhydrides thereof can also be used as an alternative.

At the end of the solid phase synthesis, the peptide is cleaved from the support material while simultaneously cleaving the side chain protecting groups. Cleavage can be carried out with trifluoroacetic acid or other strongly acidic media with addition of 5%-20% V/V of scavengers such as dimethylsulfide, ethylmethylsulfide, thioanisole, thiocresol, m-cresol, anisole ethanedithiol, phenol or water, e.g., 15% v/v dimethylsulfide/ethanedithiol/m-cresol 1:1:1, within 0.5 to 3 hours, e.g., 2 hours. Peptides with fully protected side chains are obtained by cleaving the 2-chlorotrityl anchor with glacial acetic acid/trifluoroethanol/dichloromethane 2:2:6. The protected peptide can be purified by chromatography on silica gel. If the peptide is linked to the solid phase via the Wang anchor and if it is intended to obtain a peptide with a C-terminal alkylamidation, the cleavage can be carried out by aminolysis with an alkylamine or fluoroalkylamine. The aminolysis is carried out at temperatures between about −10 °C and 50 °C (e.g., about 25 °C), and reaction times between about 12 and 24 hours (e.g., about 18 hours). In addition, the peptide can be cleaved from the support by re-esterification, e.g., with methanol.

The acidic solution that is obtained can be admixed with a 3 to 20-fold amount of cold ether or n-hexane, e.g., a 10-fold excess of diethyl ether, in order to precipitate the peptide and hence to separate the scavengers and cleaved protective groups that remain in the ether. A further purification can be carried out by re-precipitating the peptide several times from glacial acetic acid. The precipitate that is obtained can be taken up in water or tert-butanol or mixtures of the two solvents, e.g., a 1:1 mixture of tert-butanol/water, and freeze-dried.

The peptide obtained can be purified by various chromatographic methods, including ion exchange over a weakly basic resin in the acetate form; hydrophobic adsorption chromatography on non-derivatized polystyrene/divinylbenzene copolymers (e.g., Amberlite® XAD); adsorption chromatography on silica gel; ion exchange chromatography, e.g., on carboxymethyl cellulose; distribution chromatography, e.g., on Sephadex® G-25; countercurrent distribution chromatography; or high pressure liquid chromatography (HPLC) e.g., reversed-phase HPLC on octyl or octadecylsilylsilica (ODS) phases.

Methods describing the preparation of human and mouse IL-10 can be found in, for example, U.S. Pat. No. 5,231,012, which teaches methods for the production of proteins having IL-10 activity, including recombinant and other synthetic techniques. IL-10 can be of viral origin, and the cloning and expression of a viral IL-10 from Epstein Barr virus (BCRF1 protein) is disclosed in Moore, et al., (1990) Science 248:1230. IL-10 can be obtained in a number of ways using standard techniques known in the art, such as those described herein. Recombinant human IL-10 is also commercially available, e.g., from PeproTech, Inc., Rocky Hill, N.J.

Nucleic acid molecules encoding the IL-10 agents are contemplated by the present disclosure, including their naturally-occurring and non-naturally occurring isoforms, allelic variants and splice variants. The present disclosure also encompasses nucleic acid sequences that vary in one or more bases from a naturally-occurring DNA sequence but still translate into an amino acid sequence that corresponds to an IL-10 polypeptide due to degeneracy of the genetic code.

Where a polypeptide is produced using recombinant techniques, the polypeptide can be produced as an intracellular protein or as a secreted protein, using any suitable construct and any suitable host cell, which can be a prokaryotic or eukaryotic cell, such as a bacterial (e.g., E. coli) or a yeast host cell, respectively. Other examples of eukaryotic cells that can be used as host cells include insect cells, mammalian cells, and/or plant cells. Where mammalian host cells are used, they can include human cells (e.g., HeLa, 293, H9 and Jurkat cells); mouse cells (e.g., NIH3T3, L cells, and C127 cells); primate cells (e.g., Cos 1, Cos 7 and CV1); and hamster cells (e.g., Chinese hamster ovary (CHO) cells).

A variety of host-vector systems suitable for the expression of a polypeptide can be employed according to standard procedures known in the art. See, e.g., Sambrook, et al., (1989) Current Protocols in Molecular Biology Cold Spring Harbor Press, New York; and Ausubel, et al. (1995) Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences and can provide for inducible or constitutive expression where the coding region is operably-linked under the transcriptional control of the transcriptional initiation region, and a transcriptional and translational termination region. In general, the transcriptional and translational regulatory sequences can include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7).

Expression constructs generally have convenient restriction sites located near the promoter sequence to provide for the insertion of nucleic acid sequences encoding proteins of interest. A selectable marker operative in the expression host can be present to facilitate selection of cells containing the vector. Moreover, the expression construct can include additional elements. For example, the expression vector can have one or two replication systems, thus allowing it to be maintained in organisms, for example, in mammalian or insect cells for expression and in a prokaryotic host for cloning and amplification. In addition, the expression construct can contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture by immunoaffinity purification, which generally involves contacting the sample with an anti-protein antibody, washing to remove non-specifically bound material, and eluting the specifically bound protein. The isolated protein can be further purified by dialysis and other methods normally employed in protein purification. In one embodiment, the protein can be isolated using metal chelate chromatography methods. Proteins can contain modifications to facilitate isolation.

The polypeptides can be prepared in substantially pure or isolated form (e.g., free from other polypeptides). The polypeptides can be present in a composition that is enriched for the polypeptide relative to other components that can be present (e.g., other polypeptides or other host cell components). For example, purified polypeptide can be provided such that the polypeptide is present in a composition that is substantially free of other expressed proteins, e.g., less than about 90%, less than about 60%, less than about 50%, less than about 40%, less than about 30%, less than about 20%, less than about 10%, less than about 5%, or less than about 1%.

An IL-10 polypeptide can be generated using recombinant techniques to manipulate different IL-10—related nucleic acids known in the art to provide constructs capable of encoding the IL-10 polypeptide. It will be appreciated that, when provided a particular amino acid sequence, the ordinary skilled artisan will recognize a variety of different nucleic acid molecules encoding such amino acid sequence in view of her background and experience in, for example, molecular biology.

### F. PEGylated IL-10

In one embodiment, the modified IL-10 agent is a PEG-IL10 agent. Pegylation of IL-10 agents results in improvement of certain properties including pharmacokinetic parameters (e.g., serum half-life), enhancement of activity, improved physical and thermal stability, protection against susceptibility to enzymatic degradation, increased solubility, longer in vivo circulating half-life and decreased clearance, reduced immunogenicity and antigenicity, and reduced toxicity. In addition to the beneficial effects of pegylation on pharmacokinetic parameters, pegylation itself can enhance activity. For example, PEG-IL-10 has been shown to be more efficacious against certain cancers than unpegylated IL-10 (see, e.g., EP 206636A2).

In certain embodiments, the PEG-IL-10 agent used in the present disclosure is a mono-PEG-IL-10 agent in which one to nine PEG molecules are covalently attached via a linker to the α-amino group of the amino acid residue at the N-terminus of one IL-10 polypeptide of the IL-10 dimer. Monopegylation of one IL-10 polypeptide generally results in a non-homogeneous mixture of non-pegylated, monopegylated and dipegylated IL-10 polypeptides due to subunit shuffling. Particular embodiments of the present disclosure comprise the administration of a mixture of mono- and di-pegylated IL-10 agents produced by the methods described herein. In particular embodiments, the mixture of mono and di-pegylated IL-10 is an approximately 1:1 ratio of mono and di-pegylated rhIL-10 prepared in substantial accordance with the teaching of Blaisdell, et al. U.S. Pat. No. 8,691,205B2 issued Apr. 8, 2014, the entire teaching of which is herein incorporated by reference, and Blaisdell, European patent No 2379115B1 (granted Oct. 25, 2017).

The biological activity PEG-IL-10 agents may by assessed by the levels of inflammatory cytokines (e.g., TNF-α or IFN-γ) in the serum of subjects challenged with a bacterial antigen (lipopolysaccharide (LPS)) and treated with PEG-IL-10, as described in U.S. Pat. No. 7,052,686.

Although the method or site of PEG attachment to IL-10 is not critical, in certain embodiments the pegylation does not alter, or only minimally alters, the activity of the IL-10 agent. In certain embodiments, the increase in half-life is greater than any decrease in biological activity.

PEGs suitable for conjugation to a IL-10 polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched.

Branched PEG derivatives, “star-PEGs” and multi-armed PEGs are contemplated by the present disclosure.

A molecular weight of the PEG used in the present disclosure is not restricted to any particular range. The PEG component of the PEG-IL-10 agent can have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods known in the art. Chromatography may be used to resolve conjugate fractions, and a fraction is then identified which contains the conjugate having, for example, the desired number of PEGs attached, purified free from unmodified protein sequences and from conjugates having other numbers of PEGs attached.

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons.

Two widely used first generation activated monomethoxy PEGs (mPEGs) are succinimdyl carbonate PEG (SC-PEG; see, e.g., Zalipsky, et al. (1992) Biotehnol. Appl. Biochem 15:100-114; and Miron and Wilcheck (1993) Bio-conjug. Chem. 4:568-569) and benzotriazole carbonate PEG (BTC-PEG; see, e.g., Dolence, et al. U.S. Pat. No. 5,650,234), which react preferentially with lysine residues to form a carbamate linkage but are also known to react with histidine and tyrosine residues. The linkage to histidine residues on certain molecules (e.g., IFNα) has been shown to be a hydrolytically unstable imidazolecarbamate linkage (see, e.g., Lee and McNemar, U.S. Pat. No. 5,985,263). Second generation pegylation technology has been designed to avoid these unstable linkages as well as the lack of selectivity in residue reactivity. Use of a PEG-aldehyde linker targets a single site on the N-terminus of a polypeptide through reductive amination.

The PEG conjugated to the polypeptide sequence can be linear or branched. Branched PEG derivatives, “star-PEGs” and multi-armed PEGs are contemplated by the present disclosure. Specific embodiments PEGs useful in the practice of the present invention include a 10 kDa linear PEG-aldehyde (e.g., Sunbright® ME-100AL, NOF America Corporation, One North Broadway, White Plains, NY 10601 USA), 10 kDa linear PEG-NHS ester (e.g., Sunbright® ME-100CS, Sunbright® ME-100AS, Sunbright® ME-100GS, Sunbright® ME-100HS, NOF), a 20 kDa linear PEG-aldehyde (e.g. Sunbright® ME-200AL, NOF, a 20 kDa linear PEG-NHS ester (e.g., Sunbright® ME-200CS, Sunbright® ME-200AS, Sunbright® ME-200GS, Sunbright® ME-200HS, NOF), a 20 kDa 2-arm branched PEG-aldehyde the 20 kDA PEG-aldehyde comprising two 10 kDA linear PEG molecules (e.g., Sunbright® GL2-200AL3, NOF), a 20 kDa 2-arm branched PEG-NHS ester the 20 kDA PEG-NHS ester comprising two 10 kDA linear PEG molecules (e.g., Sunbright® GL2-200TS, Sunbright® GL200GS2, NOF), a 40 kDa 2-arm branched PEG-aldehyde the 40 kDA PEG-aldehyde comprising two 20 kDA linear PEG molecules (e.g., Sunbright® GL2-400AL3), a 40 kDa 2-arm branched PEG-NHS ester the 40 kDA PEG-NHS ester comprising two 20 kDA linear PEG molecules (e.g., Sunbright® GL2-400AL3, Sunbright® GL2-400GS2, NOF), a linear 30 kDa PEG-aldehyde (e.g., Sunbright® ME-300AL) and a linear 30 kDa PEG-NHS ester.

Pegylation most frequently occurs at the α-amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

The PEG can be bound to an IL-10 polypeptide of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide. Another activated polyethylene glycol which can be bound to a free amino group is 2,4-bis(O-methoxypolyethyleneglycol)-6-chloro-s-triazine, which can be prepared by reacting polyethylene glycol monomethyl ether with cyanuric chloride. The activated polyethylene glycol which is bound to the free carboxyl group includes polyoxyethylenediamine.

Conjugation of one or more of the IL-10 polypeptide sequences of the present disclosure to PEG having a spacer can be carried out by various conventional methods. For example, the conjugation reaction can be carried out in solution at a pH of from 5 to 10, at temperature from 4 °C to room temperature, for 30 minutes to 20 hours, utilizing a molar ratio of reagent to protein of from 4:1 to 30:1. Reaction conditions can be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH≥7), and longer reaction time tend to increase the number of PEGs attached. Various means known in the art can be used to terminate the reaction. In some embodiments, the reaction is terminated by acidifying the reaction mixture and freezing at, e.g., −20 °C. Pegylation of various molecules is discussed in, for example, U.S. Pat. Nos. 5,252,714; 5,643,575; 5,919,455; 5,932,462; and 5,985,263. PEG-IL-10 is described in, e.g., U.S. Pat. No. 7,052,686. Specific reaction conditions contemplated for use herein are set forth in the Experimental section.

Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

Conjugation of one or more of the polypeptide sequences of the present disclosure to PEG having a spacer can be carried out by various conventional methods. For example, the conjugation reaction can be carried out in solution at a pH of from 5 to 10, at temperature from 4 °C to room temperature, for 30 minutes to 20 hours, utilizing a molar ratio of reagent to protein of from 4:1 to 30:1. Reaction conditions can be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH≥7), and longer reaction time tend to increase the number of PEGs attached. Various means known in the art can be used to terminate the reaction. In some embodiments, the reaction is terminated by acidifying the reaction mixture and freezing at, e.g., −20 °C. Pegylation of various molecules is discussed in, for example, U.S. Pat. Nos. 5,252,714; 5,643,575; 5,919,455; 5,932,462; and 5,985,263. PEG-IL-10 is described in, e.g., U.S. Pat. No. 7,052,686.

Although the present disclosure contemplates the synthesis of pegylated IL-10 by any means known to the skilled artisan, the following provides several alternative synthetic schemes for producing mono-PEG-IL-10 and a mix of mono-/di-PEG-IL-10 is meant to be illustrative only. While both mono-PEG-IL-10 and a mix of mono-/di-PEG-IL-10 have many comparable properties, a mix of selectively pegylated mono- and di-PEG-IL-10 improves the yield of the final pegylated product (see, e.g., U.S. Pat. No. 7,052,686 and US Pat. Publn. No. 2011/0250163). In addition to leveraging her own skills in the production and use of PEGs (and other drug delivery technologies) suitable in the practice of the present disclosure, the skilled artisan is also familiar with many commercial suppliers of PEG-related technologies (and other drug delivery technologies). By way of example, NOF America Corp (Irvine, CA) supplies mono-functional Linear PEGs, bi-functional PEGs, multi-arm PESs, branched PEGs, heterofunctional PEGs, forked PEGs, and releasable PEGs; and Parchem (New Rochelle, NY) is a global distributor of PEG products and other specialty raw materials.

Exemplary PEG-IL-10 Synthetic Scheme No. 1. IL-10 is dialyzed against 10 mM sodium phosphate pH 7.0, 100 mM NaCl. The dialyzed IL-10 is diluted 3.2 times to a concentration of about 0.5 to 12 mg/mL using the dialysis buffer. Prior to the addition of the linker, SC-PEG-12K (Delmar Scientific Laboratories, Maywood, Ill.), one volume of 100 mM Nα-tetraborate at pH 9.1 is added into 9 volumes of the diluted IL-10 to raise the pH of the IL-10 solution to 8.6. The SC-PEG-12K linker is dissolved in the dialysis buffer and the appropriate volume of the linker solution (1.8 to 3.6 mole linker per mole of IL-10) is added into the diluted IL-10 solution to initiate the pegylation reaction. The reaction is carried out at 5 °C in order to control the rate of the reaction, and the reaction solution is mildly agitated. When the mono-PEG-IL-10 yield, as determined by size exclusion HPLC (SE-HPLC), is close to 40%, the reaction is stopped by adding 1M glycine solution to a final concentration of 30 mM. The pH of the reaction solution is slowly adjusted to 7.0 using an HCl solution, and the reaction is 0.2 micron filtered and stored at −80 °C.

Exemplary PEG-IL-10 Synthetic Scheme No. 2. Mono-PEG-IL-10 is prepared using methoxy-PEG-aldehyde (PALD-PEG) as a linker (Inhale Therapeutic Systems Inc., Huntsville, AL; also available from NOF America Corp (Irvine, CA)). PALD-PEG can have molecular weights of 5 KDa, 12 KDa, or 20 KDa. IL-10 is dialyzed and diluted as described above, except the pH of the reaction buffer is between 6.3 and 7.5. Activated PALD-PEG linker is added to reaction buffer at a 1:1 molar ratio. Aqueous cyanoborohydride is added to the reaction mixture to a final concentration of 0.5 to 0.75 mM. The reaction is carried out at room temperature (18-25 °C) for 15-20 hours with mild agitation. The reaction is quenched with 1M glycine. Yields are analyzed by SE-HPLC. Mono-PEG-IL-10 is separated from unreacted IL-10, PEG linker and di-PEG-IL-10 by gel filtration chromatography and characterized by RP-HPLC and bioassay (e.g., stimulation of IL-10—responsive cells or cell lines).

Exemplary PEG-IL-10 Synthetic Scheme No. 3. IL-10 (e.g., rodent or primate) is dialyzed against 50 mM sodium phosphate, 100 mM sodium chloride pH ranges 5-7.4. A 1:1-1:7 molar ratio of 5K PEG-propyladehyde is reacted with IL-10 at a concentration of 1-12 mg/mL in the presence of 0.75-30 mM sodium cyanoborohydride. Alternatively the reaction can be activated with picoline borane in a similar manner. The reaction is incubated at 5-30 °C for 3-24 hours. The pH of the pegylation reaction is adjusted to 6.3, 7.5 mg/mL of hIL-10 is reacted with PEG to make the ratio of IL-10 to PEG linker 1:3.5. The final concentration of cyanoborohydride is ~25 mM, and the reaction is carried out at 15 °C for 12-15 hours. The mono- and di-PEG IL-10 are the largest products of the reaction, with the concentration of each at ~45-50% at termination. The reaction can be quenched using an amino acid such as glycine or lysine or, alternatively, Tris buffers. Multiple purification methods can be employed such as gel filtration, anion and cation exchange chromatographies, and size exclusion HPLC (SE-HPLC) to isolate the desired pegylated IL-10 molecules.

In some embodiments, the PEG-IL-10 agent is AM-0010. The term AM0010 refers to a recombinant human interleukin 10 (rHuIL-10) comprising an approximately 1:1 mixture of mono- and di-PEGylated rhIL-10 polypeptides and employing 5 kDa polyethylene glycol (PEG) attached via a linker to the N-terminus of the IL-10 polypeptide. AM0010 is a non-glycosylated homodimeric protein composed of two non-covalently associated rHuIL-10 polypeptide monomers, where each monomer is composed of 161 amino acids, including an N-terminal methionine not present in native human IL-10 polypeptide arising from direct expression recombinant bacterial production, each monomer comprising two intramolecular disulfide linkages, the first between cysteines at positions 13 and 109 and the second between cysteines at positions 63 and 115 of the 161 amino acid rHuIL-10 polypeptide (corresponding to cysteines at positions 12 and 108 and positions 62 and 114 of the naturally occurring hIL-10 polypeptide). AM0010 has been evaluated in multiple clinical trials and has been shown to well tolerated as a single agent at daily subcutaneous doses of up to 20 micrograms/kg at which does objective responses in renal cell carcinoma (RCC, 25% ORR), uveal melanoma and a CR in Cutaneous T-cell lymphoma with durable responses up to 2.5 years and prolonged stable disease in CRC and PDAC were observed.

### G. Glycosylated IL-10

In one embodiment of the invention, the modified IL-10 agent is a glycosylated IL-10. For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present. Glycosylation can dramatically affect the physical properties (e.g., solubility) of polypeptides such as IL-10 and can also be important in protein stability, secretion, and subcellular localization. Glycosylated polypeptides can also exhibit enhanced stability or can improve one or more pharmacokinetic properties, such as half-life. In addition, solubility improvements can, for example, enable the generation of formulations more suitable for pharmaceutical administration than formulations comprising the non-glycosylated polypeptide.

Addition of glycosylation sites can be accomplished by altering the amino acid sequence of the IL-10 polypeptide. The alteration to the IL-10 polypeptide can be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type can be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, can confer acidic properties to the glycoprotein. A particular embodiment of the present disclosure comprises the generation and use of N-glycosylation variants. Examples of IL-10 polypeptides comprising modified amino acid sequences to incorporate glycosylation site are provided in, for example, Van Vlasselaer, et al., United States Patent Application Publication No. US20160068583 A1 published Mar. 10, 2016. The IL-10 polypeptide sequences of the present disclosure can optionally be altered through changes at the nucleic acid level, particularly by mutating the nucleic acid encoding the polypeptide at preselected bases such that codons are generated that will translate into the desired amino acids to facilitate the introduction of glycosylation sites.

### H. Polysialated IL-10

In one embodiment of the invention, the modified IL-10 agent is polysialated IL-10. The term “polysialylation” refers to the conjugation of polypeptides to the naturally occurring, biodegradable α-(2→8) linked polysialic acid (“PSA”) in order to improve the polypeptides' stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2):125-30). Various techniques for site-specific polysialylation are available (see, e.g., T. Lindhout, et al. (2011) PNAS 108(18)7397-7402.

### I. IL-10 Fusion Proteins

In one embodiment of the invention, the modified IL-10 agent is conjugated to albumin referred to herein as an “IL-10 albumin fusion.” The term “albumin” as used in the context IL-10 albumin fusions include albumins such as human serum albumin (HSA), cyno serum albumin, and bovine serum albumin (BSA). According to the present disclosure, albumin can be conjugated to a IL-10 polypeptide (e.g., a polypeptide described herein) at the carboxyl terminus, the amino terminus, both the carboxyl and amino termini, and internally (see, e.g., U.S. Pat. Nos. 5,876,969 and 7,056,701). In the HSA-IL-10 polypeptide conjugates contemplated by the present disclosure, various forms of albumin can be used, such as albumin secretion pre-sequences and variants thereof, fragments and variants thereof, and HSA variants. Such forms generally possess one or more desired albumin activities. In additional embodiments, the present disclosure involves fusion proteins comprising an IL-10 polypeptide fused directly or indirectly to albumin, an albumin fragment, and albumin variant, etc., wherein the fusion protein has a higher plasma stability than the unfused drug molecule and/or the fusion protein retains the therapeutic activity of the unfused drug molecule. In some embodiments, the indirect fusion is accomplished by a linker, such as a peptide linker or modified version thereof.

Alternatively, the IL-10 albumin fusion comprises IL-10 polypeptides that are fusion proteins which comprise an albumin binding domain (ABD) polypeptide sequence and an IL-10 polypeptide. As alluded to above, fusion proteins which comprise an albumin binding domain (ABD) polypeptide sequence and an IL-10 polypeptide can, for example, be achieved by genetic manipulation, such that the nucleic acid coding for HSA, or a fragment thereof, is joined to the nucleic acid coding for the one or more IL-10 polypeptide sequences.

Additional suitable components and molecules for conjugation to an IL-10 agent include, for example, thyroglobulin; tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

The present disclosure contemplates conjugation of one or more additional components or molecules at the N- and/or C-terminus of a polypeptide sequence, such as another polypeptide (e.g., a polypeptide having an amino acid sequence heterologous to the subject polypeptide), or a carrier molecule. Thus, an exemplary polypeptide sequence can be provided as a conjugate with another component or molecule.

An IL-10 polypeptide can also be conjugated to large, slowly metabolized macromolecules such as proteins; polysaccharides, such as sepharose, agarose, cellulose, or cellulose beads; polymeric amino acids such as polyglutamic acid, or polylysine; amino acid copolymers; inactivated virus particles; inactivated bacterial toxins such as toxoid from diphtheria, tetanus, cholera, or leukotoxin molecules; inactivated bacteria; and dendritic cells. Such conjugated forms, if desired, can be used to produce antibodies against a polypeptide of the present disclosure.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

In certain embodiments, the amino- or carboxyl-terminus of an IL-10 polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product can require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

The present disclosure contemplates the use of other modifications of IL-10 agents to improve one or more properties. Examples include hesylation, various aspects of which are described in, for example, U.S. Patent Appln. Nos. 2007/0134197 and 2006/0258607, and IL-10 polypeptide fusion molecules comprising SUMO as a fusion tag (LifeSensors, Inc.; Malvern, PA).

The present disclosure also contemplates IL-10 agents wherein the IL-10 polypeptide is a fusion protein of an IL-10 polypeptide and one or more PEG mimetics. Polypeptide PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, CA). IL-10 agents comprising fusion proteins of such polypeptide sequences may be generated by recombinant means by expression of a nucleic acid sequence encoding this fusion protein obviating the need for additional conjugation step during the manufacturing process. Moreover, established molecular biology techniques enable control of the side chain composition of the polypeptide chains, allowing optimization of immunogenicity and manufacturing properties.

Linkers and their use have been described above. Any of the foregoing components and molecules used to modify the polypeptide sequences of the present disclosure can optionally be conjugated to an IL-10 agent or IL-10 polypeptide via a linker. Suitable linkers include “flexible linkers” which are generally of sufficient length to permit some movement between the modified polypeptide sequences and the linked components and molecules. The linker molecules are generally about 6-50 atoms long. The linker molecules can also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Examples of flexible linkers include glycine polymers (G)n, glycine-serine polymers (for example, (GS)ₙ, GSGGSₙ (SEQ ID NO: 16) and GGGSₙ (SEQ ID NO:17), where n is an integer of at least one), glycine-alanine polymers, alanine-serine polymers, and other flexible linkers. Glycine and glycine-serine polymers are relatively unstructured, and therefore can serve as a neutral tether between components.

Further examples of flexible linkers include glycine polymers (G)ₙ, glycine-alanine polymers, alanine-serine polymers, glycine-serine polymers (for example, (GₘSₒ)ₙ, (GSGGS)ₙ (SEQ ID NO:18), (GₘSₒGₘ)ₙ (SEQ ID NO:19), (GₘSₒGₘSₒGₘ)ₙ (SEQ ID NO:220), (GSGGSₘ)ₙ (SEQ ID NO:21), (GSGSₘG)ₙ (SEQ ID NO:22) and (GGGSₘ)ₙ (SEQ ID NO:23), and combinations thereof, where m, n, and o are each independently selected from an integer of at least 1 to 20, e.g., 1-18, 2-16, 3-14, 4-12, 5-10, 1, 2, 3, 4, 5, 6, 7, 8, 9, or 10), and other flexible linkers. Glycine and glycine-serine polymers are relatively unstructured, and therefore may serve as a neutral tether between components. Examples of flexible linkers include, but are not limited to GGSG (SEQ ID NO:24), GGSGG (SEQ ID NO:25), GSGSG (SEQ ID NO:26), GSGGG (SEQ ID NO:27), GGGSG (SEQ ID NO:28), and GSSSG (SEQ ID NO:29).

Additional flexible linkers include glycine polymers (G)ₙ or glycine-serine polymers (e.g., (GS)ₙ, (GSGGS)ₙ (SEQ ID NO:16), (GGGS)ₙ (SEQ ID NO:17) and (GGGGS)ₙ (SEQ ID NO:30), where n=1 to 50, for example, 1, 2, 3, 4, 5, 6, 7, 8, 9, 10, 10-20, 20-30, 30-50. Exemplary flexible linkers include, but are not limited to, GGGS (SEQ ID NO:31), GGGGS (SEQ ID NO:32), GGSG (SEQ ID NO:33), GGSGG (SEQ ID NO:34), GSGSG (SEQ ID NO:35), GSGGG (SEQ ID NO:36), GGGSG (SEQ ID NO:37), and GSSSG (SEQ ID NO:38). A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein. As described herein, the heterologous amino acid sequence may be a signal sequence and/or a fusion partner, such as, albumin, Fc sequence, and the like.

### J. Chimeric Antigen Receptors

CARs useful in the practice of the present invention are prepared in accordance with principles well known in the art. See e.g., Eshhaar et al. U.S. Pat. No. 7,741,465 B1 issued Jun. 22, 2010; Sadelain, et al (2013) Cancer Discovery 3(4):388-398 (The basic principles of chimeric antigen receptor (CAR) design); Jensen and Riddell (2015) Current Opinions in Immunology 33:9-15 (Designing chimeric antigen receptors to effectively and safely target tumors); Gross, et al. (1989) PNAS(USA) 86(24):10024-10028 (Expression of immunoglobulin-T-cell receptor chimeric molecules as functional receptors with antibody-type specificity); Curran, et al. (2012) J Gene Med 14(6):405-15. Considerations regarding the construction of the CAR and of the functional domains thereof in the context of the present invention are discussed below.

CAR-T cell therapy products have been approved for commercial use in the United States by the United States Food and Drug Administration which are amenable to use in accordance with the teaching of this disclosure. Examples of commercially available CAR-T cell products that may be used in conjunction with the methods and compositions described herein include axicabtagene ciloleucel (marketed as Yescarta® commercially available from Gilead Pharmaceuticals) and tisagenlecleucel (marketed as Kymriah® commercially available from Novartis).

### (a) Signal Sequence;

The CAR of the present invention comprises a signal peptide to facilitate surface display of the ARD (see below). In the practice of the present invention any eukaryotic signal peptide sequence may be employed. The signal peptide may be derived from native signal peptides of surface expressed proteins. In one embodiment of the invention, the signal peptide of the CAR is the signal peptide selected from the group consisting of human serum albumin signal peptide, prolactin albumin signal peptide, the human 1L2 signal peptide, human trypsinogen-2, human CD-5, the human immunoglobulin kappa light chain, human azurocidin, Gaussia luciferase and functional derivatives thereof. Particular amino acid substitutions to increase secretion efficiency using signal peptides are described in Stern, et al. (2007) Trends in Cell and Molecular Biology 2:1-17 and Kober, et al. (2013) Biotechnol Bioeng. 1110(4):1164-73. Alternatively, the signal peptide may be a synthetic sequence prepared in accordance established principles. See e.g., Nielsen, et al. (1997) Protein Engineering 10(1):1-6 (Identification of prokaryotic and eukaryotic signal peptides and prediction of their cleavage sites); Bendtsen, et al (2004) J. Mol. Biol 340(4):783-795 (Improved Prediction of Signal Peptides SignalP 3.0); Petersen, et al (2011) Nature Methods 8:785-796 (Signal P 4.0; discriminating signal peptides from transmembrane regions).

### (b) Extracellular Antigen Recognition Domain

The CAR of the present invention further comprises an extracellular antigen recognition domain (“ARD”) that specifically binds to an antigen expressed on the surface of a target cell. The ARD may be any single chain polypeptide specifically binds to an antigen expressed on the surface of a target cell. The choice of the antigen expressed on the surface of a target cell will dictate the design and selection of the ARD. In certain embodiments, the target cell population may comprise a tumor antigen. Vigneron, N. et al. ((15 Jul. 2013) Cancer Immunity 13:15) describe a database of T-cell-defined human tumor antigens containing over 400 tumor antigenic peptides. Examples of tumor antigens that may be targeted by the ARD of the CAR include one or more antigens selected from the group including, but not limited to, the HER2, MUC1, telomerase, PSA, CEA, VEGF, VEGF-R2, T1, CD19, CD20, CD22, ROR1, mesothelin, CD33/IL3Ra, c-Met, PSMA, Glycolipid F77, FAP, EGFRvIII, GD-2, NY-ESO-1 TCR, MAGE A3 TCR, 5T4, WT1, KG2D ligand (including MICA/B and UL3P-1, -2, -3, and -4), a Folate receptor (FRa), platelet-derived growth factor receptor A (also termed PDGFRα), and Wnt1 antigens.

In one embodiment, the ARD is a single chain Fv (ScFv). An ScFv is a polypeptide comprised of the variable regions of the immunoglobulin heavy and light chain of an antibody covalently connected by a peptide linker (Bird, et al. (1988) Science 242:423-426; Huston, et al. (1988) PNAS(USA) 85:5879-5883; S-z Hu, et al. (1996) Cancer Research, 56, 3055-3061; Ladner, U.S. Pat. No. 4,946,778 issued Aug. 7, 1990). The preparation of an anti-targeting antigen ScFv proceeds by generating a monoclonal antibody against the targeting antigen for from which the anti-targeting antigen ScFv is derived. The generation of monoclonal antibodies and isolation of hybridomas is a technique well known to those of skill in the art. See e.g. Monoclonal Antibodies: A Laboratory Manual, Second Edition, Chapter 7 (E. Greenfield, Ed. 2014 Cold Spring Harbor Press). Immune response may be enhanced through co-administration of adjuvants well known in the art such as alum, aluminum salts, or Freund's, SP-21, etc. Antibodies generated may be optimized to select for antibodies possessing particular desirable characteristics through techniques well known in the art such as phage display and directed evolution. See, e.g. Barbas, et al. (1991) PNAS(USA) 88:7978-82; Ladner, et al. U.S. Pat. No. 5,223,409 issued Jun. 29, 1993; Stemmer, W. (1994) Nature 370:389-91; Garrard U.S. Pat. No. 5,821,047 issued Oct. 13, 1998; Camps, et al. (2003) PNAS(USA) 100(17): 9727-32; Dulbecco U.S. Pat. No. 4,593,002 issued Jun. 3, 1986; McCafferty U.S. Pat. No. 6,806,079 issued Oct. 19, 2004; McCafferty, U.S. Pat. No. 7,635,666 issued Dec. 22, 2009; McCafferty, U.S. Pat. No. 7,662,557 issued Feb. 16, 2010; McCafferty, U.S. Pat. No. 7,723,271 issued May 25, 2010; and/or McCafferty U.S. Pat. No. 7,732,377. The generation of ScFvs based on monoclonal antibody sequences is well known in the art. See, e.g. The Protein Protocols Handbook, John M. Walker, Ed. (2002) Humana Press Section 150 “Bacterial Expression, Purification and Characterization of Single-Chain Antibodies” Kipriyanov, S. In some embodiments, the ARD is derived from an anti-CD19 scFv, an anti-PSA scFv, an anti-CD19 scFv, an anti-HER2 scFv, an anti-CEA scFv, an anti-EGFR scFv, an anti-MUC1 scFv, an anti-HER2-neu scFv, an anti-VEGF-R2 scFv, an anti-T1 scFv, an anti-CD22 scFv, an anti-ROR1 scFv, an anti-mesothelin scFv, an anti-CD33/IL3Ra scFv, an anti-c-Met scFv, an anti-PSMA scFv, an anti-Glycolipid F77 scFv, an anti-FAP scFv, an anti-EGFRvIII scFv, an anti-GD-2 scFv, an anti-NY-ESO-1 scFv, an anti-MAGE scFv, an anti-A3 scFv, an anti-5T4 scFv, an anti-WT1 scFv, or an anti-Wnt1 scFv.

In another embodiment, the ARD is a single domain antibody obtained through immunization of a camel or llama with a target cell derived antigen. See, e.g. Muyldermans, S. (2001) Reviews in Molecular Biotechnology 74: 277-302.

Alternatively, the ARD may be generated wholly synthetically through the generation of peptide libraries and isolating compounds having the desired target cell antigen binding properties. Such techniques are well known in the scientific literature. See, e.g. Wigler, et al. U.S. Pat. No. 6,303,313 B1 issued Nov. 12, 1999; Knappik, et al., U.S. Pat. No. 6,696,248 B1 issued Feb. 24, 2004, Binz, et al. (2005) Nature Biotechnology 23:1257-1268; Bradbury, et al. (2011) Nature Biotechnology 29:245-254.

In addition to the ARD having affinity for the target cell expressed antigen, the ARD may also have affinity for additional molecules. For example, an ARD of the present invention may be bi-specific, i.e. have capable of providing for specific binding to a first target cell expressed antigen and a second target cell expressed antigen. Examples of bivalent single chain polypeptides are known in the art. See, e.g. Thirion, et al. (1996) European J. of Cancer Prevention 5(6):507-511; DeKruif and Logenberg (1996) J. Biol. Chem 271(13)7630-7634; and Kay, et al. United States Patent Application Publication Number 2015/0315566 published Nov. 5, 2015.

In an alternative embodiment, the CAR or the ARD of the CAR may be derived from the TCR of a clone induced in response to immunotherapy. Methods for the identification of novel tumor specific TCR sequences and the incorporation such sequences into the production of CAR T-cells comprising these sequences are described in Mumm, et al. PCT/US2017/012882 published as WO2017/123557A1 on Jul. 20, 2017 the entire teaching of which is herein incorporated by reference. Briefly, IL-10 agent therapy results in the induction of disease antigen-specific CD8+ T-cells into the periphery of a patient following administration of the IL-10 agent to the patient. After the patient has received the IL-10 agent therapy for a period of time, a tissue sample containing lymphocytes, e.g., a peripheral blood sample containing peripheral blood lymphocytes (PBLs), may be collected from the patient by conventional procedures such as leukapheresis. After collecting the tissue sample, nucleic acids in the sample are analyzed by sequencing to obtain TCR sequences (e.g., encoding a variable alpha (Vα) TCR polypeptide and/or nucleic acids encoding a variable beta (Vβ) TCR polypeptide). The sequencing reads may be analyzed to obtain an estimate of the abundance of nucleic acids encoding the Vα TCR polypeptide and/or nucleic acids encoding the Vβ TCR polypeptide for TCRs expressed on CD8+ T-cells, i.e., functionally present on a cell surface of antigen-specific T-cells, in the sample. By comparing the abundance of nucleic acids encoding the Vα TCR polypeptide and/or nucleic acids encoding the Vβ TCR polypeptide for TCRs expressed on CD8+ T-cells in the sample with the abundance of the nucleic acids encoding the Vα TCR polypeptide and/or nucleic acids encoding Vβ TCR polypeptide in a reference sample at an earlier time point during IL-10 agent therapy, it is possible to identify a particular T-cell population expressing an antigen-specific TCR (defined by the α chain and β chain TCR pair sequences) has clonally expanded, clonally contracted, or has been newly generated in response to the IL-10 agent therapy. After sequencing nucleic acids encoding paired alpha and beta chain of a TCR expressed on the surface of CD8+ T-cells, e.g., isolated CD8+ T-cells, the amino acid sequence of the alpha and beta chains, including the CDR regions of each chain, may be determined. These TCR pair amino acid sequences may be employed to generate recombinant disease antigen-specific CAR-T cells by transducing nucleic acid constructs encoding full-length α chain and β chain TCR pair amino sequences, or chimeric antigen receptor containing the variable regions of the α chain and β chain TCR pair amino sequences. Such disease antigen-specific CAR-T cells may then be administered to a suitable patient in need of treatment for the disease, including the patient from which the novel TCR sequence was isolated as that CAR-T cell would be particularly selected for activity against that subject's tumor cells. Methods for the isolation of neoantigen induced T-cells are described in Cohen, et al. (2015) Journal of Clinical Investigation 125(10):3981-3991. Such patient derived sequences are particularly useful in the practice of the present invention as these novel T-cell clones induced in response to immunotherapy, particularly IL-10 therapy, comprise TCRs having selected affinity for a population of tumor cells present in the subject and therefore would be expected to provide enhanced specificity and targeting efficiency relative to “generic” tumor antigens.

### (c) Transmembrane Domain:

CARs useful in the practice of the present invention further provide a transmembrane spanning domain linking the anti-targeting antigen ARD (or spacer if included) to the intracellular domain of the CAR. The transmembrane spanning domain is comprised of any sequence which is thermodynamically stable in a eukaryotic cell membrane. Transmembrane spanning domains useful in construction of CARs useful in the practice of the present invention are comprised of approximately 20 amino acids favoring the formation having an alpha-helical secondary structure. The transmembrane spanning domain may be derived from the transmembrane domain of a naturally occurring membrane spanning protein.

Alternatively, the transmembrane domain may be synthetic. In designing synthetic transmembrane domains, amino acids favoring alpha-helical structures are preferred. Amino acids favoring the formation of alpha-helices are well known in the art. See e.g., Pace, et al. (1998) Biophysical Journal 75:422-427.

### (d) Intracellular Signaling Domain

The intracellular domain of the CAR comprises one or more intracellular signal transduction domains (e.g. the CD3 ζ-chain). In one embodiment, the intracellular signal domains comprise the cytoplasmic sequences of the T-cell receptor (TCR) and co-receptors that initiate signal transduction following antigen receptor engagement and functional derivatives and sub-fragments thereof. Additionally, or alternatively, the cytoplasmic domain of the CAR may comprise one or more intracellular signaling domains. Examples of intracellular signaling domains include but are not limited to the cytoplasmic domain of CD27, CD28, the cytoplasmic domain of CD137 (also referred to as 4-1BB and TNFRSF9), the cytoplasmic domain of CD278 (also referred to as ICOS), p110α, β, or δ catalytic subunit of PI3 kinase), CD3 ζ-chain, cytoplasmic domain of CD134 (also referred to as OX40 and TNFRSF4). FcεR1γ and β chains, MB1 (Igα) chain, B29 (Igβ) chain, etc.), human CD3 zeta chain, CD3 polypeptides (δ, Δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. In one embodiment of the invention, the intracellular signal transduction domain of the CAR is CD3 ζ-chain. In another embodiment of the invention, the intracellular signal transduction domain of the CAR comprises CD3 ζ-chain and the cytoplasmic domain of CD28. In another embodiment of the invention, the intracellular signal transduction domain of the CAR is a trimeric structure comprising the CD3 ζ-chain, the cytoplasmic domain S of CD28 and OX40. In one embodiment, the intracellular signal transduction domain comprises the signaling domain of CD3-zeta and the signaling domain of CD28. In another embodiment, the intracellular signal transduction domain comprises the signaling domain of CD3 ζ and the signaling domain of CD137. In another embodiment, the cytoplasmic domain comprises the signaling domain of CD3-zeta and the signaling domain of CD28 and CD137. The intracellular domain may, in addition to one signaling domain may also provide one or more “co-stimulatory domains” (CSDs). The co-stimulatory domain refers to the portion of the CAR which enhances the proliferation, survival or development of memory cells. In some embodiments of the present disclosure, the CSD comprises one or more of members of the TNFR superfamily, CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or combinations thereof. The ordinarily skilled artisan is aware of other co-stimulatory domains that may be used in conjunction with the teachings of the present disclosure.

There has been a relatively rapid progression of CAR-T cell therapy (see generally, US Patent Application Publication No. 20150038684), much of which has focused on the nature of the intracellular signaling domain. So-called “first generation CARs” were directed to fusion of antigen-recognition domains to the CD3ζ activation chain of the T-cell receptor (TCR) complex. While these first-generation CARs induced T-cell effector function in vitro, in vivo efficacy was largely limited by their poor antitumor efficacy. Evolution of CAR technology resulted in “second generation CARs,” which include the CD3ζ activation chain in tandem with one CSD, examples of which include intracellular domains from CD28 or a variety of TNF receptor family molecules such as 4-1BB (41BB, CD137) and OX40 (CD134). “Third generation CARs” have been developed that include two costimulatory signals in addition to the CD3ζ activation chain, the CSDs most commonly being from CD28 and 4-1BB. Second and third generation CARs dramatically improved antitumor efficacy. The increased potency of second and third generation CARs, coupled with the possibility that the antigen-target for the CAR-T cell is also expressed on non-target cells, has also resulted in the increased risk of severe toxicities. (See, e.g., Carpenito et al. (2009) Proc Natl Acad Sci USA 106(9):3360-65; Grupp et al. (2013) N Engl J Med 368(16):1509-18) and consequently, the use of such second and third generation CAR-Ts should be evaluated at lower doses than those typically associated with first generation CAR-Ts.

In some embodiments of the invention, the intracellular signaling domain comprises a polypeptide of the following domains arranged amino to carboxy in the following sequence:
CD3ζ
CD28-41BB-CD3ζ,
CD28-CD3ζ
CD28-OX40-CD3ζ
CD28-41BB-CD3ζ
OX40-CD3ζ
OX40-CD28-CD3ζ
41BB-CD3ζ
ICOS-CD3ζ
ICOS-41BB-CD3ζ
41BB-ICOS-CD3ζ
41BB-OX40-CD3ζ, and
41BB-CD28-CD3ζ.

### (e) Linkers

CARs useful in the practice of the present invention may optionally include one or more polypeptide spacers linking the domains of the CAR, in particular the linkage between the ARD to the transmembrane spanning domain of the CAR. Although not an essential element of the CAR structure, the inclusion of a spacer domain is generally considered desirable to facilitate antigen recognition by the ARD. Moritz and Groner (1995) Gene Therapy 2(8) 539-546. As used in conjunction with the CAR-T cell technology described herein, the terms “linker”, “linker domain” and “linker region” refer to an oligo- or polypeptide region from about 1 to 100 amino acids in length, which links together any of the domains/regions of the CAR of the disclosure. Linkers may be composed of flexible residues like glycine and serine so that the adjacent protein domains are free to move relative to one another. Certain embodiments comprise the use of linkers of longer length when it is desirable to ensure that two adjacent domains do not sterically interfere with each another. In some embodiments, the linkers are non-cleavable, while in others they are cleavable (e.g., 2A linkers (for example T2A)), 2A-like linkers or functional equivalents thereof, and combinations of the foregoing. There is no particular sequence of amino acids that is necessary to achieve the spacer function but the typical properties of the spacer are flexibility to enable freedom of movement of the ARD to facilitate targeting antigen recognition. Similarly, it has been found that there is substantial leniency in spacer length while retaining CAR function. Jensen and Riddell (2014) Immunol. Review 257(1) 127-144. Sequences useful as spacers in the construction of CARs useful in the practice of the present invention include but are not limited to the hinge region of IgG1, the immunoglobulin1CH2-CH3 region, IgG4 hinge-CH2-CH3, IgG4 hinge-CH3, and the IgG4 hinge. The hinge and transmembrane domains may be derived from the same molecule such as the hinge and transmembrane domains of CD8-alpha. Imai, et al. (2004) Leukemia 18(4):676-684. Embodiments of the present disclosure are contemplated wherein the linkers include the picornaviral 2A-like linker, CHYSEL sequences of porcine teschovirus (P2A), thosea asigna virus (T2A), or combinations, variants and functional equivalents thereof. In still further embodiments, the linker sequences comprise Asp-Val/Ile-Glu-X-Asn-Pro-Gly^{(2A)}-pro^{(2B)} motif, which results in cleavage between the 2A glycine and the 2B proline.

In some embodiments of the invention, the CAR is a polypeptide comprising the following functional domains, which may provide intervening or spacer sequences, arranged amino to carboxy terminus as follows:
anti-CD20-CD3ζ
anti-CD20-CD28-41BB-CD3ζ,
anti-CD20-CD28-CD3ζ
anti-CD20-CD28-OX40-CD3ζ
anti-CD20-CD28-41BB-CD3ζ
anti-CD20-OX40-CD3ζ
anti-CD20-OX40-CD28-CD3ζ
anti-CD20-41BB-CD3ζ
anti-CD20-ICOS-CD3ζ
anti-CD20-ICOS-41BB-CD3ζ
anti-CD20-41BB-ICOS-CD3ζ
anti-CD20-41BB-OX40-CD3ζ,
anti-CD20-41BB-CD28-CD3ζ.
anti-HER2-CD3ζ
anti-HER2-CD28-41BB-CD3ζ,
anti-HER2-CD28-CD3ζ
anti-HER2-CD28-OX40-CD3ζ
anti-HER2-CD28-41BB-CD3ζ
anti-HER2-OX40-CD3ζ
anti-HER2-OX40-CD28-CD3ζ
anti-HER2-41BB-CD3ζ
anti-HER2-ICOS-CD3ζ
anti-HER2-ICOS-41BB-CD3ζ
anti-HER2-41BB-ICOS-CD3ζ
anti-HER2-41BB-OX40-CD3ζ,
anti-HER2-41BB-CD28-CD3ζ.
anti-CEA-CD3ζ
anti-CEA-CD28-41BB-CD3ζ,
anti-CEA-CD28-CD3ζ
anti-CEA-CD28-OX40-CD3ζ
anti-CEA-CD28-41BB-CD3ζ
anti-CEA-OX40-CD3ζ
anti-CEA-OX40-CD28-CD3ζ
anti-CEA-41BB-CD3ζ
anti-CEA-ICOS-CD3ζ
anti-CEA-ICOS-41BB-CD3ζ
anti-CEA-41BB-ICOS-CD3ζ
anti-CEA-41BB-OX40-CD3ζ,
anti-CEA-41BB-CD28-CD3ζ.
anti-VEGF-CD3ζ
anti-VEGF-CD28-41BB-CD3ζ,
anti-VEGF-CD28-CD3ζ
anti-VEGF-CD28-OX40-CD3ζ
anti-VEGF-CD28-41BB-CD3ζ
anti-VEGF-OX40-CD3ζ
anti-VEGF-OX40-CD28-CD3ζ
anti-VEGF-41BB-CD3ζ
anti-VEGF-ICOS-CD3ζ
anti-VEGF-ICOS-41BB-CD3ζ
anti-VEGF-41BB-ICOS-CD3ζ
anti-VEGF-41BB-OX40-CD3ζ,
anti-VEGF-41BB-CD28-CD3ζ.
anti-CD19-CD3ζ
anti-CD19-CD28-41BB-CD3ζ,
anti-CD19-CD28-CD3ζ
anti-CD19-CD28-OX40-CD3ζ
anti-CD19-CD28-41BB-CD3ζ
anti-CD19-OX40-CD3ζ
anti-CD19-OX40-CD28-CD3ζ
anti-CD19-41BB-CD3ζ
anti-CD19-ICOS-CD3ζ
anti-CD19-ICOS-41BB-CD3ζ
anti-CD19-41BB-ICOS-CD3ζ
anti-CD19-41BB-OX40-CD3ζ,
anti-CD19-41BB-CD28-CD3ζ.
anti-EGFR-CD3ζ
anti-EGFR-CD28-41BB-CD3ζ,
anti-EGFR-CD28-CD3ζ
anti-EGFR-CD28-OX40-CD3ζ
anti-EGFR-CD28-41BB-CD3ζ
anti-EGFR-OX40-CD3ζ
anti-EGFR-OX40-CD28-CD3ζ
anti-EGFR-41BB-CD3ζ
anti-EGFR-ICOS-CD3ζ
anti-EGFR-ICOS-41BB-CD3ζ
anti-EGFR-41BB-ICOS-CD3ζ
anti-EGFR-41BB-OX40-CD3ζ, and
anti-EGFR-41BB-CD28-CD3ζ.

### K. CAR Expression Vectors

The preparation of CAR T-cells useful in the practice of the present invention is achieved by transforming isolated T-cells with an expression vector comprising a nucleic acid sequence encoding the CAR polyprotein described above.

Expression vectors for expression of the CAR in the T-cell may be viral vectors or non-viral vectors. The term “nonviral vector” refers to an autonomously replicating, extrachromosomal circular DNA molecule, distinct from the normal genome and nonessential for cell survival under nonselective conditions capable of effecting the expression of a coding sequence in the target cell. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, magnetic fields (electroporation)

In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are well known in the art including lipidic vector systems (Lee et al. (1997) Crit Rev Ther Drug Carrier Syst. 14:173-206); polymer coated liposomes (Marin et al., U.S. Pat. No. 5,213,804, issued May 25, 1993; Woodle, et al., U.S. Pat. No. 5,013,556, issued May 7, 1991); cationic liposomes (Epand et al., U.S. Pat. No. 5,283,185, issued Feb. 1, 1994; Jessee, J. A., U.S. Pat. No. 5,578,475, issued Nov. 26, 1996; Rose et al, U.S. Pat. No. 5,279,833, issued Jan. 18, 1994; Gebeyehu et al., U.S. Pat. No. 5,334,761, issued Aug. 2, 1994). The efficiency of expression CAR sequences in T-cells with non-viral vectors can be considerably increased by the use of transposon/transposase systems such as the so-called Sleeping Beauty (SB) transposon system (See. e.g., Geurts, et al. (2003) Mol Ther 8(1):108-117) and the piggyBac system (See, e.g. Manuri, et al. (2010) Human Gene Therapy 21(4):427-437) can be used to stably introduce non-viral vectors (e.g. plasmids) comprising nucleic acid sequences encoding anti-targeting antigen CAR into human T-cells.

In another embodiment, the expression vector may be a viral vector. As used herein, the term viral vector is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. A viral vector may be replication competent (e.g., substantially wild-type), conditionally replicating (recombinantly engineered to replicate under certain conditions) or replication deficient (substantially incapable of replication in the absence of a cell line capable of complementing the deleted functions of the virus). The viral vector can possess certain modifications to make it “selectively replicating,” i.e. that it replicates preferentially in certain cell types or phenotypic cell states, e.g., cancerous. Viral vector systems useful in the practice of the instant invention include, for example, naturally occurring or recombinant viral vector systems. Examples of viruses useful in the practice of the present invention include recombinantly modified enveloped or non-enveloped DNA and RNA viruses. For example, viral vectors can be derived from the genome of human or bovine adenoviruses, vaccinia virus, lentivirus, herpes virus, adeno-associated virus, human immunodeficiency virus, sindbis virus, and retroviruses (including but not limited to Rous sarcoma virus), and hepatitis B virus. Typically, genes of interest are inserted into such vectors to allow packaging of the gene construct, typically with accompanying viral genomic sequences, followed by infection of a sensitive host cell resulting in expression of the gene of interest (e.g. a targeting antigen). Additionally, the expression vector encoding the anti-targeting antigen CAR may also be an mRNA vector. When a viral vector system is to be employed for transfection, retroviral or lentiviral expression vectors are preferred to transfect T-cells due to an enhanced efficacy of gene transfer to T-cells using these systems resulting in a decreased time for culture of significant quantities of T-cells for clinical applications. In particular, gamma retroviruses a particularly preferred for the genetic modification of clinical grade T-cells and have been shown to have therapeutic effect. Pule, et al. (2008) Nature Medicine 14(11):1264-1270. Similarly, self-inactivating lentiviral vectors are also useful as they have been demonstrated to integrate into quiescent T-cells. June, et al. (2009) Nat Rev Immunol 9(10):704-716. Particular retroviral vectors useful in the expression of CAR sequences (and optional additional transgenes) are those described in Naldini, et al. (1996) In Vivo Gene Delivery and Stable Transduction of Nondividing Cells by a Lentiviral Vector, Science 272: 263-267; Naldini, et al. (1996) Efficient transfer, integration, and sustained long-term expression of the transgene in adult rat brains injected with a lentiviral vector, Proc. Natl. Acad. Sci. USA Vol. 93, pp. 11382-11388; Dull, et al. (1998) A Third-Generation Lentivirus Vector with a Conditional Packaging System, J. Virology 72(11):8463-8471; Milone, et al. (2009) Chimeric Receptors Containing CD137 Signal Transduction Domains Mediate Enhanced Survival of T Cells and Increased Antileukemic Efficacy In Vivo, Molecular Therapy 17(8):1453-1464; Kingsman, et al. U.S. Pat. No. 6,096,538 issued Aug. 1, 2000 and Kingsman, et al. U.S. Pat. No. 6,924,123 issued Aug. 2, 2005 herein incorporated by reference. In one embodiment of the invention, the CAR expression vector is a Lentivector® lentiviral vector available under license from Oxford Biomedica.

### L. Optional Transgenes Encoded and Expressed by the CAR Vector

The expression vector for the CAR may encode one or more polypeptides in addition to the targeting antigen. When expressing multiple polypeptides as in the practice of the present invention, each polypeptide may be operably linked to an expression control sequence (monocistronic) or multiple polypeptides may be encoded by a polycistronic construct where multiple nucleic acid sequences are operably linked to a single expression control sequence, optionally providing intervening sequences (e.g. IRES elements.

In one embodiment, the expression vector encoding the targeting antigen may optionally further encode one or more immunological modulators. Examples of immunological modulators useful in the practice of the present invention include but are not limited to cytokines. Examples of such cytokines are interleukins including but not limited to one more or of IL-1, IL-2, IL-3, IL-4, IL-12, IL-18, TNF-alpha, interferon alpha, interferon alpha-2b, interferon-beta, interferon-gamma, GM-CSF, MIP1-alpha, MIP1-beta, MIP3-alpha, TGF-beta and other suitable cytokines capable of modulating immune response. The expressed cytokines can be directed for intracellular expression or expressed with a signal sequence for extracellular presentation or secretion.

IL-12: In one embodiment, the vector further comprises nucleic acid sequences encoding polypeptide IL-12 agents, in one embodiment by providing the IL-12A(p35) and IL-12B(p40) coding sequences necessary to generate the IL-12 tetramer which is reported to provide enhanced antitumor efficacy in the context of CAR-T cell therapy (See, e.g. Pegram et al (2012) Blood 119(18):4133-4141; Yeku, et al (2017) Scientific Reports Vol. 7, Article number: 10541 Published online: 5 Sep. 2017).

IL15 Agents: In another embodiment, the vector further comprises nucleic acid sequences encoding polypeptide IL-15 agent. The term polypeptide IL-15 agent includes variants, analogs of the human IL-15 molecule. In another embodiment, the vector further comprises nucleic acid sequences encoding pre-pro-human IL-15 polypeptide (hIL15) having the sequence:

(SEQ ID NO: 39)
MRISKPHLRS ISIQCYLCLL LNSHFLTEAG IHVFILGCFS
AGLPKTEANW VNVISDLKKI EDLIQSMHID ATLYTESDVH
PSCKVTAMKC FLLELQVISL ESGDASIHDT VENLIILANN
SLSSNGNVTE SGCKECEELE EKNIKEFLQS FVHIVQMFIN
TS

In another embodiment, the vector further comprises nucleic acid sequences encoding pre-human IL-15 polypeptide (hIL15) having the sequence:

SEQ ID NO: 40)
MRISKPHLRS ISIQCYLCLL LNSHFLTEAN WVNVISDLKK
IEDLIQSMHI DATLYTESDV HPSCKVTAMK CFLLELQVIS
LESGDASIHD TVENLIILAN NSLSSNGNVT ESGCKECEEL
EEKNIKEFLQ SFVHIVQMFI NTS

In another embodiment, the vector further comprises nucleic acid sequences encoding pro-human IL-15 polypeptide (hIL15) having the sequence:

(SEQ ID NO: 41)
GIHVFILGCF SAGLPKTEAN WVNVISDLKK IEDLIQSMHI
DATLYTESDV HPSCKVTAMK CFLLELQVIS LESGDASIHD
TVENLIILAN NSLSSNGNVT ESGCKECEEL EEKNIKEFLQ
SFVHIVQMFI NTS

optionally providing an N-terminal methionyl residue when directly expressed without a leader sequence. In another embodiment, the vector further comprises nucleic acid sequences encoding mature human IL-15 polypeptide (hIL15) having the sequence:

(SEQ ID NO: 42)
NWVNVISDLK KIEDLIQSMH IDATLYTESD VHPSCKVTAM
KCFLLELQVI SLESGDASIH DTVENLIILA NNSLSSNGNV
TESGCKECEE LEEKNIKEFL QSFVHIVQMF INTS

optionally providing an N-terminal methionyl residue when directly expressed without a leader sequence. In a preferred practice of the invention, the IL-15 agent retains the disulfide linkages between cysteine residues 83-133 and 90-136 and/or is N-linked glycosylated GlcNAc at position 127.

Obtaining nucleic acid sequences encoding the foregoing polypeptide IL-15 agents is well known to those of skill in the art. See, e.g. Grabstein, et al. (1994) Cloning of a T cell growth factor that interacts with the beta chain of the interleukin-2 receptor, Science 264:965-968; Krause, et al. (1996) Genomic sequence and chromosomal location of the human interleukin-15 gene (IL15), Cytokine 8:667-674; and/or Tagaya, et al (1997) Generation of secretable and nonsecretable interleukin 15 isoforms through alternate usage of signal peptides, PNAS (USA) 94:14444-14449.

IL-2 Agents: In another embodiment, the vector further comprises nucleic acid sequences encoding polypeptide IL-2 agents. The term polypeptide IL-2 agent includes variants, analogs of the human IL-2 molecule. In another embodiment, the vector further comprises nucleic acid sequences encoding a pre-human IL-2 polypeptide (hIL2) having the sequence:

(SEQ ID NO: 43)
MYRMQLLSCI ALSLALVTNS APTSSSTKKT QLQLEHLLLD
LQMILNGINN YKNPKLTRML TFKFYMPKKA TELKHLQCLE
EELKPLEEVL NLAQSKNFHL RPRDLISNIN VIVLELKGSE
TTFMCEYADE TATIVEFLNR WITFCQSIIS TLT

In another embodiment, the vector further comprises nucleic acid sequences encoding the mature hIL-2 polypeptide having the sequence:

(SEQ ID NO: 44)
APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT

optionally providing an N-terminal methionyl residue when directly expressed without a leader sequence. In a preferred practice of the invention, the IL-2 agent retains the disulfide linkages between cysteine residues 78-125 and/or is glycosylated at position 23.

Obtaining nucleic acid sequences encoding the foregoing IL-2 agents is well known to those of skill in the art. See, e.g. Taniguchi, et al. (1983) Nature 302:315-310; Devos, et al (1983) Nucleic Acids Research 11:4307-4323; or Fujita, et al (1983) PNAS(USA) 80: 7347-7441.

IL-7 Agents: In another embodiment, the vector further comprises nucleic acid sequences encoding polypeptide IL-7 agents. The term polypeptide IL-7 agent includes variants, analogs of the human IL-7 molecule. In one embodiment, the vector further comprises nucleic acid sequences encoding a pre-human IL-7 polypeptide (hIL7) having the sequence:

(SEQ ID NO: 45)
MFHVSFRYIF GLPPLILVLL PVASSDCDIE GKDGKQYESV
LMVSIDQLLD SMKEIGSNCL NNEFNFFKRH ICDANKEGMF
LFRAARKLRQ FLKMNSTGDF DLHLLKVSEG TTILLNCTGQ
VKGRKPAALG EAQPTKSLEE NKSLKEQKKL NDLCFLKRLL
QEIKTCWNKI LMGTKEH

In another embodiment, the vector further comprises nucleic acid sequences encoding the mature hIL-7 polypeptide having the sequence:

(SEQ ID NO: 46)
DCDIEGKDGK QYESVLMVSI DQLLDSMKEI GSNCLNNEFN
FFKRHICDAN KEGMFLFRAA RKLRQFLKMN STGDFDLHLL
KVSEGTTILL NCTGQVKGRK PAALGEAQPT KSLEENKSLK
EQKKLNDLCF LKRLLQEIKT CWNKILMGTK EH

optionally providing an N-terminal methionyl residue when directly expressed without a leader sequence. In a preferred practice of the invention, the IL-7 agent retains the disulfide linkages between cysteine residues 27-166, 59-154 and 72-117 and/or is glycosylated at one or more of positions 95, 116, and/or 141. Obtaining nucleic acid sequences encoding the foregoing polypeptide IL-7 agents is well known to those of skill in the art.

IL-18 Agents: In another embodiment, the vector further comprises nucleic acid sequences encoding polypeptide IL-18 agents. The term polypeptide IL-18 agent includes variants, analogs of the human IL-18 molecule. In one embodiment, the polypeptide IL-18 agent is a precursor of isoform 1 of hIL-18 with a signal sequence having the amino acid sequence:

(SEQ ID NO: 47)
MAAEPVEDNC INFVAMKFID NTLYFIAEDD ENLESDYFGK
LESKLSVIRN LNDQVLFIDQ GNRPLFEDMT DSDCRDNAPR
TIFIISMYKD SQPRGMAVTI SVKCEKISTL SCENKIISFK
EMNPPDNIKD TKSDIIFFQR SVPGHDNKMQ FESSSYEGYF
LACEKERDLF KLILKKEDEL GDRSIMFTVQ NED

In another embodiment, the vector further comprises nucleic acid sequences encoding the mature hIL-18 isoform 1 polypeptide having the sequence:

(SEQ ID NO: 48)
YFGKLESKLS VIRNLNDQVL FIDQGNRPLF EDMTDSDCRD
NAPRTIFIIS MYKDSQPRGM AVTISVKCEK ISTLSCENKI
ISFKEMNPPD NIKDTKSDII FFQRSVPGHD NKMQFESSSY
EGYFLACEKE RDLFKLILKK EDELGDRSIM FTVQNED

In one embodiment, the polypeptide IL-18 agent is a precursor of isoform 2 (delta27-30 of the canonical sequence) of hIL-18 with a signal sequence having the amino acid sequence:

(SEQ ID NO: 49)
MAAEPVEDNC INFVAMKFID NTLYFIENLE SDYFGKLESK
LSVIRNLNDQ VLFIDQGNRP LFEDMTDSDC RDNAPRTIFI
ISMYKDSQPR GMAVTISVKC EKISTLSCEN KIISFKEMNP
PDNIKDTKSD IIFFQRSVPG HDNKMQFESS SYEGYFLACE
KERDLFKLIL KKEDELGDRS IMFTVQNED

Obtaining nucleic acid sequences encoding the foregoing polypeptide IL-18 agents is well known to those of skill in the art.

In one embodiment, in addition to an expression cassette for a targeting antigen, the expression vector further comprises expression cassettes comprising nucleic acid sequences encoding an IL-10 polypeptide, in particular an IL-10 peptide comprising a secretion leader sequence. Alternative to the use of multiple expression cassettes, the nucleic acid sequences encoding the CAR and IL-10 polypeptide may be encoded by a polycistronic construct, the expression cassette comprising the nucleic acid sequences CAR and IL-10 polypeptide employing sequences to facilitate expression of downstream coding sequences of the polycistronic constructing including but not limited to internal ribosome entry site (IRES) elements, the EF1a core promoter, or the nucleic acid sequence of foot and mouth disease virus protein 2A (FMVD2A) to facilitate co-expression in the target cell.

The expression vector may optionally provide an additional expression cassette comprising a nucleic acid sequence encoding a “rescue” gene. A “rescue gene” is a nucleic acid sequence, the expression of which renders the cell susceptible to killing by external factors or causes a toxic condition in the cell such that the cell is killed. Providing a rescue gene enables selective cell killing of transduced cells. Thus, the rescue gene provides an additional safety precaution when the constructs are incorporated into the cells of a mammalian subject to prevent undesirable spreading of transduced cells or the effects of replication competent vector systems. In one embodiment, the rescue gene is the thymidine kinase (TK) gene (see e.g. Woo, et al. U.S. Pat. No. 5,631,236 issued May 20, 1997 and Freeman, et al. U.S. Pat. No. 5,601,818 issued Feb. 11, 1997) in which the cells expressing the TK gene product are susceptible to selective killing by the administration of gancyclovir. Alternatively, the rescue gene may encode a known cell-surface antigen (e.g. CD20 or EGFR) enabling selective killing of the CAR-T cells by the administration of a molecule targeting such cells (e.g. rituximab (Rituxan®) for selective elimination of CD20 expressing cells or cetuximab (Erbitux®) for selective elimination of EGFR expressing cells).

In one embodiment, the expression vector may optionally provide an additional expression cassette comprising a nucleic acid sequence encoding a binding molecule against ITIM. In one embodiment, the expression vector may optionally provide an additional expression cassette comprising a nucleic acid sequence encoding a molecule which binds to an immunoreceptor tyrosine-based inhibition motif (ITIM) on the cytoplasmic domain of an inhibitory receptor of the immune system inhibiting its activity. An ITIM is a conserved sequence of amino acids typically of the sequence S/I/V/LxYxxI/V/L. When ITIM-possessing inhibitory receptors interact with their ligand, their ITIM motif is phosphorylate by Src kinase family enzymes faciliting their ability to recruit other enzymes such as phosphotyrosine phosphatases SHP-1 and/or SHP-2 or the SHIP inositol phosphatase called SHIP. These phosphatases downregulate the activity of molecules involved in cell signaling. Examples of molecules that bind such ITIM motifs are known in the art and may be used, for example, in the design of binding molecules (e.g. ScFvs) capable of intracellular expression from the CAR expression vector so as to inhibit the downregulation of immune functions mediated by phosphotyrosine phosphatases or inositol phosphatases including but not limited to one or more of SHP-1, SHP-2 and SHIP.

In an alternative embodiment, the expression vector may optionally provide an additional expression cassette comprising a nucleic acid sequence encoding a receptor and/or receptor subunits, particularly in the case of heteromultimeric receptors (e.g. IL-12). In particular embodiments, the receptor encoded by the vector is one or more of the receptors selected from the group consisting of the IL2 receptor, the IL7 receptor, the IL10 receptor, the IL12 receptor, the IL17 receptor, the IL18 receptor, and functional analogs thereof. In some embodiments, the vector further comprises nucleic acid sequences encoding one or more of the foregoing receptors with a secretion leader sequence to facilitate display of the vector on the surface of the CAR T-cell.

### M. Obtaining CAR-T Cell Source Cells

Chimeric antigen receptor T-cells (CAR-T cells) are T-cells which have been recombinantly modified by transduction with an expression vector encoding a CAR in substantial accordance with the teaching above. Prerequisite to transforming T-cells with an expression vector encoding the anti-targeting antigen CAR is to obtain a plurality of T-cells. T-cells useful in the preparation of CAR-T cells contemplated herein include naïve T-cells, central memory T-cells, effector memory T-cells or combination thereof.

In one embodiment, the CAR-T cell is prepared from a subject's own (autologous) T-cells by any of a variety of T-cell lines available in the art (e.g., Snook and Waldman (2013) Discovery Medicine 15(81):120-25). T-cells for transformation are typically obtained from the mammalian subject to be treated. T-cells can be obtained from a number of sources of the mammalian subject, including peripheral blood mononuclear cells, bone marrow, lymph node tissue, cord blood, thymus tissue, spleen tissue, and tumors. In one embodiment, T-cells are obtained by apheresis procedures such as leukapheresis. Leukapheresis is a process well known to those of skill in the art and may be achieved through the use of commercially available equipment including but not limited to the Haemonetics® Cell Saver® 5+, (commercially available from Haemonetics Corporation, 400 Wood Road, Braintree MA 02184) or COBE® 2991 cell processor (commercially available from TerumoBCT, Inc. 10811 West Collins Avenue, Lakewood CO 80215) in substantial accordance with the instructions provided by the manufacturer. In an alternative embodiment, the CAR-T cells may be allogenic (see, e.g. Gouble, et al., (2014) In vivo proof of concept of activity and safety of UCART19, an allogeneic “off-the-shelf” adoptive T-cell immunotherapy against CD19+ B-cell leukemias; Blood 124:4689.

In embodiment, T-cells are isolated from peripheral blood and particular T-cells (such as CD3⁺, CD28⁺, CD4⁺, CD8⁺, CD45RA⁺, and CD45RO⁺ T cells) can be isolated by selection techniques well known in the art such is incubation with anti-CD3/anti-CD28 conjugated beads. From the population of isolated T-cells, a subset of T-cells enriched for particular markers may be obtained. Typically, subsets of T-cells are isolated based on the expression one or more cell surface markers on the T-cells including but not limited to CD3+, CD4+, CD8+, CD25+, or CD62L+ T-cells. The preparation of a subset of T-cells enriched for one or more particular markers may be achieved by techniques well known in the art using commercially available instruments including but not limited to the CliniMACS® Plus and Prodigy (commercially available from Miltenyi Biotec Inc., 2303 Lindbergh Street, Auburn, CA 95602) in substantial accordance with the manufacturer's instructions. In one embodiment, a population enriched for CD3+ CAR-T cells is used for further processing. However, other subsets of T-cells such as naïve T-cells, central memory, or memory stem cells may also be used.

The processed T-cells prepared in substantial accordance with the above procedures may be used in further processing or cryopreserved.

### N. Transformation of T-Cells with CAR Expression Vector

Transduction of T-cells with the CAR expression vector may be accomplished using techniques well known in the art including but not limited co-incubation with host T-cells with viral vectors, electroporation, and/or chemically enhanced delivery. See, e.g., Naldini, et al. (1996) In Vivo Gene Delivery and Stable Transduction of Nondividing Cells by a Lentiviral Vector, Science 272: 263-267; Naldini, et al. (1996) Efficient transfer, integration, and sustained long-term expression of the transgene in adult rat brains injected with a lentiviral vector, Proc. Natl. Acad. Sci. USA Vol. 93, pp. 11382-11388; Dull, et al. (1998) A Third-Generation Lentivirus Vector with a Conditional Packaging System, J. Virology 72(11):8463-8471; Milone, et al. (2009) Chimeric Receptors Containing CD137 Signal Transduction Domains Mediate Enhanced Survival of T Cells and Increased Antileukemic Efficacy In Vivo, Molecular Therapy 17(8):1453-1464; Morgan and Boyerinas (2106) Genetic Modification of T Cells Biomedicines 4:9.

### O. Expansion of CAR-T Cells

Following transformation, T-cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005. Generally, the T-cells of the invention are expanded by culturing the cells in contact with a surface providing an agent that stimulates a CD3 TCR complex associated signal (e.g., an anti-CD3 antibody) and an agent that stimulates a co-stimulatory molecule on the surface of the T-cells (e.g an anti-CD28 antibody). Conditions appropriate for T-cell culture are well known in the art Lin, et al. (2009) Cytotherapy 11(7):912-922 (Optimization and validation of a robust human T-cell culture method for monitoring phenotypic and polyfunctional antigen-specific CD4 and CD8 T-cell responses); Smith, et al. (2015) Clinical & Translational Immunology 4:e31 published online 16 Jan. 2015 (“Ex vivo expansion of human T-cells for adoptive immunotherapy using the novel Xeno-free CTS Immune Cell Serum Replacement”). The target cells are maintained under conditions necessary to support growth, for example, an appropriate temperature (e.g., 37 °C) and atmosphere (e.g., air plus 5% CO₂). Ex vivo a T-cell activation may be achieved by procedures well established in the art including cel-based T-cell activation, antibody-based activation or activation using a variety of bead-based activation reagents. Cell-based T-cell activation may be achieved by exposure of the T-cells to antigen presenting cells, such as dendritic cells or artificial antigen presenting cells such as irradiated K562 cells. Antibody based activation of T-cell surface CD3 molecules with soluble anti-CD3 monoclonal antibodies also supports T-cell activation in the presence of IL-2 Alternatively bead-based T-cell activation, which has gained acceptance in the art for the preparation of CAR-T cells for clinical use. Bead-based activation of T-cells may be achieved using a wide variety of commercially available T-cell activation reagents including but not limited to the Invitrogen® CTS Dynabeads® CD3/28 (commercially available from Life Technologies, Inc. Carlsbad CA) or Miltenyi MACS® GMP ExpAct Treg beads or Miltenyi MACS GMP TransAct™ CD3/28 beads (commercially available from Miltenyi Biotec, Inc.). Several systems are available for the laboratory or commercial scale expansion of CAR-T cells including the GE WAVE bioreactor system, G-Rex bioreactors, the Miltenyi CliniMACS Prodigy system and recursive AAPC stimulation.

### P. Media

The present invention further provides media for the culture of CAR-T cells supplemented with an IL-10 agent. In one embodiment, the media of the present invention is a complete media is supplemented with IL-10 agent to achieve a concentration of the IL-10 agent at least 0.1 ng/ml, at least 0.2 ng/ml, at least 0.5 ng/ml, at least 1 ng/ml, at least 2 ng/ml, at least 3 ng/ml, at least 4 ng/ml, at least 5 ng/ml, at least 10 ng/ml, at least 50 ng/ml, at least 100 ng/ml, at least 200 ng/ml, at least 400 ng/ml, at least 500 ng/ml, at least 1000 ng/ml, at least 1500 ng/ml.

The level of IL-10 in the media should be maintained at level below the level at which the IL-10 is toxic to T-cells, optionally less than 50% of the toxic IL-10 agent concentration, optionally less than 30% of the toxic IL-10 agent concentration, optionally less than 20% of the toxic IL-10 agent concentration, or optionally less than 10% of the toxic IL-10 agent concentration.

Media useful for the culture and propagation of T-cells is well known in art. In the general practice of the technique of the culture of T-cells complete media. The typical complete media used for culture of leukocytes such as T-cells is RPMI media as described in Moore, G. E., et al. (1967) J.A.M.A., 199:519 and variants thereof as described in Moore, G. E. and Woods, L. K., “Culture media for human cells RPMI 1603, RPMI 1634, RPMI 1640 and GEM 1717.” Tissue Culture Association Manual, v. 3, 503-508 (1976). An exemplary formulation of RPMI media is the RPMI 1640 media obtainable from ThermoFisher Scientific (Carlsbad, CA) as catalog number 11875 having the following formulation in aqueous solution:

TABLE 2
RPMI 1640

| Component | Concentration (mg/L) |
|---|---|
| Glycine | 10.0 |
| L-Arginine | 200.0 |
| L-Asparagine | 50.0 |
| L-Aspartic acid | 20.0 |
| L-Cystine 2HCl | 65.0 |
| L-Glutamic Acid | 20.0 |
| L-Glutamine | 300.0 |
| L-Histidine | 15.0 |
| L-Hydroxyproline | 20.0 |
| L-Isoleucine | 50.0 |
| L-Leucine | 50.0 |
| L-Lysine hydrochloride | 40.0 |
| L-Methionine | 15.0 |
| L-Phenylalanine | 15.0 |
| L-Proline | 20.0 |
| L-Serine | 30.0 |
| L-Threonine | 20.0 |
| L-Tryptophan | 5.0 |
| L-Tyrosine disodium salt dihydrate | 29.0 |
| L-Valine | 20.0 |
| Biotin | 0.2 |
| Choline chloride | 3.0 |
| D-Calcium pantothenate | 0.25 |
| Folic Acid | 1.0 |
| Niacinamide | 1.0 |
| Para-Aminobenzoic Acid | 1.0 |
| Pyridoxine hydrochloride | 1.0 |
| Riboflavin | 0.2 |
| Thiamine hydrochloride | 1.0 |
| Vitamin B12 | 0.005 |
| i-Inositol | 35.0 |
| Calcium nitrate (Ca(NO3)2 4H2O) | 100.0 |
| Magnesium Sulfate (MgSO4) (anhyd.) | 48.84 |
| Potassium Chloride (KCl) | 400.0 |
| Sodium Bicarbonate (NaHCO3) | 2000.0 |
| Sodium Chloride (NaCl) | 6000.0 |
| Sodium Phosphate dibasic (Na2HPO4) | 800.0 |
| anhydrous | 2000.0 |
| D-Glucose (Dextrose) | 1.0 |
| Glutathione (reduced) | 5.0 |
| Phenol Red | |

### Q. Therapeutic and Prophylactic Uses

The present disclosure contemplates the use of the IL-10 agents described herein (e.g., PEG-IL-10) to enhance the therapeutic effect of CAR-T cell therapy. More specifically, IL-10 agents are used in methods directed to the modulation of a T-cell-mediated immune response to a target cell population in a subject, comprising introducing to the subject a therapeutically effective plurality of cells genetically modified to express a chimeric antigen receptor, wherein the chimeric antigen receptor comprises at least one antigen-specific targeting region capable of binding to the target cell population in combination with an IL-10 agent to enhance the cytoxic effect of the CAR-T cell therapy.

### R. Neoplasms Amenable to Treatment

The compositions and methods of the present invention are useful in the treatment of neoplasms, including benign and malignant neoplasms, and neoplastic disease. Examples benign neoplasms amenable to treatment using the compositions and methods of the present invention include but are not limited to adenomas, fibromas, hemangiomas, and lipomas. Examples of pre-malignant neoplasms amenable to treatment using the compositions and methods of the present invention include but are not limited to hyperplasia, atypia, metaplasia, and dysplasia. Examples of malignant neoplasms amenable to treatment using the compositions and methods of the present invention include but are not limited to carcinomas (cancers arising from epithelial tissues such as the skin or tissues that line internal organs), leukemias, lymphomas, and sarcomas typically derived from bone fat, muscle, blood vessels or connective tissues). Also included in the term neoplasms are viral induced neoplasms such as warts and EBV induced disease (i.e., infectious mononucleosis), scar formation, hyperproliferative vascular disease including intimal smooth muscle cell hyperplasia, restenosis, and vascular occlusion and the like.

The term “neoplastic disease” includes cancers characterized by solid tumors and non-solid tumors including but not limited to breast cancers; sarcomas (including but not limited to osteosarcomas and angiosarcomas), and fibrosarcomas), leukemias, lymphomas, genitourinary cancers (including but not limited to ovarian, urethral, bladder, and prostate cancers); gastrointestinal cancers (including but not limited to colon esophageal and stomach cancers); lung cancers; myelomas; pancreatic cancers; liver cancers; kidney cancers; endocrine cancers; skin cancers; and brain or central and peripheral nervous (CNS) system tumors, malignant or benign, including gliomas and neuroblastomas, astrocytomas, myelodysplastic disorders; cervical carcinoma-in-situ; intestinal polyposes; oral leukoplakias; histiocytoses, hyperprofroliferative scars including keloid scars, hemangiomas; hyperproliferative arterial stenosis, psoriasis, inflammatory arthritis; hyperkeratoses and papulosquamous eruptions including arthritis. In some embodiments, the ARD of the CAR is designed to interact with cell surface markers associated with non-cancer inflammatory and hyperproliferative conditions including not limited to CAR-T cell compositions, and associated methods of use of, including anti-AP CAR-T cells for the treatment of, for example, Alzheimers disease, anti-TNF CAR-T cells for the treatment of, for example, the treatment of arthritis, anti-IL17RA CAR-T cells for the treatment of, for example, placque psoriasis, anti-PSMA CAR-T cells for the treatment of, for example, prostate cancer and benign prostatic hyperplasia, anti-IL4RA CAR-T cells for the treatment of, for example, dermatitis, anti-PCSK9 CAR-T cells of, for example, the treatment of hypercholesterolemia, anti-VEGFR1 CAR-T cells for the treatment of, for example, age related macular degeneration, anti-VEGFR2 CAR-T cells for the treatment of, for example, age related macular degeneration, anti-IL-6R CAR-T cells for the treatment of, for example, rhumataoid arthritis, anti-IL-23 CAR-T cells for the treatment of, for example, psoriasis, arthritis, and crohns disease, and anti-CD4 CAR-T cells for the treatment of, for example, HIV infection.

The term “neoplastic diseases” includes myeloid neoplasms and lymphoid neoplasms. Each category contains different types of hematopoietic cancer with defining morphology, pathobiology, treatment, and/or prognostic features. Correct classification, along with identification of additional factors that may influence prognosis or response to chemotherapy, is essential to allow optimal treatment. Myeloid neoplasms include, but are not limited to, myeloproliferative neoplasms, myeloid and lymphoid disorders with eosinophilia, myeloproliferative/myelodysplastic neoplasms, myelodysplastic syndromes, acute myeloid leukemia and related precursor neoplasms, and acute leukemia of ambiguous lineage. Lymphoid neoplasms include, but are not limited to, precursor lymphoid neoplasms, mature B-cell neoplasms, mature T-cell neoplasms, Hodgkin's Lymphoma, and immunodeficiency-associated lymphoproliferative disorders. Other cancers of the hematopoietic system include, but are not limited to, histiocytic and dendritic cell neoplasms.

### S. Assessing Anti-Tumor Efficacy and Clinical Response

The determination of clinical efficacy in the treatment of cancer is generally associated with the achievement of one or more art recognized parameters such as reduction in lesions particularly reduction of metastatic lesion, reduction in metastatsis, reduction in tumor volume, improvement in ECOG score, and the like. Determining response to treatment can be assessed through the measurement of biomarker that can provide reproducible information useful in any aspect of IL-10 or immune pathway modulation, including the existence and extent of a subject's response to such therapy and the existence and extent of untoward effects caused by such therapy. By way of example, but not limitation, biomarkers include enhancement of IFNγ, and upregulation of granzyme A, granzyme B, and perforin; increase in CD8+ T-cell number and function; enhancement of IFNγ, an increase in ICOS expression on CD8+ T-cells, enhancement of IL-10 expressing T_{Reg} cells. Expression of the effector molecules IP-10 (Inducible Protein 10) and MIG (Monokine Induced by IFNγ) are known to be increased in certain IL-10-expressing tumors by either LPS or IFNγ; these effector molecules can also be leveraged as potential serum biomarkers that may be enhanced by the combinatorial therapies described herein. The response to treatment may be characterized by improvements in conventional measures of clinical efficacy may be employed such as Complete Response (CR), Partial Response (PR), Stable Disease (SD) and with respect to target lesions, Complete Response (CR),” Incomplete Response/Stable Disease (SD) as defined by RECIST as well as immune-related Complete Response (irCR), immune-related Partial Response (irPR), and immune-related Stable Disease (irSD) as defined Immune-Related Response Criteria (irRC) are considered by those of skill in the art as evidencing efficacy in the treatment of neoplastic disease in mammalian (e.g. human) subjects.

Further embodiments comprise a method or model for determining the optimum amount of an agent(s) in a combination. An optimum amount can be, for example, an amount that achieves an optimal effect in a subject or subject population, or an amount that achieves a therapeutic effect while minimizing or eliminating the adverse effects associated with one or more of the agents. In some embodiments, the elements of the combination of IL-10 and CAR-T cells itself is known to be, or has been determined to be, effective in treating or preventing a disease, disorder or condition described herein (e.g., a cancerous condition) in a subject (e.g., a human) or a subject population, and an amount of one agent is titrated while the amount of the other agent(s) is held constant. By manipulating the amounts of the agent(s) in this manner, a clinician is able to determine the ratio of agents most effective for, for example, treating a particular disease, disorder or condition, or eliminating the adverse effects or reducing the adverse effects such that are acceptable under the circumstances.

In particular embodiments, a therapeutically effective amount of the IL-10 agent (e.g., subcutaneously) and therapeutically effective plurality of CAR-T cells (e.g. intravenously) are administered parenterally to the subject. In other embodiments, a therapeutically effective plurality of cells genetically modified to express a chimeric antigen receptor and an IL-10 agent is introduced into the subject by intravenous infusion. In other embodiments, a therapeutically effective plurality of cells genetically modified to express a chimeric antigen receptor and an IL-10 agent is introduced into the subject by intratumoral injection. In other embodiments, a therapeutically effective plurality of cells genetically modified to express a chimeric antigen receptor and an IL-10 agent is introduced into the subject by loco-regional infusion. In still further embodiments, a therapeutically effective amount of the IL-10 agent sufficient to prevent or limit the activation-induced cell death is introduced into the subject by means of cells genetically modified to express the IL-10 agent, whereby the expression construct is present in different cells than those that express a CAR.

In those embodiments where the CAR-T cell also expresses the IL-10 agent, due to its the direct and local effect the amount of the IL-10 agent necessary to achieve a therapeutically effective amount may be significantly lower than that required to achieve a therapeutic effect through systemic administration of the IL-10 agent. As described herein, the levels of expression of IL-10 may be under the control of a regulatable promoter which facilitates modulation of the expression level of IL-10 in situ.

### T. Administration/Dosing

In general, dosing parameters of therapeutic agents dictate that the dosage amount be less than an amount that could be irreversibly toxic to the subject (i.e., the maximum tolerated dose, “MTD”) and not less than an amount required to produce a measurable effect on the subject. Such amounts are determined by, for example, the pharmacokinetic and pharmacodynamic parameters associated with ADME, taking into consideration the route of administration and other factors.

An “effective dose (ED)” is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount can be more than the calculated ED50, in other situations the effective amount can be less than the calculated ED50, and in still other situations the effective amount can be the same as the calculated ED50.

The therapeutic agents (e.g. IL-10 agents and CAR-T cells) of the present disclosure can be administered to a subject in an amount that is dependent upon, for example, the goal of the administration (e.g., the degree of resolution desired); the age, weight, sex, and health and physical condition of the subject the formulation being administered; and the route of administration. Therapeutically effective amounts and dosage regimens can be determined from, for example, safety and dose-escalation trials, in vivo studies (e.g., animal models), and other methods known to the skilled artisan.

### 1. Administration/Dosing of IL-10 Agents

In one embodiment, treatment with the IL-10 agent and the other agent(s) is maintained over a period of time. In another embodiment, treatment with the at least one other agent(s) is reduced or discontinued (e.g., when the subject is stable), while treatment with an IL-10 agent of the present disclosure (e.g., PEG-IL-10) is maintained at a constant dosing regimen. In a further embodiment, treatment with the other agent(s) is reduced or discontinued (e.g., when the subject is stable), while treatment with an IL-10 agent of the present disclosure is reduced (e.g., lower dose, less frequent dosing or shorter treatment regimen). In yet another embodiment, treatment with the other agent(s) is reduced or discontinued (e.g., when the subject is stable), and treatment with the IL-10 agent of the present disclosure is increased (e.g., higher dose, more frequent dosing or longer treatment regimen). In yet another embodiment, treatment with the other agent(s) is maintained and treatment with the IL-10 agent of the present disclosure is reduced or discontinued (e.g., lower dose, less frequent dosing or shorter treatment regimen). In yet another embodiment, treatment with the other agent(s) and treatment with an IL-10 agent of the present disclosure (e.g., PEG-IL-10) are reduced or discontinued (e.g., lower dose, less frequent dosing

The blood plasma levels of IL-10 in the methods described herein can be characterized in several manners, including: (1) a mean IL-10 serum trough concentration above some specified level or in a range of levels; (2) a mean IL-10 serum trough concentration above some specified level for some amount of time; (3) a steady state IL-10 serum concentration level above or below some specified level or in a range of levels; or (4) a Cₘₐₓ of the concentration profile above or below some specified level or in some range of levels. As set forth herein, mean serum trough IL-10 concentrations have been found to be of particular import for efficacy in certain indications.

In some embodiments, the IL-10 serum trough concentration is maintained over a period of a time at a level of greater than about 0.1 ng/mL, greater than about 0.2 ng/mL, greater than about 0.3 ng/mL, greater than about 0.4 ng/mL, greater than about 0.5 ng/mL, greater than about 0.6 ng/mL, greater than about 0.7 ng/mL, greater than about 0.8 ng/mL, greater than about 0.9 ng/mL, greater than about 1.0 ng/mL, greater than about 1.5 ng/mL, greater than about 2.0 ng/mL, greater than about 2.5 ng/mL, greater than about 3.0 ng/mL, greater than about 3.5 ng/mL, greater than about 4.0 ng/mL, greater than about 4.5 ng/mL, greater than about 5.0 ng/mL, greater than about 5.5 ng/mL, greater than about 6.0 ng/mL, greater than about 6.5 ng/mL, greater than about 7.0 ng/mL, greater than about 7.5 ng/mL, greater than about 8.0 ng/mL, greater than about 8.5 ng/mL, greater than about 9.0 ng/mL, greater than about 9.5 ng/mL, or greater than about 10.0 ng/mL.

In particular embodiments of the present disclosure, a mean IL-10 serum trough concentration is in the range of from 0.1 ng/mL to 10.0 ng/mL. In still other embodiments, the mean IL-10 serum trough concentration is in the range of from 1.0 ng/mL to 1 ng/mL. By way of example, the mean serum IL-10 concentration in an embodiment can be in the range of from 0.5 ng/mL to 5 ng/mL. By way of further examples, particular embodiments of the present disclosure comprise a mean IL-10 serum trough concentration in a range of from about 0.5 ng/mL to about 10.5 ng/mL, from about 1.0 ng/mL to about 10.0 ng/mL, from about 1.0 ng/mL to about 9.0 ng/mL, from about 1.0 ng/mL to about 8.0 ng/mL, from about 1.0 ng/mL to about 7.0 ng/mL, from about 1.5 ng/mL to about 10.0 ng/mL, from about 1.5 ng/mL to about 9.0 ng/mL, from about 1.5 ng/mL to about 8.0 ng/mL, from about 1.5 ng/mL to about 7.0 ng/mL, from about 2.0 ng/mL to about 10.0 ng/mL, from about 2.0 ng/mL to about 9.0 ng/mL, from about 2.0 ng/mL to about 8.0 ng/mL, and from about 2.0 ng/mL to about 7.0 ng/mL.

In particular embodiments, a mean IL-10 serum trough concentration of 1-2 ng/mL is maintained over the duration of treatment. The present disclosure also contemplates embodiments wherein the mean IL-10 serum peak concentration is less than or equal to about 10.0 ng/mL over the duration of treatment.

The present disclosure contemplates administration of any dose and dosing regimen that results in maintenance of any of the IL-10 serum trough concentrations set forth above over a period of time. By way of example, but not limitation, when the subject is a human, non-pegylated hIL-10 can be administered at a dose greater than 0.5 μg/kg/day, greater than 1.0 μg/kg/day, greater than 2.5 μg/kg/day, greater than 5 μg/kg/day, greater than 7.5 μg/kg, greater than 10.0 μg/kg, greater than 12.5 μg/kg, greater than 15 μg/kg/day, greater than 17.5 μg/kg/day, greater than 20 μg/kg/day, greater than 22.5 μg/kg/day, greater than 25 μg/kg/day, greater than 30 μg/kg/day, or greater than 35 μg/kg/day. In addition, by way of example, but not limitation, when the subject is a human, pegylated hIL-10 comprising a relatively small PEG (e.g., 5 kDa mono-di-PEG-hIL-10) can be administered at a dose greater than 0.5 μg/kg/day, greater than 0.75 μg/kg/day, greater than 1.0 μg/kg/day, greater than 1.25 μg/kg/day, greater than 1.5 μg/kg/day, greater than 1.75 μg/kg/day, greater than 2.0 μg/kg/day, greater than 2.25 μg/kg/day, greater than 2.5 μg/kg/day, greater than 2.75 μg/kg/day, greater than 3.0 μg/kg/day, greater than 3.25 μg/kg/day, greater than 3.5 μg/kg/day, greater than 3.75 μg/kg/day, greater than 4.0 μg/kg/day, greater than 4.25 μg/kg/day, greater than 4.5 μg/kg/day, greater than 4.75 μg/kg/day, or greater than 5.0 μg/kg/day.

In further embodiments, the aforementioned period of time over which the serum trough level of the IL-10 agent is maintained is at least 12 hours, at least 24 hours, at least 48 hours, at least 72 hours, at least 1 week, at least 2 weeks, at least 3 weeks, at least 1 month, at least 6 weeks, at least 2 months, at least 3 months, at least 6 months, at least 9 months, or greater than 12 months.

In particular embodiments of the present disclosure, the mean IL-10 serum trough concentration is maintained for at least 85% of the period of time, at least 90%, at least 96%, at least 98%, at least 99% or 100% of the period of time.

Although the preceding discussion regarding IL-10 serum concentrations, doses and treatment protocols that are necessary to achieve particular IL-10 serum concentrations, etc., pertains to monotherapy with an IL-10 agent (e.g., PEG-IL-10), the skilled artisan (e.g., a pharmacologist) is able to determine the optimum dosing regimen(s) when an IL-10 agent (e.g., PEG-IL-10) is administered in combination with one or more additional therapies.

### 2. Administration/Dosing of CAR-T Cell Agents

As previously discussed, the CAR-T agent is prepared using the patient's own T-cells as hosts for the recombinant vector encoding the CAR-T fusion protein. Consequently, the population of the cells to be administered is to the subject is necessarily variable. Additionally, since the CAR-T cell agent is variable, the response to such agents can vary and thus involves the ongoing monitoring and management of therapy related toxicities.

Based on animal models in mice, a dose of 5 million cells per animal per course of therapy demonstrates significant antitumor response. This dose, when scaled to a human is approximately equal to a dose of about 0.5×10¹⁰ viable CAR-T cells.

Typical ranges for the administration of CAR-T cells in the practice of the present invention range from about 1×10⁵ to 5×10⁸ viable CAR-T per kg of subject body weight per course of CAR-T cell therapy. Consequently, adjusted for body weight, typical ranges for the administration of viable T-cells in human subjects ranges from approximately 1×10⁶ to approximately 1×10¹³ viable CAR-T cells, alternatively from approximately 5×10⁶ to approximately 5×10¹², alternatively from approximately 1×10⁷ to approximately 1×10¹² alternatively from approximately 5×10⁷ to approximately 1×10¹² alternatively from approximately 1×10⁸ to approximately 1×10¹² alternatively from approximately 5×10⁸ to approximately 1×10¹² alternatively from approximately 1×10⁹ to approximately 1×10¹² for a course of therapy. In one embodiment, the dose of the CAR-T cells is in the range of 2.5-5×10⁹ viable CAR-T cells per course of therapy. The average number of T cells in a healthy adult is estimated to be approximately 1×10¹² cells, the dose ranges are less than approximately 1% of the total body mass of T cells. In some embodiments, the CAR-T cell therapy is Kymriah which is dosed in a single administration to patients ≤50 kg of 0.2 to 5.0×10⁶ CAR-positive viable T cells per kg body weight and to patients >50 kg, 0.1 to 2.5×10⁸ CAR-positive viable T cells.

A course of therapy with CAR-T cell agents may be a single dose or in multiple doses over a period of time. In some embodiments, the CAR-T cells are administered in a single dose. In some embodiments, the CAR-T cells are administered in two or more split doses administered over a period of 1, 2, 3, 4, 5, 6, 7, 8, 9, 10, 11, 12, 13, 14, 21, 28, 30, 60, 90 or 120 days. The amount of cells administered in such split dosing protocols may be the same in each administration or may provide different levels. For example, a course of therapy provide in a multiday three-dose split dosing protocol may provide for the administration of 10% on day 1, 30% on day 2 and 60% on day 3; alternatively 10% on day 1, 40% on day 2 and 50% on day 3; alternatively 25% on day 1, 25% on day 2 and 50% on day 3; alternatively 50% on day 1, 50% on day 14; alternatively 50% on day 1, 50% on day 7; alternatively 50% on day 1, 50% on day 30; alternatively 25% on day 1, 25% on day 14 and 50% on day 30; alternatively 50% on day 1, 25% on day 14 and 25% on day 30; alternatively 60% on day 1, 30% on day 14 and 10% on day 30; or, alternatively 50% on day 1, 25% on day 30 and 25% on day 60.

As previously discussed, the CAR-T agent may be prepared using the patient's own T-cells as hosts for the recombinant vector encoding the CAR-T fusion protein. Consequently, the population of the cells to be administered is to the subject is necessarily highly variable. Consequently, the dosages associated with the administration of CAR-T cell therapies is also variable and is frequently a function of management of toxicities. One form of toxicity associated with allogeneic or autologous T cell infusions in excessive immune response (including cytokine release syndrome) which is managed with a course of pharmacologic immunosuppression or B cell depletion. Examples of such immunosuppressive regimens including systemic corticolsteroids (e.g., methylprednisolone). Therapies for B cell depletion include intravenous immunoglobulin (IVIG) by established clinical dosing guidelines to restore normal levels of serum immunoglobulin levels.

In some embodiments, prior to administration of the CAR-T cell therapy of the present invention, the subject may optionally be subjected to a lymphodepleting regimen. One example of a such lymphodepleting regimen consists of the administration to the subject of fludarabine (30 mg/m² intravenous [IV] daily for 4 days) and cyclophosphamide (500 mg/m² IV daily for 2 days starting with the first dose of fludarabine). Such lymphodepletion has been associated with improved response in CAR-T cell therapies.

As noted herein, the administration of CAR-T cells in combination with IL-10 agents (and optionally additionally immunomodulatory and therapeutic agents) enhances the cytotoxic and immunomodulatory properties of CAR-T cells. Consequently, the levels of CAR-T cells conventionally employed in the treatment of a given disease, disorder or condition is may be reduced when combined with IL-10 agents to achieve a reduction in side effects potentially identified with CAR-T cell therapy. As such the present invention contemplates a method of reducing side effects associated with CAR-T cell therapy by administration of a CAR-T cell agent in combination with an IL-10 agent. Examples of side effects that may be mitigated by employing the compositions and methods of the present invention include but are not limited to cytokine release syndrome, off-target reactivity, immune suppression, and inflammation.

U. Combination with Additional Chemo-Therapeutic Agents:

In conjunction with the CAR-T cell and IL-10 agent combination therapy described herein, the present disclosure contemplates the addition of one or more active agents (“supplementary agents”) to the CAR-T cell and IL-10 agent combination therapy. Such further combinations are referred to as “supplementary combinations”, “supplementary combination therapy”, and agents that are added to the CAR-T cell and IL-10 agent combination therapy are referred to as “supplementary agents.”

As used herein, “supplementary combinations” is meant to include those combinations that can be administered or introduced separately, for example, formulated separately for separate administration (e.g., as may be provided in a kit), and therapies that can be administered or introduced together. In certain embodiments, the CAR-T cell and IL-10 agent combination therapy and the supplementary agent(s) are administered or applied sequentially, e.g., where one agent is administered prior to one or more other agents. In other embodiments, the CAR-T cell/IL-10 agent combination therapy and the supplementary agent(s) are administered simultaneously, e.g., where two or more agents are administered at or about the same time; the two or more agents may be present in two or more separate formulations or combined into a single formulation (i.e., a co-formulation). Regardless of whether the agents are administered sequentially or simultaneously, they are considered to be administered in combination for purposes of the present disclosure.

In one embodiment, the supplementary agent is a chemotherapeutic agent. The term “chemotherapeutic agents” includes but is not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate, folinic acid; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab-paclitaxel and doxetaxel; chlorambucil; gemcitabine; 6-thioguanine; mercaptopurine; methotrexate; platinum and platinum coordination complexes such as cisplatin, oxaplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; and pharmaceutically acceptable salts, acids or derivatives of any of the above. The term “chemotherapeutic agents” also includes anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above. In some embodiments, the supplementary agent may be one or more chemical or biological agents identified in the art as useful in the treatment of neoplastic disease, including, but not limited to, a cytokines or cytokine antagonists such as IL-12, INFα, or anti-epidermal growth factor receptor, radiotherapy, irinotecan; tetrahydrofolate antimetabolites such as pemetrexed; antibodies against tumor antigens, a complex of a monoclonal antibody and toxin, a T-cell adjuvant, bone marrow transplant, or antigen presenting cells (e.g., dendritic cell therapy), anti-tumor vaccines, replication competent viruses, signal transduction inhibitors (e.g., Gleevec® or Herceptin®) or an immunomodulator to achieve additive or synergistic suppression of tumor growth, non-steroidal anti-inflammatory drugs (NSAIDs), cyclooxygenase-2 (COX-2) inhibitors, steroids, TNF antagonists (e.g., Remicade® and Enbrel®), interferon-β1a (Avonex®), and interferon-β1b (Betaseron®) as well as combinations of one or more of the foregoing as practiced in known chemotherapeutic treatment regimens including but not limited to TAC, FOLFOX, TPC, FEC, ADE, FOLFOX-6, EPOCH, CHOP, CMF, CVP, BEP, OFF, FLOX, CVD, TC, FOLFIRI, PCV, FOLFOXIRI, ICE-V, XELOX, and others that are readily appreciated by the skilled clinician in the art.

In one embodiment, the supplementary agent is one or more non-pharmacological modalities (e.g., localized radiation therapy or total body radiation therapy). By way of example, the present disclosure contemplates treatment regimens wherein a radiation phase is preceded or followed by treatment with one or more additional therapies (e.g., CAR-T cell therapy and administration of an IL-10 agent) or agents as described herein. In some embodiments, the present disclosure further contemplates the use of CAR-T cell therapy and an IL-10 agent (e.g., PEG-IL-10) in combination with bone marrow transplantation, peripheral blood stem cell transplantation, or other types of transplantation therapy.

In one embodiment of the invention, prior to the administration of the CAR-T cells, the subject undergoes “chemopriming” to eliminate existing T-cells. In the typical practice chemopriming is achieved by the administration of one or more treatment modalities resulting in T-cell reduction or ablation including but not limited to cyclophosphamide chemotherapeutic regimens such as the combined administration of cyclophosphamide and fludacarbine, platinum based chemotherapeutic regimens, taxanes, temozolomide.

### V. Combination with Checkpoint Modulators

In another embodiment the “supplementary agent” is an immune checkpoint modulator for the treatment and/or prevention neoplastic disease in a subject as well as diseases, disorders or conditions associated with neoplastic disease. The term “immune checkpoint pathway” refers to biological response that is triggered by the binding of a first molecule (e.g. a protein such as PD1) that is expressed on an antigen presenting cell (APC) to a second molecule (e.g. a protein such as PDL1) that is expressed on an immune cell (e.g. a T-cell) which modulates the immune response, either through stimulation (e.g. upregulation of T-cell activity) or inhibition (e.g. downregulation of T-cell activity) of the immune response. The molecules that are involved in the formation of the binding pair that modulate the immune response are commonly referred to as “immune checkpoints.” The biological responses modulated by such immune checkpoint pathways are mediated by intracellular signaling pathways that lead to downstream immune effector pathways, such as cell activation, cytokine production, cell migration, cytotoxic factor secretion, and antibody production. Immune checkpoint pathways are commonly triggered by the binding of a first cell surface expressed molecule to a second cell surface molecule associated with the immune checkpoint pathway (e.g. binding of PD1 to PDL1, CTLA4 to CD28, etc.). The activation of immune checkpoint pathways can lead to stimulation or inhibition of the immune response.

An immune checkpoint whose activation results in inhibition or downregulation of the immune response is referred to herein as a “negative immune checkpoint pathway.” The inhibition of the immune response resulting from the activation of a negative immune checkpoint diminishes the ability of the host immune system to recognize foreign antigen such as a tumor-associated antigen. The term negative immune checkpoint pathway includes, but is not limited to, biological pathways modulated by the binding of PD1 to PDL1, PD1 to PDL2, and CTLA4 to CDCD80/86. Examples of such negative immune checkpoint antagonists include but are not limited to antagonists (e.g. antagonist antibodies) that bind T-cell inhibitory receptors including but not limited to PD1 (also referred to as CD279), TIM3 (T-cell membrane protein 3; also known as HAVcr2), BTLA (B and T lymphocyte attenuator; also known as CD272), the VISTA (B7-H5) receptor, LAG3 (lymphocyte activation gene 3; also known as CD233) and CTLA4 (cytotoxic T-lymphocyte associated antigen 4; also known as CD152).

In one embodiment, an immune checkpoint pathway the activation of which results in stimulation of the immune response is referred to herein as a “positive immune checkpoint pathway.” The term positive immune checkpoint pathway includes, but is not limited to, biological pathways modulated by the binding of ICOSL to ICOS(CD278), B7-H6 to NKp30, CD155 to CD96, OX40L to OX40, CD70 to CD27, CD40 to CD40L, and GITRL to GITR. Molecules which agonize positive immune checkpoints (such natural or synthetic ligands for a component of the binding pair that stimulates the immune response) are useful to upregulate the immune response. Examples of such positive immune checkpoint agonists include but are not limited to agonist antibodies that bind T-cell activating receptors such as ICOS (such as JTX-2011, Jounce Therapeutics), OX40 (such as MEDI6383, Medimmune), CD27 (such as varlilumab, Celldex Therapeutics), CD40 (such as dacetuzmumab CP-870,893, Roche, Chi Lob 7/4), HVEM, CD28, CD137 4-1BB, CD226, and GITR (such as MEDI1873, Medimmune; INCAGN1876, Agenus).

As used herein, the term “immune checkpoint pathway modulator” refers to a molecule that inhibits or stimulates the activity of an immune checkpoint pathway in a biological system including an immunocompetent mammal. An immune checkpoint pathway modulator may exert its effect by binding to an immune checkpoint protein (such as those immune checkpoint proteins expressed on the surface of an antigen presenting cell (APC) such as a cancer cell and/or immune T effector cell) or may exert its effect on upstream and/or downstream reactions in the immune checkpoint pathway. For example, an immune checkpoint pathway modulator may modulate the activity of SHP2, a tyrosine phosphatase that is involved in PD-1 and CTLA-4 signaling. The term “immune checkpoint pathway modulators” encompasses both immune checkpoint pathway modulator(s) capable of down-regulating at least partially the function of an inhibitory immune checkpoint (referred to herein as an “immune checkpoint pathway inhibitor” or “immune checkpoint pathway antagonist”) and immune checkpoint pathway modulator(s) capable of up-regulating at least partially the function of a stimulatory immune checkpoint (referred to herein as an “immune checkpoint pathway effector” or “immune checkpoint pathway agonist.”).

The immune response mediated by immune checkpoint pathways is not limited to T-cell mediated immune response. For example, the KIR receptors of NK cells modulate the immune response to tumor cells mediated by NK cells. Tumor cells express a molecule called HLA-C, which inhibits the KIR receptors of NK cells leading to a dimunition or the anti-tumor immune response. The administration of an agent that antagonizes the binding of HLA-C to the KIR receptor such an anti-KIR3 mab (e.g. lirilumab, BMS) inhibits the ability of HLA-C to bind the NK cell inhibitory receptor (KIR) thereby restoring the ability of NK cells to detect and attack cancer cells. Thus, the immune response mediated by the binding of HLA-C to the KIR receptor is an example a negative immune checkpoint pathway the inhibition of which results in the activation of a of non-T-cell mediated immune response.

In one embodiment, the immune checkpoint pathway modulator is a negative immune checkpoint pathway inhibitor/antagonist. In another embodiment, immune checkpoint pathway modulator employed in combination with the IL-10 agent is a positive immune checkpoint pathway agonist. In another embodiment, immune checkpoint pathway modulator employed in combination with the CAR-T cell and/or IL-10 agent is an immune checkpoint pathway antagonist.

As previously discussed, “negative immune checkpoint pathway inhibitor” refers to an immune checkpoint pathway modulator that interferes with the activation of a negative immune checkpoint pathway resulting in the upregulation or enhancement of the immune response. Exemplary negative immune checkpoint pathway inhibitors include but are not limited to programmed death-1 (PD1) pathway inhibitors, programed death ligand-1 (PDL1) pathway inhibitors, TIM3 pathway inhibitors and anti-cytotoxic T-lymphocyte antigen 4 (CTLA4) pathway inhibitors.

In one embodiment, the immune checkpoint pathway modulator is an antagonist of a negative immune checkpoint pathway that inhibits the binding of PD1 to PDL1 and/or PDL2 (“PD1 pathway inhibitor”). PD1 pathway inhibitors result in the stimulation of a range of favorable immune response such as reversal of T-cell exhaustion, restoration cytokine production, and expansion of antigen-dependent T-cells. PD1 pathway inhibitors have been recognized as effective variety of cancers receiving approval from the USFDA for the treatment of variety of cancers including melanoma, lung cancer, kidney cancer, Hodgkins lymphoma, head and neck cancer, bladder cancer and urothelial cancer.

The term PD1 pathway inhibitors includes monoclonal antibodies that interfere with the binding of PD1 to PDL1 and/or PDL2. Antibody PD1 pathway inhibitors are well known in the art. Examples of commercially available PD1 pathway inhibitors that monoclonal antibodies that interfere with the binding of PD1 to PDL1 and/or PDL2 include nivolumab (Opdivo®, BMS-936558, MDX1106, commercially available from BristolMyers Squibb, Princeton NJ), pembrolizumab (Keytruda®MK-3475, lambrolizumab, commercially available from Merck and Company, Kenilworth NJ), and atezolizumab (Tecentriq®, Genentech/Roche, South San Francisco CA). Additional PD1 pathway inhibitors antibodies are in clinical development including but not limited to durvalumab (MEDI4736, Medimmune/AstraZeneca), pidilizumab (CT-011, CureTech), PDR001 (Novartis), BMS-936559 (MDX1105, BristolMyers Squibb), and avelumab (MSB0010718C, Merck Serono/Pfizer) and SHR-1210 (Incyte). Additional antibody PD1 pathway inhibitors are described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, U.S. Pat. No. 7,943,743 (Medarex, Inc) issued May 17, 2011.

In one embodiment of the invention, the PD1 immune checkpoint pathway modulator is an antibody comprising the CDR sequences provided in Table 3 below:

TABLE 3
CDR Sequences

| Name | Amino Acid Sequence | SEQ ID NO |
|---|---|---|
| CDR-L1 | GGNSIGSYSVH | SEQ ID NO 50 |
| CDR-L2 | DDSDRPS | SEQ ID NO 51 |
| CDR-L3 | QVWDTSSYWV | SEQ ID NO 52 |
| CDR-H1 | GFTFSSYAMS | SEQ ID NO 53 |
| CDR-H2 | DISGGGGTTYYADSVKG | SEQ ID NO 54 |
| CDR-H3 | SGTVVTDFDY | SEQ ID NO 55 |

In one embodiment of the invention, the PD1 immune checkpoint pathway inhibitor is an antibody comprising the variable domain sequences (SEQ ID NO: 56 and SEQ ID NO: 57) provided in Table 4 below:

TABLE 4
Variable Domain Sequences

| Name | Amino Acid Sequence | SEQ ID NO: |
|---|---|---|
| VL-09 | SYVLTQPPSVSVAPGQTARVTCGGNSIGSYS | SEQ ID NO 56 |
| | VHWYQQKPGQAPVLVVYDDSDRPSGIPERFS | |
| | GSNSGNTAALTISRVEAGDEADYYCQVWDTS | |
| | SWVFGGGTKLTVL | |
| VH-09 | EVQLLESGGGLVQPGGSLRLSCPASGFTFSS | SEQ ID NO 57 |
| | YAMSWVRQAPGKGLGWVSDISGGGGTTYYAD | |
| | SVKGRFTISRDNSKNTLYLQMNSLRGEDTAV | |
| | YYCAKSGTVVTDFDWGQGTLVTVSS | |

In one embodiment of the invention, the PD1-antagonist antibody is AM0001: a monoclonal antibody with a lambda 2 light chain and an IgG4 with a serine to proline substitution at position 228 (S228P) to provide a “hinge-stabilized” heavy chain, characterized by VL and VH CDRs having amino acid sequences corresponding to SEQ ID NOS: 50-55 as set out in Table 3 above, a light chain variable region characterized by the sequence of SEQ ID NO: 56 and a heavy chain variable region characterized by the amino acid sequence of SEQ ID NO:57. The AM0001 antibody is characterized as having a binding affinity (K_{d}) for human and cynomologous monkey PD-1 of about 10 pM or less at 25 °C. The binding affinity of AM0001, measured by bio-layer interferometry (BLI), are shown in Table 5 below.

TABLE 5
PD-1 Binding Affinity AM0001

| Antibody | K_{D}(M) | Kₒₙ | K_{dis} | R² |
|---|---|---|---|---|
| AM0001 | <1.0E−12 | 6.655E+5 | <1.0E−7 | 0.9989 |

The full length amino acid sequences of the heavy chain and light chain of AM0001 are provided below.

AM0001 Mature Heavy Chain Protein Sequence
(Human IgG4 S228P Framework):
(SEQ ID NO: 60)
EVQLLESGGGLVQPGGSLRLSCPASGFTFSSYAMSWVRQAPGKGLGWVSD
ISGGGGTTYYADSVKGRFTISRDNSKNTLYLQMNSLRGEDTAVYYCAKSG
TVVTDFDYWGQGTLVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
AM0001 Mature Light Chain Protein Sequence
(Human Lambda-2 Framework):
(SEQ ID NO: 61)
SYVLTQPPSVSVAPGQTARVTCGGNSIGSYSVHWYQQKPGQAPVLVVYDD
SDRPSGIPERFSGSNSGNTAALTISRVEAGDEADYYCQVWDTSSYWVFGG
GTKLTVLGQPKAAPSVTLFPPSSEELQANKATLVCLISDFYPGAVTVAWK
ADSSPVKAGVETTTPSKQSNNKYAASSYLSLTPEQWKSHRSYSCQVTHEG
STVEKTVAPTECS

The PD-1 pathway inhibitor antibody may be produced by recombinant means. The present invention includes nucleic acid sequences encoding the amino acid sequences of SEQ ID NO. 50, SEQ ID NO. 51, SEQ ID NO. 52, SEQ ID NO. 53, SEQ ID NO. 54, SEQ ID NO. 5, SEQ ID NO. 56, SEQ ID NO. 57, SEQ ID NO. 60, and SEQ ID NO. 61. In one embodiment, the present disclosure provides nucleic acid sequences when the PD1-antagonist antibody is AM0001, the nucleic acid sequences encoding the heavy and light chains of AM0001 (SEQ ID NO. 60 and SEQ ID NO. 61) are as set out below as SEQ ID NO. 62, and SEQ ID NO. 63, respectively.

AM0001 Mature Heavy Chain DNA Sequence
(Human IgG4 S228P Framework):
(SEQ ID NO: 62)
GAGGTCCAGCTCCTGGAATCCGGGGGCGGTCTGGTCCAGCCGGGCGGCTC
GCTCCGCCTGTCCTGCCCGGCGAGCGGCTTCACCTTCTCCTCCTACGCCA
TGTCCTGGGTGAGGCAGGCCCCCGGCAAGGGCCTCGGCTGGGTCAGCGAC
ATCTCCGGCGGCGGCGGCACCACGTACTACGCGGACTCGGTGAAGGGCCG
GTTCACGATCTCCCGGGACAACTCCAAGAACACCCTGTACCTGCAGATGA
ACTCACTGCGGGGCGAGGACACGGCGGTGTATTACTGCGCCAAGTCCGGA
ACGGTTGTGACTGATTTCGACTACTGGGGCCAGGGCACCCTGGTGACCGT
GTCCAGCGCCTCCACCAAGGGCCCCAGCGTGTTCCCCCTGGCGCCGTGCT
CGCGGAGCACCAGCGAGTCCACCGCCGCGCTCGGTTGCCTCGTCAAGGAC
TACTTCCCCGAGCCGGTCACAGTGTCATGGAACTCCGGCGCGCTGACGAG
CGGCGTGCACACCTTCCCGGCCGTGCTCCAGTCCAGCGGCCTGTACAGCC
TCAGTAGCGTCGTGACGGTGCCCTCGTCGTCGCTGGGCACGAAGACCTAC
ACCTGCAACGTGGACCACAAGCCGTCCAACACCAAGGTCGATAAGCGAGT
GGAGAGCAAGTACGGCCCCCCGTGCCCCCCCTGCCCGGCCCCGGAGTTCC
TGGGTGGCCCCTCCGTGTTCCTCTTCCCCCCGAAGCCCAAAGACACCCTC
ATGATCAGCCGGACGCCGGAGGTCACGTGCGTCGTCGTGGACGTGAGCCA
GGAAGACCCGGAGGTCCAGTTCAACTGGTACGTGGACGGCGTCGAGGTGC
ATAACGCCAAGACCAAGCCTCGCGAGGAACAGTTCAACTCCACTTACCGC
GTCGTGTCCGTCCTCACCGTCCTGCACCAGGACTGGCTCAACGGGAAGGA
ATACAAGTGCAAGGTCTCGAACAAGGGCCTGCCGTCGTCCATCGAGAAGA
CCATCAGCAAGGCCAAGGGCCAGCCGCGGGAGCCCCAGGTCTACACCCTC
CCCCCCTCCCAGGAAGAGATGACGAAGAACCAGGTGAGCCTGACGTGCCT
CGTGAAGGGGTTCTACCCCTCCGACATCGCAGTCGAGTGGGAGAGCAACG
GCCAGCCGGAGAACAACTACAAGACGACCCCCCCGGTGCTGGACAGCGAC
GGGTCCTTCTTCCTCTACTCGCGTCTCACAGTCGACAAGTCGCGCTGGCA
GGAGGGCAACGTCTTCTCGTGCTCCGTGATGCACGAGGCCCTGCACAACC
ACTACACCCAGAAGTCGCTGTCCCTGTCCCTGGGCAAG
AM0001 Mature Light Chain Protein Sequence
(Human Lambda-2 Framework):
(SEQ ID NO: 63)
AGCTACGTGCTGACCCAGCCGCCCTCGGTGTCGGTCGCCCCGGGCCAGAC
GGCACGTGTGACCTGCGGCGGTAACAGCATCGGCTCCTACTCGGTCCACT
GGTATCAGCAGAAGCCGGGGCAGGCCCCGGTCCTGGTGGTCTACGACGAC
AGCGACCGCCCGTCCGGCATCCCCGAACGCTTCAGCGGCTCAAACAGCGG
GAACACCGCGGCCCTGACGATCTCGCGCGTCGAGGCGGGGGACGAAGCCG
ATTACTACTGCCAGGTCTGGGACACCTCGAGTTACTGGGTGTTCGGCGGG
GGCACGAAGCTGACCGTCCTCGGCCAGCCGAAGGCCGCCCCCTCAGTAAC
CCTGTTCCCCCCGTCCTCGGAGGAGTTGCAGGCGAACAAGGCGACGCTGG
TGTGCTTGATCTCGGACTTCTACCCCGGAGCGGTGACGGTCGCCTGGAAG
GCCGACTCCTCCCCGGTCAAGGCGGGCGTGGAGACGACCACCCCCTCCAA
GCAGAGCAACAACAAGTACGCCGCCTCGAGCTACCTCTCGCTGACACCCG
AGCAGTGGAAGTCCCACCGGTCCTACTCGTGCCAGGTAACCCACGAGGGC
TCCACCGTCGAGAAGACCGTGGCCCCCACCGAGTGCAGC

The term PD1 pathway inhibitors are not limited to antagonist antibodies. Non-antibody biologic PD1 pathway inhibitors are also under clinical development including AMP-224, a PD-L2 IgG2a fusion protein, and AMP-514, a PDL2 fusion protein, are under clinical development by Amplimmune and Glaxo SmithKline. Aptamer compounds are also described in the literature useful as PD1 pathway inhibitors (Wang, et al. Selection of PD1/PD-L1 X-Aptamers, Biochimie, in press; available online 11 Sep. 2017, at the internet address: https://doi.org/10.1016/j.biochi.2017.09.006.

The term PD1 pathway inhibitors includes peptidyl PD1 pathway inhibitors such as those described in Sasikumar, et al., U.S. Pat. No. 9,422,339 issued Aug. 23, 2016, and Sasilkumar, et al., U.S. Pat. No. 8,907,053 issued Dec. 9, 2014. CA-170 (AUPM-170, Aurigene/Curis) is reportedly an orally bioavailable small molecule targeting the immune checkpoints PDL1 and VISTA. Pottayil Sasikumar, et al. Oral immune checkpoint antagonists targeting PD-L1/VISTA or PD-L1/Tim3 for cancer therapy. [abstract]. In: Proceedings of the 107th Annual Meeting of the American Association for Cancer Research; 2016 Apr. 16-20; New Orleans, LA. Philadelphia (PA): AACR; Cancer Res 2016; 76(14 Suppl): Abstract No. 4861. CA-327 (AUPM-327, Aurigene/Curis) is reportedly an orally available, small molecule that inhibit the immune checkpoints, Programmed Death Ligand-1 (PDL1) and T-cell immunoglobulin and mucin domain containing protein-3 (TIM3).

The term PD1 pathway inhibitors includes small molecule PD1 pathway inhibitors. Examples of small molecule PD1 pathway inhibitors useful in the practice of the present invention are described in the art including Sasikumar, et al 1,2,4-oxadiazole and thiadiazole compounds as immunomodulators (PCT/IB2016/051266 filed Mar. 7, 2016, published as WO2016142833A1 Sep. 15, 2016) and Sasikumar, et al. 3-substituted-1,2,4-oxadiazole and thiadiazole compounds as immunomodulators (PCT application serial number PCT/IB2016/051343 filed Mar. 9, 2016 and published as WO2016142886A2), BMS-1166 and BMS-1001 (Skalniak, et al (2017) Oncotarget 8(42): 72167-72181) having the structures:

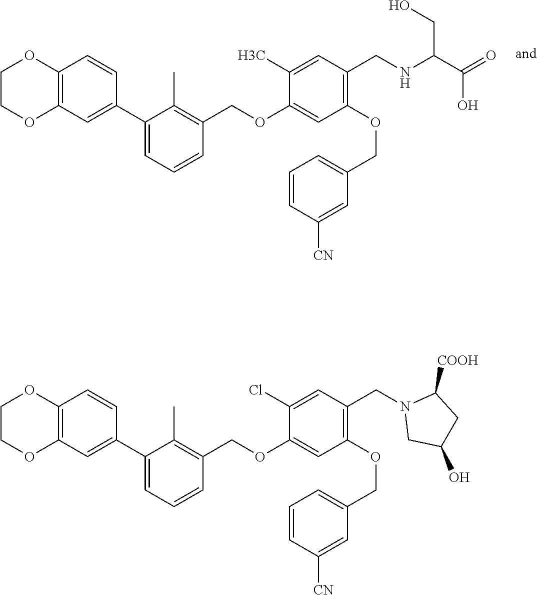 [SMILES: Cc1cc(CNC(CO)C(=O)O)c(OCc2cccc(C#N)c2)cc1OCc1cccc(-c2ccc3c(c2)OCCO3)c1C ; Cc1c(COc2cc(OCc3cccc(C#N)c3)c(CN3C[C@H](O)C[C@@H]3C(=O)O)cc2Cl)cccc1-c1ccc2c(c1)OCCO2]

Chupak L S amd Zheng X. Compounds useful as immunomodulators. Bristol-Myers Squibb Co. 2015 WO 2015/034820 A1, EP3041822 B1 granted Aug. 9, 2017; WO2015034820 A1; and Chupak, et al. Compounds useful as immunomodulators. Bristol-Myers Squibb Co. 2015 WO 2015/160641 A2. WO 2015/160641 A2, Chupak, et al. Compounds useful as immunomodulators. Bristol-Myers Squibb Co. Sharpe, et al Modulators of immunoinhibitory receptor pd-1, and methods of use thereof, WO 2011082400 A2 published Jul. 7, 2011; U.S. Pat. No. 7,488,802 (Wyeth) issued Feb. 10, 2009;

The CAR-T cell and/or IL-10 agent compositions and methods of the present disclosure are particularly suited for treatment of neoplastic conditions for which PD1 pathway inhibitors have demonstrated clinical effect in human beings either through FDA approval for treatment of the disease or the demonstration of clinical efficacy in clinical trials including but not limited to melanoma, non-small cell lung cancer, small cell lung cancer, head and neck cancer, renal cell cancer, bladder cancer, ovarian cancer, uterine endometrial cancer, uterine cervical cancer, uterine sarcoma, gastric cancer, esophageal cancer, DNA mismatch repair deficient colon cancer, DNA mismatch repair deficient endometrial cancer, hepatocellular carcinoma, breast cancer, Merkel cell carcinoma, thyroid cancer, Hodgkins lymphoma, follicular lymphoma, diffuse large B-cell lymphoma, mycosisfungoides, peripheral T-cell lymphoma.

Perhaps the most well studied immunotherapy with the greatest clinical experience has been obtained with the anti-PD1 monoclonal antibodies pembrolizumab (Keytruda®) and nivolumab. These products have demonstrated significant effectiveness and now currently enjoy multiple approvals for a wide variety of cancers. The clinical experience with these agents has demonstrated a series of parameters which point to a greatest chance of success. Anti-PD1 therapy has demonstrated highest levels of effectiveness in those tumors where there are high levels of expression of PDL1 Garon, et al. NEJM 2014, where the tumor has a tumor mutational burden (Rizvi et al., Science 2015; Carbone et al. NEJM 2017, where there are high levels of CD8+ T-cell in the tumor Tumeh, et al., Nature 2014, an immune activation signature associated with IFNγ Prat, et al., Cancer Res. 2017; Ayers et al JCI 2017, and the lack of metastatic disease particularly liver metastasis Tumeh et al. Cancer Imm. Res. 2017; Pillai, et al. ASCO 2017. These factors limit the effectiveness of PD1 therapy to a comparatively small range of tumors. A wide variety of tumors have low neo-antigen burden with rare neoantigen specific CD8+ T-cells, and tumors with high neo-antigen burden have been eventually escape ICIs. In other situations, there is an Immune Desert in the tumor microenvironment where T-cells have exhausted and apoptosed, the lack of T-cell expression leads low levels of granzyme and IFNγ expression in the tumor. IL-10 monotherapy addresses many of these parameters. IL-10 has been observed to increase activity of increase activity of intratumoral CD8+ T-cells, increase levels of granzymes, FasL and IFNγ. Mumm, et al., (2011) Cancer Cell; Emmerich et al., (2012) Cancer Research; Oft, et al. (2014) Cancer Immunology Research. Because of the established utility of IL-10 in addressing these hurdles (as presented on previous slide) we evaluated an IL-10 agent in combination with anti-PD1 Mab therapy.

In one embodiment, the immune checkpoint pathway modulator is an antagonist of a negative immune checkpoint pathway that inhibits the binding of CTLA4 to CD28 (“CTLA4 pathway inhibitor”). The immune checkpoint receptor CTLA4 belongs to the immunoglobulin superfamily of receptors, which also includes PD1; BTLA; lymphocyte attenuator; TIM3, and V-domain immunoglobulin suppressor of T-cell activation. CD80 (also known as B7.1) and CD86 (also known as B7.2) have been identified as the CTLA4 receptor ligands. CTLA4, the first immune checkpoint receptor to be clinically targeted, is expressed exclusively on T-cells, where it primarily regulates the amplitude of the early stages of T-cell activation. It has been shown to counteract the activity of the T-cell co-stimulatory receptor CD28.

Upon antigen recognition, CD28 signaling strongly amplifies T-cell receptor signaling to activate T-cells. [See, e.g., Riley et al., (2002) Proc. Natl Acad. Sci. USA 99:11790-95]. CTLA4 is transcriptionally induced following T-cell activation. Although CTLA4 is expressed by activated CD8+ effector T-cells, its primary physiological role is believed to be manifested through distinct effects on the two major subsets of CD4+ T-cells: i) down-modulation of helper T-cell activity, and ii) enhancement of regulatory T-cell immunosuppressive activity. Specifically, CTLA4 blockade results in immune response enhancement dependent on helper T-cells, while CTLA4 engagement of regulatory T-cells increases their suppressive function. [See, e.g., Fontenot et al., (2003) Nat. Immunol. Proc. 4:330-36]. Examples of CTLA4 pathway inhibitor are well known in the art (See, e.g., U.S. Pat. No. 6,682,736 (Abgenix) issued Jan. 27, 2004; U.S. Pat. No. 6,984,720 (Medarex, Inc.) issued May 29, 2007; U.S. Pat. No. 7,605,238 (Medarex, Inc.) issued Oct. 20, 2009).

Currently CTLA4 pathway inhibitor antibody treatment approaches are not without shortcomings. By way of example, treatment of metastatic melanomas with a humanized anti-CTLA4 antagonistic antibody has been reported to cause certain autoimmune toxicities (e.g., bowel inflammation and dermatitis), prompting the determination of a tolerated therapeutic window (Wu et al., (2012) Int. J. Biol. Sci. 8:1420-30). The enhanced therapeutic efficacy of the combination of an CTLA4 pathway inhibitor (e.g., an antibody such as ipilimumab) with IL-10 agent (e.g., PEG-IL-10) offers the potential of reducing dosages while maintaining therapeutic efficacy.

In one embodiment, the immune checkpoint pathway modulator is an antagonist of a negative immune checkpoint pathway that inhibits the binding of BTLA to HVEM (“BTLA pathway inhibitor”) BTLA is a co-inhibitory molecule structurally and functionally related to CTLA-4 and PD-1. Although BTLA is expressed on virus-specific human CD8+ T-cells, it is progressively downregulated after their differentiation from a naïve to effector phenotype (Paulos et al., (January 2010) J. Clin. Invest. 120(1):76-80). The herpes virus entry mediator (HVEM; also known as TNFRSF14), which is expressed on certain tumor cell types (e.g., melanoma) and tumor-associated endothelial cells, has been identified as the BTLA ligand. Because the interactions between BTLA and HVEM are complex, therapeutic inhibition strategies are less straightforward for BTLA than they are for other immune checkpoint pathway inhibitory receptors and ligands. [Pardoll, (April 2012) Nature Rev. Cancer 12:252-64]. A number of approaches targeting the BTLA/HVEM pathway using anti-BTLA antibodies and antagonistic HVEM-Ig have been evaluated, and such approaches have suggested promising utility in a number of diseases, disorders and conditions, including transplantation, infection, tumor, and autoimmune disease (Wu et al., (2012) Int. J. Biol. Sci. 8:1420-30).

In one embodiment, the immune checkpoint pathway modulator is an antagonist of a negative immune checkpoint pathway that inhibits the ability TIM3 to binding to TIM3-activating ligands (“TIM3 pathway inhibitor”). TIM3 inhibits T helper 1 (TH1) cell responses, and anti-TIM3 antibodies have been shown to enhance antitumor immunity. Galectin 9, a molecule involved in the modulation of the TIM3 pathway, is upregulated in various types of cancer, including breast cancer. TIM3 has been reported to be co-expressed with PD1 on tumor-specific CD8+ T-cells. When stimulated by the cancer-testes antigen NY-ESO-1, dual inhibition of both molecules significantly enhances the in vitro proliferation and cytokine production of human T-cells. Moreover, in animal models, coordinated blockage of PD1 and TIM3 was reported to enhance antitumor immune responses in circumstances in which only modest effects from blockade of each individual molecule were observed. [See, e.g., Pardoll, (April 2012) Nature Rev. Cancer 12:252-64; Zhu et al., (2005) Nature Immunol. 6:1245-52; Ngiow et al., (2011) Cancer Res. 71:3540-51)]. Examples of TIM3 pathway inhibitors are known in the art and with representative non-limiting examples described in United States Patent Publication No. PCT/US2016/021005 published Sep. 15, 2016; Lifke, et al. United States Patent Publication No. US 20160257749 A1 published Sep. 8, 2016 (F. Hoffman-LaRoche), Karunsky, U.S. Pat. No. 9,631,026 issued Apr. 27, 2017; Karunsky, Sabatos-Peyton, et al. U.S. Pat. No. 8,841,418 issued Sep. 23, 2014; U.S. Pat. No. 9,605,070; Takayanagi, et al U.S. Pat. No. 8,552,156 issued Oct. 8, 2013.

LAG3 has been shown to play a role in enhancing the function of Regulatory T (T_{Reg}) cells, and independently in inhibiting CD8+ effector T-cell functions. MHC class II molecules, the ligand for LAG3, are upregulated on some epithelial cancers (often in response to IFNγ), and are also expressed on tumor-infiltrating macrophages and dendritic cells. Though the role of the LAG3-MHC class II interaction has not been definitively elucidated, the interaction can be a key component in the role of LAG3 in enhancing T_{Reg} cell function.

LAG3 is one of several immune checkpoint receptors that are coordinately upregulated on both T_{Reg} cells and anergic T-cells. Simultaneous blockade of LAG3 and PD1 can cause enhanced reversal of the anergic state when compared to blockade of one receptor alone. Indeed, blockade of LAG3 and PD1 has been shown to synergistically reverse anergy among tumor-specific CD8+ T-cells and virus-specific CD8+ T-cells in the setting of chronic infection. IMP321 (ImmuFact) is being evaluated in melanoma, breast cancer, and renal cell carcinoma. [See generally Woo et al., (2012) Cancer Res 72:917-27; Goldberg et al., (2011) Curr. Top. Microbiol. Immunol. 344:269-78; Pardoll, (April 2012) Nature Rev. Cancer 12:252-64; Grosso et al., (2007) J. Clin. Invest. 117:3383-392].

A2aR inhibits T-cell responses by stimulating CD4+ T-cells towards developing into T_{Reg} cells. A2aR is particularly important in tumor immunity because the rate of cell death in tumors from cell turnover is high, and dying cells release adenosine, which is the ligand for A2aR. In addition, deletion of A2aR has been associated with enhanced and sometimes pathological inflammatory responses to infection. Inhibition of A2aR can be effected by antibodies that block adenosine binding or by adenosine analogs. Such agents can be useful in disorders such as cancer and Parkinson's disease. [See generally, Zarek et al., (2008) Blood 111:251-59; Waickman et al., (25 Nov. 2011) Cancer Immunol. Immunother. (doi: 10. 1007/s00262-011-1155-7)].

IDO (Indoleamine 2,3-dioxygenase) is an immune regulatory enzyme that is normally expressed in tumor cells and in activated immune cells. IDO down-regulates the immune response mediated through oxidation of tryptophan. This results in inhibition of T-cell activation and induction of T-cell apoptosis, creating an environment in which tumor-specific cytotoxic T lymphocytes are rendered functionally inactive or are no longer able to attack a subject's cancer cells. Indoximod (NewLink Genetics) is an IDO inhibitor being evaluated in metastatic breast cancer.

Production, purification, and fragmentation of polyclonal and monoclonal antibodies are described (e.g., Harlow and Lane (1999) Using Antibodies, Cold Spring Harbor Laboratory Press, Cold Spring Harbor, NY); standard techniques for characterizing ligand/receptor interactions are available (see, e.g., Coligan et al. (2001) Current Protocols in Immunology, Vol. 4, John Wiley, Inc., NY); methods for flow cytometry, including fluorescence-activated cell sorting (FACS), are available (see, e.g., Shapiro (2003) Practical Flow Cytometry, John Wiley and Sons, Hoboken, NJ); and fluorescent reagents suitable for modifying nucleic acids, including nucleic acid primers and probes, polypeptides, and antibodies, for use, for example, as diagnostic reagents, are available (Molecular Probes (2003) Catalogue, Molecular Probes, Inc., Eugene, OR.; Sigma-Aldrich (2003) Catalogue, St. Louis, MO.).

As previously described, the present invention provides for a method of treatment of neoplastic disease (e.g. cancer) in a mammalian subject by the administration of a CAR-T cell and/or IL-10 agent (e.g., PEG-IL-10) in combination with an agent(s) that modulate at least one immune checkpoint pathway including immune checkpoint pathway modulators that modulate two, three or more immune checkpoint pathways.

In one embodiment, multiple immune checkpoint pathways may be modulated by the administration of multi-functional molecules which are capable of acting as modulators of multiple immune checkpoint pathways. Examples of such multi-immune checkpoint pathway modulators include but are not limited to bi-specific or poly-specific antibodies. Examples of poly-specific antibodies capable of acting as modulators or multiple immune checkpoint pathways are known in the art. For example, United States Patent Publication No. 2013/0156774 describes bispecific and multispecific agents (e.g., antibodies), and methods of their use, for targeting cells that co-express PD1 and TIM3. Moreover, dual blockade of BTLA and PD1 has been shown to enhance antitumor immunity (Pardoll, (April 2012) Nature Rev. Cancer 12:252-64). The present disclosure contemplates the use of IL-10 agents in combination with immune checkpoint pathway modulators that target multiple immune checkpoint pathways, including but limited to bi-specific antibodies which bind to both PD1 and LAG3. Thus, antitumor immunity can be enhanced at multiple levels, and combinatorial strategies can be generated in view of various mechanistic considerations.

Other embodiments contemplate the administration of an IL-10 agent in combination with multiple checkpoint pathway modulators, and still further embodiments contemplate the administration of an IL-10 agent in combination with three or more immune checkpoint pathway modulators. Such combinations of CAR-T cell and/or IL-10 agents with multiple immune checkpoint pathway modulators can be advantageous in that immune checkpoint pathways may have distinct mechanisms of action, which provides the opportunity to attack the underlying disease, disorder or conditions from multiple distinct therapeutic angles. Representative combinations (some of which are in clinical trials as identified below) of immune checkpoint pathway modulators that may be combined with the administration of an IL-10 agent include but are not limited to:
(a) PD1/PDL1 pathway inhibitors (including but not limited to nivolumab, pembrolizumab, PDR001; MEDI4736, atezolizumab, and durvalumab) with LAG3 antagonist antibodies (e.g. BMS-986016, clinical trial identifier NCT01968109), CTLA4 antagonist antibodies (e.g. ipilumumab), B7-H3 antagonist antibodies (e.g. enoblituzumab, e.g. clinical trial identifier NCT01968109), KIR antagonist antibodies (e.g. lirilumab, e.g. clinical trial identifier NCT01714739);
(b) PD1/PDL1 pathway inhibitors (including but not limited to nivolumab, pembrolizumab, PDR001; MEDI4736, atezolizumab, and durvalumab) with positive immune checkpoint agonist antibodies such as agonist antibodies to 4-1BB (relumab, clinical trial identifier NCT02253992), agonist antibodies to ICOS (e.g. JTX-2011, e.g. clinical trial identifier NCT02904226), agonist antibodies to CD27 (e.g., varlilumab, e.g., clinical trial identifier NCT02335918), agonist antibodies to GITR (e.g., GWN323, e.g., clinical trial identifer NCT02740270), and agonist antibodies to OX40 (e.g., MEDI6383, (e.g., clinical trial identifer NCT02221960).
(c) CTLA4 pathway inhibitors (including but not limited to ipilumuab) with LAG3 antagonist antibodies (e.g. BMS-986016); TIM3 antagonist antibodies.
   Other representative combination therapies with PD1/PDL1 pathway inhibitors that may be supplemented by the additional of an IL-10 agent include the combination PD1/PDL1 pathway inhibitors with BRAF/MEK inhibitors, kinase inhibitors such as sunitinib (NCT02484404), PARP inhibitors such as olaparib (NCT02484404) EGFR inhibitors such as osimertinib (Ahn, et al. (2016) J Thorac Oncol 11:S115), IDO inhibitors such as epacadostat, and oncolytic viruses such as talimogene laherparepvec (T-VEC). Other representative combination therapies with CTL4 pathway inhibitors that may be supplemented by the additional of an IL-10 agent include the combination CTL4 pathway inhibitors with IL2, GMCSF and IFN-α.

It should be noted that therapeutic responses to immune checkpoint pathway inhibitors often manifest themselves much later than responses to traditional chemotherapies such as tyrosine kinase inhibitors. In some instance, it can take six months or more after treatment initiation with immune checkpoint pathway inhibitors before objective indicia of a therapeutic response are observed. In addition, in some cases involving anti-CTLA4 antibody therapy, metastatic lesions actually increase in size on computed tomography (CT) or magnetic resonance imaging (MRI) scans before subsequently regressing [See, e.g., Pardoll, (April 2012) Nature Rev. Cancer 12:252-64]. Therefore, a determination as to whether treatment with an immune checkpoint pathway inhibitors(s) in combination with a CAR-T cell and/or IL-10 agent of the present disclosure must be made over a time-to-progression that is frequently longer than with conventional chemotherapies. The desired response can be any result deemed favorable under the circumstances. In some embodiments, the desired response is prevention of the progression of the disease, disorder or condition, while in other embodiments the desired response is a regression or stabilization of one or more characteristics of the disease, disorder or conditions (e.g., reduction in tumor size). In still other embodiments, the desired response is reduction or elimination of one or more adverse effects associated with one or more agents of the combination.

### 3. Chemokine and Cytokine Agents as Supplementary Agents:

In an alternative to co expression on the CAR vector, cytokines such as IL-2, IL-7, IL-12, IL-15 and IL18, as well as analogs and variants thereof, may be administered as supplementary agents with CAR-T cell therapy. Examples of additional supplementary agents include but are not limited to IL-7 agents, modified polypeptide IL-10 agents, modified polypeptide IL-12 agents, modified polypeptide IL-7 agents, modified polypeptide IL-15 agents, PEGylatedIL-2 agents and modified polypeptide IL-18 agents, specifically including PEGylated IL-7 agents, PEGylated IL-12 agents, PEGylated IL-7 agents, PEGylated IL-15 agents (in particular those disclosed in McCauley, et al PCT Application No. PCT/US2016/067042, international publication WO 2017/112528 published Jun. 29, 2017), PEGylated IL-2 agents (including but not limited to NKTR-214, Nektar Therapeutics, Inc.), PEGylated IL-18 agents, IL-7 variants, IL-10 variants, IL-12 variants, IL-7 variants, IL-15 variants, IL-2 variants, IL-18 variants, IL-7 analogs, IL-10 analogs, IL-12 analogs, IL-7 analogs, IL-15 analogs, IL-2 analogs, and IL-18 analogs.

In some embodiments the PEGylated IL-15 molecule has the structure:

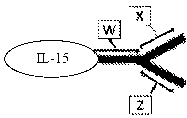

wherein w, x and z are PEG molecules and the MW of each of x, w and z is the same, the MW of at least one of x, w and z is different, the MW of each of x and z is the same, and wherein the MW of each of x and z is different. The present disclosure contemplates embodiments wherein the MW of the PEG is from 7.5 kDa to 80 kDa, is from 15 kDa to 45 kDa, is from 15 kDa to 60 kDa, is from 15 kDa to 80 kDa, is from 20 kDa to 30 kDa, is from 20 kDa to 40 kDa, is from 20 kDa to 60 kDa, is from 20 kDa to 80 kDa, is from 30 kDa to 40 kDa, is from 30 kDa to 50 kDa, is from 30 kDa to 60 kDa, is from 30 kDa to 80 kDa, is from 40 kDa to 60 kDa, or is from 40 kDa to 80 kDa. In particular embodiments, the MW of each of x and z is 20 kDa, and the MW of w is 10 kDa.

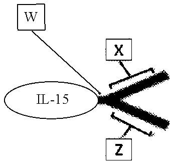

wherein x and z are PEG molecules, wherein x and z represent components of a PEG, and the IL-15 is covalently attached to the PEG via a linker w which may also be a PEG molecule. In certain embodiments, the MW of the PEG x or z PEG is about 20 kDa, about 30 kDa, about 40 kDa, about 50 kDa, about 60 kDa, about 70 kDa, or about 80 kDa or more. Particular embodiments are contemplated wherein the MW of each of x and z is 10 kDa, 20 kDa, 30 kDa, or 40 kDa.

### V. Activation-Induced Cell Death

The infusion of genetically-modified T-cells (such as car T-cells) directed to specific target antigens has several potential benefits, including long-term disease control, rapid onset of action similar to that of cytotoxic chemotherapy or with targeted therapies, and circumvention of both immune tolerance of the T-cell repertoire and MHC restriction. However, treatment of certain cancers (e.g., non-B cell malignancies) with CAR-T cell therapy has, in part, been limited by both the induction of antigen-specific toxicities targeting normal tissues expressing the target-antigen, and the extreme potency of CAR-T cell treatments, sometimes resulting in life-threatening cytokine-release syndromes (Magee (November 2014) Discov Med 18(100):265-71). In particular, it has been observed that high affinity T-cell receptor interactions with significant antigen burden can lead to activation-induced cell death (Song et al. (2012) Blood 119(3):696-706; Hombach et al (2013) Mol Ther 21(12):2268-77).

Activation-induced cell death (AICD), programmed cell death that results from the interaction of Fas receptors (e.g., Fas, CD95) with Fas ligands (e.g., FasL, CD95 ligand), helps to maintain peripheral immune tolerance. The AICD effector cell expresses FasL, and apoptosis is induced in the cell expressing the Fas receptor. Activation-induced cell death is a negative regulator of activated T lymphocytes resulting from repeated stimulation of their T-cell receptors. Alteration of this process may lead to autoimmune diseases (Zhang J, et al. (2004) Cell Mol Immunol. 1(3):186-92).

Mechanistically, the binding of a Fas ligand to a Fas receptor triggers trimerization of the Fas receptor, whose cytoplasmic domain is then able to bind the death domain of the adaptor protein FADD (Fas-associated protein with death domain). Procaspase 8 binds to FADD's death effector domain and proteolytically self-activates caspase 8; Fas, FADD, and procaspase 8 together form a death-inducing signaling complex. Activated caspase 8 is released into the cytosol, where it activates the caspase cascade that initiates apoptosis (Nagata S. (1997) Cell. 88(3):355-65s.

Basically, activation induced cell death of car T-cells is a problem that prevents the long-term maintenance of CAR T-cell therapy's effects.

The balance of activation-induced proliferation and death of effecter cells is a key point in the homeostatic expansion of T-cells. While resting T-cells are susceptible to apoptosis, stimulation of T-cells through TCR/CD3 in the presence of cytokines (e.g., IL-2, IL-4, IL-7 and IL-12) results in clonal expansion. Interestingly, the roles of these molecules in the homeostasis of T-cells are sometimes paradoxical. By way of example, IL-2 is necessary for proliferation and survival of CD4+ T-cells, but it is also a prerequisite for activation-induced cell death. Moreover, IL-18 has been shown to promote expansion and survival of activated CD8+ T-cells. IL-18 may influence immune/inflammatory responses by regulating the size of the CD8+ T-cell population with specific functions following exposure to stimuli. Regulation of proliferation and activation-induced cell death of activated T-cells is closely associated with immune/inflammatory responses (Li, W., et al. (July 2007) J Leukocyte Bio 82(1):142-51).

In one embodiment of the invention, the invention provides methods and compositions to inhibit CAR-T cell apoptosis by contacting the CAR-T cell with an IL-10 agent, administering to a subject undergoing CAR-T cell therapy, an IL-10 agent (including PEGylated IL-10 agents) before, during or after the administration of the CAR-T cell therapy, wherein the administration is contemporaneous with the administration of the CAR-T cell agent or within the therapeutic window associated with the CAR-T cell therapy. Additionally, in one embodiment of the invention, the invention provides methods and compositions to inhibit CAR-T cell apoptosis by modifying the CAR-T cell to express a polypeptide IL-10 agent, the modifying being achieve by introducing a vector comprising a nucleic acid sequence capable of directing the expression of the IL-10 polypeptide in the CAR-T cell. In one embodiment, the invention provides a method of inhibiting apoptosis in CAR-T cells ex vivo by contacting the CAR-T cells with an IL-10 agent. In one embodiment, the invention provides compositions and methods to extend the lifespan of CAR-T cells ex vivo by suspending the CAR-T cells in a solution containing an IL-10 agent.

### W. Effect of IL-10 on CAR-T Cell Therapy

The characteristics of IL-10 agents (e.g., PEG-IL-10) are described elsewhere herein. As an anti-inflammatory and immunosuppressive molecule, IL-10 inhibits antigen presentation, CD4+ T-cell function, CD8+ T-cell pathogen-specific function (Biswas et al. (2007) J Immunol 179(7):4520-28), viral epitope-specific CD8+ T-cell IFNγ responses (Liu et al. (2003) J Immunol 171(9):4765-72), and anti-LCMV (Lymphocytic Choriomeningitis Virus) CD8+ T-cell responses (Brooks et al. (2008) PNAS USA 105(51):20428-433).

While IL-10 has been discussed in the context of enhancement of activation-induced cell death (Georgescu et al. (1997) J Clin Invest 100(10):2622-33), in vitro and in vivo data presented herein indicate that an IL-10 agent (e.g., PEG-IL-10) may be combined with CAR-T cell therapy to prevent or limit activation-induced cell death while enhancing CD8+ T-cell function and survival.

By way of example, the findings presented in Example 1 of the Experimental section suggest that PEG-IL-10 administration mediated CD8+ T-cell immune activation. As described in Example 1, the number of PD-1- and LAG3-expressing CD8+ T-cells was compared in oncology patients before and after treatment with PEG-rHuIL-10 (see Example 1). Both PD-1 and LAG3 are markers of CD8+ T-cell activation and cytotoxic function. The number of peripheral CD8+ T-cells expressing PD-1 increased by ~2-fold, and the number of peripheral CD8+ T-cells expressing LAG3 increased by ~4-fold. Taken as a whole, these data indicate that PEG-IL-10 administration mediated CD8+ T-cell immune activation.

Administration of PEG-IL-10 was also observed to enhance the function of activated memory CD8+ T-cells (see Example 2). Memory T-cells (also referred to as antigen-experienced T-cells) are a subset of T lymphocytes (e.g., helper T-cells (CD4+) and cytotoxic T-cells (CD8+)) that have previously encountered and responded to their cognate antigen during prior infection, exposure to cancer, or previous vaccination. In contrast, naïve T-cells have not encountered their cognate antigen within the periphery; they are commonly characterized by the absence of the activation markers CD25, CD44 or CD69, and the absence of memory CD45RO isoform. Memory T-cells, which are generally CD45RO+, are able to reproduce and mount a faster and stronger immune response than naïve T-cells.

As discussed, CAR-T cells are frequently derived from memory CD8+ T-cells, the effect of PEG-IL-10 on memory CD8+ T-cells was assessed in vitro. The data presented in Example 2 are consistent with the effect of PEG-IL-10 to enhance the function of activated memory CD8+ T-cells.

To evaluate the effects of the administration of an IL-10 agent in combination with CAR-T cells, an in vitro study was performed to assess the impact of an IL-10 agent on cytoxicity, IFNγ release and Granzyme B induction in CAR-T cells exposed to target tumor cells as more fully described in the Examples hereunder. As noted, the IL-10 agent employed in these experiments was AM0010, an approximately 50/50 mixture of monopegylated and dipegylated recombinant human IL-10. The CAR-T cells used in these experiments were CD8+ T cells transduced with a recombinant lentiviral vector encoding an anti-CD-19 CD28-CD3z chimeric antigen receptor (CAR). The target cells were CD19+ HeLa human cervical cancer cells. Approximately 10,000 CD19/HeLa target cells were added to each well of an E-plate microtiter plate (commercially available from ACEA Biosciences). Cells were allowed and allowed to expand for a period of approximately 24 hours to reach confluence. Anti-CD-19 CD28-CD3z CAR-T cells were prepared using human PBMCs obtained from a blood bank which were then transfected with a recombinant lentiviral vector expressing a nucleic acid construct encoding anti-CD-19 CD28-CD3z chimeric antigen receptor. Anti-CD-19 CD28-CD3z CAR-T cells were to each well added (in triplicate) at varying Effector:Target (E:T) ratios of anti-CD19 CAR-T effector cells to CD19/HeLa target cells in the following amounts: (a) 100,000 CAR-T Cells (10:1 E:T ratio); (b) 50,000 CAR-T Cells (5:1 E:T ratio); (c) 20,000 CAR-T Cells (2:1 E:T ratio); and (e) 10,000 CAR-T Cells (1:1 E:T ratio). The IL-10 agent AM0010 was added to each well at four concentrations, 1000 ng/ml, 100 ng/ml, 10 ng/ml, 1 ng/ml with a control well with no AM-0010 during the course of exposure to the HeLa cells to the anti-CD19 CAR-T cells, with respect to each E:T ratio. The effect of the CAR-T cells on cytotoxicity, IFNγ induction and granzyme B release in the absence of the IL-10 agent AM0010 was also evaluated. As a control, the effect on cytotoxicity, IFNγ induction and granzyme B release of the non-transduced T-cells in the presence and absence of the IL-10 agent AM0010 was also evaluated at two E:T ratios, 2:1 and 10:1.

The exposure of the target CD19-HeLa cells in the presence of IL-10 resulted in increased secretion of granzyme B in an IL-10 dose dependent fashion when looking at granzyme B induction response of target CD19-HeLa cells in response to varying E:T ratios of anti-CD19 CD28-CD3z CAR-T cells in the presence of varying concentrations of AM-0010 without pretreatment with IL-10. Granzyme B was measured 8 and 24 hours after addition of the CAR-T cells using a commercially available sandwich ELISA assay kit catalog #DY2906-05 (commercially available from R&D Systems, 614 McKinley Place NE, Minneapolis, MN 55413) in substantial accordance with the instructions provided by the manufacturer.

IFNγ, a hallmark of immune activation and correlative of anti-tumor immune response, was measured at 8 and 24 hours after addition of the CAR-T cells using a conventional sandwich ELISA assay kit catalog #KHC4012 (commercially available ThermoFisher Scientific 168 Third Avenue Waltham, MA USA 02451) in substantial accordance with the instructions provided by the manufacturer. Data resulting from an in vitro analysis of the interferon-gamma induction response of target CD19-HeLa cells in response to varying E:T ratios of anti-CD19 CD28-CD3z CAR-T cells in the presence of varying concentrations of AM-0010 without pretreatment with IL-10 as more fully described in the Examples illustrate the exposure of the target CD19-HeLa cells in the presence of IL-10 resulted in increased secretion of interferon-gamma expression, a hallmark of T-cell activation, in an IL-10 dose dependent fashion.

Cytotoxicity was evaluated approximately every five minutes over a period of approximately 25 hours following administration of the CAR-T cells using the ACEA xCelligence® Real Time Cell Analysis (RTCA) system (ACEA Biosciences, Inc., San Diego CA). In this system, the adherent target cells are seeded into the wells of a multi-well electronic microtiter plate (“E-plate”) providing an array of gold microelectrodes. As cells proliferate across the surface, the electrical impedance across the electrode array increases. As cells die and lift from the plate causing a reduction in electrical impedance. Thus, by measuring the impedance of electron flow across the array, one is able to measure viability of the cells in real time. The impedance of electron flow caused by the adherent cells is reported as Cell Index (CI), a unitless parameter calculated as: Cell Index (CI)=(impedance at time point n−impedance in the absence of cells)/nominal impedance value. As adherent cells proliferate across the surface of the plate, the CI rises reflecting an increase in electrical impedance. When the CI plateaus, the cells are presumed to be confluent on the plate. As the adherent target cells die, they lift from the electronic microtiter well surface resulting in a reduction in electrical impedance (increased conductivity) which can be measured for each plate enabling continuous evaluation of cytotoxicity over time. The electrical resistance data was collected every 5 minutes during the course of the experiment and the data analyzed using the software provided with the xCELLigence® system. The data from each triplicate well was combined and averaged using the same software.

Results obtained from this study demonstrate that the addition of an IL-10 agent to CAR-T cells mediated specific enhancement of CAR-T cytotoxicity in an IL-10 agent dose dependent fashion. In particular, a comparison of the data demonstrates that the significant enhancement of target cell cytotoxicity in the presence of an IL-10 agent. In particular, the enhanced cytotoxic effect of the CAR-T cells against the target neoplastic cells is observed even a very low concentrations of IL-10 (0.1 ng/ml). Consequently, administration of IL-10 agents to achieve a serum trough concentration of less than about 0.1 ng/ml, alternatively less than about 0.08 ng/ml, alternatively less than about 0.06 ng/ml, alternatively less than about 0.05 ng/ml, alternatively less than about 0.03 ng/ml, alternatively less than about 0.01 ng/ml would be useful in enhancing the therapeutic effect of (or reducing the toxicity of) a CAR-T cell therapy. As previously discussed, some CAR-T cell therapies have been associated with significant adverse events in the treatment of human subjects. This data also demonstrates that the combination of a CAR-T cell therapy (particularly CAR-T cell therapies possessing serious side effects including “black box” warnings) with an IL-10 agent facilitates the administration of a lower dose (administration of a lower number of cells, administration at a lower E:T ratio) of CAR-T cells thereby achieving a reduction of adverse events, particularly serious adverse events, associated with the CAR-T cell therapy while providing a therapeutic benefit to a subject comparable to that observed a higher CAR-T cell dose. In particular, the enhanced cytotoxic effect of the CAR-T cells against the target neoplastic cells is observed even a very low concentrations of IL-10 (0.1 ng/ml). Consequently, administration of IL-10 agents to achieve a serum trough concentration of the IL-10 agent of less than about 0.1 ng/ml, alternatively less than about 0.08 ng/ml, alternatively less than about 0.06 ng/ml, alternatively less than about 0.05 ng/ml, alternatively less than about 0.03 ng/ml, alternatively less than about 0.01 ng/ml would be useful in enhancing the therapeutic effect of (and/or reducing the toxicity of) a CAR-T cell therapy.

The cytotoxicity data obtained from the foregoing experiment was replotted as histograms demonstrating the enhanced cytotoxic effect on a culture of 10,000 CD19/HeLa cells by the addition an IL-10 agent (AM0010) at varying concentrations (0 ng/ml, 1 ng/ml, 10 ng/ml, 100 ng/ml and 1000 ng/ml) as indicated in combination with and varying amounts of anti-CD-19 CAR-T cells. The addition of AM0010 enhanced the cytotoxic effect of anti-CD-19 CAR-T cells on CD19/HeLa cells at all ratios of anti-CD-19 CAR-T to CD19/HeLa cells at all tested concentrations of AM-0010.

A further study was performed to evaluate the effect of pre-treatment of CAR T cells with an IL-10 agent ex vivo prior to implantation in the subject. CAR-T cell cytotoxicity to CD19-HeLa target tumor cells was evaluated as described above with in response to varying E:T ratios of anti-CD19 CD28-CD3z CAR-T cells in the presence of varying concentrations of AM-0010 8 and 24 hours after administration of the CAR-T cells wherein the CAR-T cells were pre-incubated with IL-10 prior to exposure to the target cells as more fully described in the Examples. The exposure of the target CD19-HeLa cells in the presence of IL-10 resulted in increased cytotoxicity of the CAR-T cells in an IL-10 dose dependent fashion at the 8 hour time point. Further, the exposure of the target CD19-HeLa cells in the presence of IL-10 resulted in increased cytotoxicity of the CAR-T cells in an IL-10 dose dependent fashion. The exposure of the target CD19-HeLa cells in the presence of IL-10 resulted in increased IFNγ expression, a hallmark of T-cell activation and antitumor effect, in an IL-10 dose dependent fashion.

In addition to the foregoing in vitro study, an additional in vivo study was conducted to evaluate the effect the combination of an IL-10 agent (AM-0010) with anti-tumor CAR-T cell therapy in an in vivo tumor model of neoplastic disease in mice. Briefly, cohorts of 5 Female NOD.Cg-Prkdcscid IL2rgtm1Wjl/SzJ (NOD/scid IL2RGnull) mice were inoculated intraperitoneally with 0.5×10⁶ Raji-luc cells, a CD19+Raji human Burkitt's lymphoma cell line constructed by engineering the Raji cell line (ATCC CCL-86) by transduction with a vector providing the luciferase gene enabling full body bioluminescence to evaluate tumor growth.

From the results of the in vivo analysis of CAR-T cell activity in a mouse cancer model as more fully described in the examples, especially Example 17. Controls (no therapy) resulted in rapid mortality, all mice being dead by day 21 of the study demonstrating the rapid progression of the disease in the animals. The effects of AM0010 alone, non-transduced (i.e. non-CAR) T cells without AM-0010 or with AM-0010 provided some anti-tumor effect but still with significant mortality with multiple deaths of animals by day 35 of the study. The whole-body bioluminescence imaging data demonstrates that tumors (the dark areas) spread rapidly through the animals resulting in morbidity and mortality to all animals in the cohort resulting in death with multiple deaths of animals by day 35 of the study.

The administration of 5 million CAR-T cells demonstrated some therapeutic benefit with all 5 animals surviving to day 35 of the study. However, the results of the exposure of the mice to 0.5 mg/kg AM-0010 in combination with the administration of 5 million CAR-T cells demonstrates significant improvement of CAR-T cell therapy when administered in the presence of an IL-10 agent over CAR-T cell therapy alone, the combination demonstrating a significant tumor reduction in the majority of animals. A similar experiment with a lower (2.5 million) quantity of CAR-T cells was performed with without the administration of 0.5 mg/kg AM-0010 to the mice. The exposure of the mice to 2.5 million CAR-T cells exhibited some therapeutic benefit with all 5 animals surviving to day 35 of the study. However, contrasting these data to the therapeutic effect in conjunction with the administration of an IL-10 agent demonstrates a significant improvement of CAR-T cell therapy when administered in the presence of an IL-10 agent over CAR-T cell therapy alone, the combination demonstrating a significant tumor reduction in the majority of animals.

These results are confirmed by whole-body bioluminescence data. The bioluminescence data generated associated with the administration of 5 million CAR-T cells indicating some therapeutic benefit with all 5 animals surviving to day 35 of the study. Whole-body bioluminescence data generated resulting from treatment with 0.5 mg/kg AM-0010 with 5 million CAR-T cells provides a significant therapeutic improvement to CAR-T cell therapy when administered in the presence of an IL-10 agent over CAR-T cell therapy alone, the combination demonstrates a significant tumor reduction and apparent absence of tumor in three of 5 animals at day 35.

Whole-body bioluminescence data resulting from treatment with 2.5 million CAR-T cells exhibited some therapeutic benefit with all 5 animals surviving to day 35 of the study. Whole-body bioluminescence data associated with the combined treatment regiment of 0.5 mg/kg AM-0010 and 2.5 million CAR-T cells demonstrates a significant benefit of the combined treatment over CAR-T cell therapy alone.

The foregoing in vivo data demonstrates in an art recognized tumor model of the enhanced anti-tumor effect provided by combining CAR-T cell therapy with the administration of an IL-10 agent.

### X. Pharmaceutical Compositions

When a CAR-T cell and/or IL-10 agent is administered to a subject, the present disclosure contemplates the use of any form of compositions suitable for administration of such agents to the subject. In general, such compositions are “pharmaceutical compositions” comprising CAR-T cell and/or IL-10 agent and one or more pharmaceutically acceptable or physiologically acceptable diluents, carriers or excipients as well as, optionally, supplementary therapeutic agents. The pharmaceutical compositions can be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Furthermore, the pharmaceutical compositions can be used in combination with other therapeutically active agents or compounds as described herein in order to treat or prevent the diseases, disorders and conditions as contemplated by the present disclosure.

The pharmaceutical compositions typically comprise a therapeutically effective amount of an IL-10 agent contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle can be a physiological saline solution or citrate buffered saline, possibly supplemented with other materials common in pharmaceutical compositions for parenteral administration. Neutral buffered saline or saline mixed with serum albumin are further exemplary vehicles.

Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it can be stored in sterile vials as a solution, suspension, gel, emulsion, solid, or dehydrated or lyophilized powder. Such formulations can be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus can be used to deliver IL-10, including implants (e.g., implantable pumps) and catheter systems, slow injection pumps and devices, all of which are well known to the skilled artisan. Depot injections, which are generally administered subcutaneously or intramuscularly, can also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions can be in the form of a sterile injectable aqueous or oleagenous suspension. This suspension can be formulated according to the known art using those suitable dispersing or wetting agents and suspending agents mentioned herein. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil can be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions containing the active ingredient can be in a form suitable for oral use, for example, as tablets, capsules, troches, lozenges, aqueous or oily suspensions, dispersible powders or granules, emulsions, hard or soft capsules, or syrups, solutions, microbeads or elixirs. In particular embodiments, an active ingredient of an agent co-administered with an IL-10 agent described herein is in a form suitable for oral use. Pharmaceutical compositions intended for oral use can be prepared according to any method known to the art for the manufacture of pharmaceutical compositions, and such compositions can contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients can be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions can also contain one or more preservatives.

Oily suspensions can be formulated by suspending the active ingredient in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure can also be in the form of oil-in-water emulsions. The oily phase can be a vegetable oil, for example olive oil or arachis oil, or a mineral oil, for example, liquid paraffin, or mixtures of these. Suitable emulsifying agents can be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed.

The present disclosure contemplates the administration of the IL-10 polypeptides in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The CAR-T cell and IL-10 agents (e.g., PEG-IL-10) and other agents contemplated by the present disclosure can be in the form of any other suitable pharmaceutical composition (e.g., sprays for nasal or inhalation use) currently known or developed in the future.

The concentration of a polypeptide (e.g., IL-10) or fragment thereof in a formulation can vary widely (e.g., from less than about 0.1%, usually at or at least about 2% to as much as 20% to 50% or more by weight) and will usually be selected primarily based on fluid volumes, viscosities, and subject-based factors in accordance with, for example, the particular mode of administration selected.

The IL-10 agents and CAR-T cell (as well as supplementary agents for administration in combination with the IL-10/CAR-T cell therapy) of the present disclosure can be in the form of compositions suitable for administration to a subject. In general, such compositions are “pharmaceutical compositions” comprising IL-10 and/or a CAR-T cell, and one or more pharmaceutically acceptable or physiologically acceptable diluents, carriers or excipients. In certain embodiments, the IL-10 agents and CAR-T cell are each present in a therapeutically acceptable amount. In those embodiments of the invention, where the CAR-T cells are pre-incubated ex vivo with an IL-10 agent, the CAR-T cells may be administered in conjunction with the pre-incubation IL-10 agent without the need to remove the IL-10 agent from the CAR-T cells prior to administration. The pharmaceutical compositions can be used in the methods of the present disclosure; thus, for example, the pharmaceutical compositions can be administered ex vivo or in vivo to a subject in order to practice the therapeutic and prophylactic methods and uses described herein.

In one embodiment, the present invention provides a pharmaceutically acceptable formulation comprising an IL-10 agent and a CAR-T cell. In one embodiment, the pharmaceutically acceptable formulation comprising a pharmaceutically acceptable formulation comprising an IL-10 agent and a CAR-T cell is frozen. In one embodiment, the pharmaceutically acceptable formulation is prepared by thawing a quantity of CAR-T cells and contacting the thawed CAR-T cells with a pharmaceutically acceptable formulation comprising an IL-10 agent. In one embodiment, the acceptable formulation comprising a pharmaceutically acceptable formulation comprising an IL-10 agent and a CAR-T cell is prepared within 24 hours prior to administration to the subject, optionally within 12 hours of administration to the subject, optionally within 8 hours of administration to the subject, optionally within 6 hours of administration to the subject, optionally within 4 hours of administration to the subject, optionally within 2 hours of administration to the subject, optionally within 1 hour of administration to the subject, or optionally within 30 minutes of administration to the subject. In one embodiment of the invention, the invention provides a method of treatment of a disease, disorder or condition by the administration of a pharmaceutical formulation comprising a CAR-T cell and an IL-10 agent. In one embodiment of the invention, the invention provides a method of treatment of a disease, disorder or condition by the administration of a pharmaceutical formulation comprising a CAR-T cell and an IL-10 agent wherein the pharmaceutically acceptable formulation comprising an IL-10 agent and a CAR-T cell is prepared within 24 hours prior to administration to the subject, optionally within 12 hours of administration to the subject, optionally within 8 hours of administration to the subject, optionally within 6 hours of administration to the subject, optionally within 4 hours of administration to the subject, optionally within 2 hours of administration to the subject, optionally within 1 hour of administration to the subject, or optionally within 30 minutes of administration to the subject. In one embodiment, the disease disorder or condition to be treated is selected from the group consisting of neoplastic, inflammatory, or hyperproliferative diseases, disorder or conditions.

### Y. Routes of Administration

The present disclosure contemplates the administration of the CAR-T cell and IL-10 agent (e.g., PEG-IL-10), and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation. Depot injections, which are generally administered subcutaneously or intramuscularly, can also be utilized to release the IL-10 agents disclosed herein over a defined period of time.

In some particular embodiments of the present disclosure, the CAR-T cell and IL-10 agents (e.g., PEG-IL-10) are administered parenterally, and in further particular embodiments the parenteral administration is subcutaneous. In some embodiment, the CAR-T cells is provided intravenously and the IL-10 agent is administered subcutaneously.

As to the CAR-T cell therapy, described herein are alternative means for introducing to a subject a therapeutically effective plurality of cells genetically modified to express a chimeric antigen receptor, wherein the chimeric antigen receptor comprises at least one antigen-specific targeting region capable of binding to the target cell population, and wherein the binding of the chimeric antigen receptor targeting region to the target cell population is capable of eliciting activation-induced cell death.

### Z. Kits

The present disclosure also contemplates kits comprising CAR-T cell and an IL-10 agent (e.g., PEG-IL-10), and a pharmaceutical composition thereof. The kits are generally in the form of a physical structure housing various components, as described below, and can be utilized, for example, in practicing the methods described above. A kit can include a CAR-T cell and an IL-10 agent (e.g., PEG-IL-10) disclosed herein (provided in, e.g., a sterile container), which can be in the form of a pharmaceutical composition suitable for administration to a subject. The CAR-T cell and an IL-10 agent (e.g., PEG-IL-10) IL-10 agent can be provided in a form that is ready for use or in a form requiring, for example, thawing, reconstitution or dilution prior to administration. When the CAR-T cell and/or the IL-10 agent is in a form that needs to be reconstituted by a user, the kit can also include buffers, pharmaceutically acceptable excipients, and the like, packaged with or separately from the IL-10 agent. A kit can also contain both the IL-10 agent and/or components of the specific CAR-T cell therapy to be used; the kit can contain the several agents separately or they can already be combined in the kit. A kit of the present disclosure can be designed for conditions necessary to properly maintain the components housed therein (e.g., refrigeration or freezing).

A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.). Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via an internet site, including by secure access by providing a password (or scannable code such as a barcode or QR code on the container of the IL-10 or CAR-T cells) to comply with governmental regulations (e.g HIPAA) are provided.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that can be performed. It is to be understood that exemplary descriptions written in the present tense were not necessarily performed, but rather that the descriptions can be performed to generate the data and the like described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (°C), and pressure is at or near atmospheric. Standard abbreviations are used, including the following: s or sec=second(s); min=minute(s); h or hr=hour(s); aa=amino acid(s); bp=base pair(s); kb=kilobase(s); nt=nucleotide(s); ng=nanogram; g=microgram; mg=milligram; g=gram; kg=kilogram; dl or dL=deciliter; μl or μL=microliter; ml or mL=milliliter; l or L=liter; nM=nanomolar; μM=micromolar; mM=millimolar; M=molar; kDa=kilodalton; i.m.=intramuscular(ly); i.p.=intraperitoneal(ly); SC or SQ=subcutaneous(ly); HPLC=high performance liquid chromatography; BW=body weight; U=unit; ns=not statistically significant; PMA=Phorbol 12-myristate 13-acetate; PBS=phosphate-buffered saline; DMEM=Dulbeco's Modification of Eagle's Medium; PBMCs=primary peripheral blood mononuclear cells; FBS=fetal bovine serum; FCS=fetal calf serum; HEPES=4-(2-hydroxyethyl)-1-piperazineethanesulfonic acid; LPS=lipopolysaccharide; RPMI=Roswell Park Memorial Institute medium; APC=antigen presenting cells; FACS=fluorescence-activated cell sorting.

The following general materials and methods were used, where indicated, or may be used in the Examples below:

Molecular Biology Procedures. Standard methods in molecular biology are described in the scientific literature (see, e.g., Sambrook and Russell (2001) Molecular Cloning, 3^{rd} ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

Antibody-related Processes. Production, purification, and fragmentation of polyclonal and monoclonal antibodies are described (e.g., Harlow and Lane (1999) Using Antibodies, Cold Spring Harbor Laboratory Press, Cold Spring Harbor, NY); standard techniques for characterizing ligand/receptor interactions are available (see, e.g., Coligan et al. (2001) Current Protocols in Immunology, Vol. 4, John Wiley, Inc., NY); methods for flow cytometry, including fluorescence-activated cell sorting (FACS), are available (see, e.g., Shapiro (2003) Practical Flow Cytometry, John Wiley and Sons, Hoboken, NJ); and fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR.; Sigma-Aldrich (2003) Catalogue, St. Louis, MO.). Further discussion of antibodies appears elsewhere herein.

Software. Software packages and databases for determining, e.g., antigenic fragments, leader sequences, protein folding, functional domains, glycosylation sites, and sequence alignments, are available (see, e.g., GCG Wisconsin Package (Accelrys, Inc., San Diego, CA); and DeCypher™ (TimeLogic Corp., Crystal Bay, NV).

Pegylation. Pegylated IL-10 as described herein may be synthesized by any means known to the skilled artisan. Exemplary synthetic schemes for producing mono-PEG-IL-10 and a mix of mono-/di-PEG-IL-10 have been described (see, e.g., U.S. Pat. No. 7,052,686; US Pat. Publn. No. 2011/0250163; WO 2010/077853). Particular embodiments of the present disclosure comprise a mix of selectively pegylated mono- and di-PEG-IL-10. In addition to leveraging her own skills in the production and use of PEGs (and other drug delivery technologies) suitable in the practice of the present disclosure, the skilled artisan is familiar with many commercial suppliers of PEG-related technologies (e.g., NOF America Corp (Irvine, CA) and Parchem (New Rochelle, NY)).

Animals. Various mice and other animal strains known to the skilled artisan can be used in conjunction with the teachings of the present disclosure. For example, immunocompetent Balb/C or B-cell—deficient Balb/C mice can be obtained from The Jackson Lab., Bar Harbor, ME and used in accordance with standard procedures (see, e.g., Martin et al (2001) Infect. Immun., 69(11):7067-73 and Compton et al. (2004) Comp. Med. 54(6):681-89).

IL-10 Concentrations. Serum IL-10 concentration levels and exposure levels can be determined by standard methods used in the art. For example, when the experimental subject is a mouse, a serum exposure level assay can be performed by collecting whole blood (~50 μL/mouse) from mouse tail snips into plain capillary tubes, separating serum and blood cells by centrifugation, and determining IL-10 exposure levels by standard ELISA kits and techniques.

FACS Analysis. Numerous protocols, materials and reagents for FACS analysis are commercially available and may be used in conjunction with the teachings herein (e.g., Becton-Dickinson, Franklin Lakes, NJ; Cell Signaling Technologies, Danford, MA; Abcam, Cambridge, MA; Affymetrix, Santa Clara, CA). Both direct flow cytometry (i.e., using a conjugated primary antibody) and indirect flow cytometry (i.e., using a primary antibody and conjugated secondary antibody) may be used. An exemplary direct flow protocol is as follows: Wash harvested cells and adjust cell suspension to a concentration of 1-5×10⁶ cells/mL in ice-cold PBS, 10% FCS, 1% sodium azide. Cells may be stained in polystyrene round bottom 12×75 mm² Falcon tubes. Cells may be centrifuged sufficiently so the supernatant fluid may be removed with little loss of cells, but not to the extent that the cells are difficult to resuspend. The primary labeled antibody may be added (0.1-10 g/mL), and dilutions, if necessary, may be made in 3% BSA/PBS. After incubation for at least 30 min at 4 °C, cells may be washed 3× by centrifugation at 400 g for 5 min and then may be resuspended in 0.5-1 mL of ice-cold PBS, 10% FCS, 1% sodium azide. Cells may be maintained in the dark on ice until analysis (preferably within the same day). Cells may also be fixed, using standard methodologies, to preserve them for several days; fixation for different antigens may require antigen-specific optimization.

PBMC and CD8+ T-cell Gene Expression Assay. The following protocol provides an exemplary assay to examine gene expression. Human PBMCs can be isolated according to any standard protocol (see, e.g., Fuss et al. (2009) Current Protocols in Immunology, Unit 7.1, John Wiley, Inc., NY). 2.5 mL of PBMCs (at a cell density of 8 million cells/mL) can be cultured per well with complete RPMI, containing RPMI (Life Technologies; Carlsbad, CA), 10 mM HEPES (Life Technologies; Carlsbad, CA), 10% FCS (Hyclone Thermo Fisher Scientific; Waltham, MA) and Penicillin/Streptomycin cocktail (Life Technologies; Carlsbad, CA), in any standard tissue culture treated 6-well plate (BD; Franklin Lakes, NJ). Human pegylated-IL-10 can be added to the wells at a final concentration of 100 ng/mL, followed by a 7-day incubation. CD8+ T-cells can be isolated from the PBMCs using Miltenyi Biotec's MACS cell separation technology according to the manufacturer's protocol (Miltenyi Biotec; Auburn, CA). RNA can be extracted and cDNA can be synthesized from the isolated CD8+ T-cells and the CD8+ T-cell depleted-PBMCs using Qiagen's RNeasy Kit and RT² First Strand Kit, respectively, following the manufacturer's instructions (Qiagen N.V.; Netherlands). Quantitative PCR can be performed on the cDNA template using the RT² SYBR Green qPCR Mastermix and primers (IDO1, GUSB, and GAPDH) from Qiagen according to the manufacturer's protocol. IDO1 Ct values can be normalized to the average Ct value of the housekeeping genes, GUSB and GAPDH.

PBMC and CD8+ T-cell Cytokine Secretion Assay. Activated primary human CD8+ T-cells secrete IFN-γ when treated with PEG-IL-10 and then with an anti-CD3 antibody. The following protocol provides an exemplary assay to examine cytokine secretion.

TNFα Inhibition Assay. PMA-stimulation of U937 cells (lymphoblast human cell line from lung available from Sigma-Aldrich (#85011440); St. Louis, MO) causes the cells to secrete TNFα, and subsequent treatment of these TNFα-secreting cells with human IL-10 causes a decrease in TNFα secretion in a dose-dependent manner. An exemplary TNFα inhibition assay can be performed using the following protocol.

After culturing U937 cells in RMPI containing 10% FBS/FCS and antibiotics, plate 1×105, 90% viable U937 cells in 96-well flat bottom plates (any plasma-treated tissue culture plates (e.g., Nunc; Thermo Scientific, USA) can be used) in triplicate per condition. Plate cells to provide for the following conditions (all in at least triplicate; for ‘media alone’ the number of wells is doubled because one-half will be used for viability after incubation with 10 nM PMA): 5 ng/mL LPS alone; 5 ng/mL LPS+0.1 ng/mL rhIL-10; 5 ng/mL LPS+1 ng/mL rhIL-10; 5 ng/mL LPS+10 ng/mL rhIL-10; 5 ng/mL LPS+100 ng/mL rhIL-10; 5 ng/mL LPS+1000 ng/mL rhIL-10; 5 ng/mL LPS+0.1 ng/mL PEG-rhIL-10; 5 ng/mL LPS+1 ng/mL PEG-rhIL-10; 5 ng/mL LPS+10 ng/mL PEG-rhIL-10; 5 ng/mL LPS+100 ng/mL PEG-rhIL-10; and 5 ng/mL LPS+1000 ng/mL PEG-rhIL-10. Expose each well to 10 nM PMA in 200 μL for 24 hours, culturing at 37 °C in 5% CO₂ incubator, after which time ~90% of cells should be adherent. The three extra wells can be re-suspended, and the cells are counted to assess viability (>90% should be viable). Wash gently but thoroughly 3× with fresh, non-PMA—containing media, ensuring that cells are still in the wells. Add 100 μL per well of media containing the appropriate concentrations (2× as the volume will be diluted by 100%) of rhIL-10 or PEG-rhIL-10, incubate at 37 °C in a 5% CO₂ incubator for 30 minutes. Add 100 μL per well of 10 ng/mL stock LPS to achieve a final concentration of 5 ng/mL LPS in each well, and incubate at 37 °C in a 5% CO₂ incubator for 18-24 hours. Remove supernatant and perform TNFα ELISA according to the manufacturer's instructions. Run each conditioned supernatant in duplicate in ELISA.

MC/9 Cell Proliferation Assay. IL-10 administration to MC/9 cells (murine cell line with characteristics of mast cells available from Cell Signaling Technology; Danvers, MA) causes increased cell proliferation in a dose-dependent manner. Thompson-Snipes, L. et al. (1991) J. Exp. Med. 173:507-10) describe a standard assay protocol in which MC/9 cells are supplemented with IL3+IL-10 and IL-3+IL-4+IL-10. Vendors (e.g., R&D Systems, USA; and Cell Signaling Technology, Danvers, MA) use the assay as a lot release assay for rhIL-10. Those of ordinary skill in the art will be able to modify the standard assay protocol described in Thompson-Snipes, L. et al, such that cells are only supplemented with IL-10.

Activation-induced Cell Death Assay. The following protocol provides an exemplary activation-induced cell death assay.

Human PBMCs can be isolated according to any standard protocol (see, e.g., Fuss et al. (2009) Current Protocols in Immunology, Unit 7.1, John Wiley, Inc., NY). CD8+ T cells (CD45RO+) can be isolated using Miltenyi Biotec's anti-CD45RO MACS beads and MACS cell separation technology according to the manufacture's protocol (Miltenyi Biotec Inc; Auburn, CA). To activate cells, 1 mL of isolated cells (density of 3×10⁶ cells/mL) can be cultured in AIM V media for 3 days (Life Technologies; Carlsbad, CA) in a standard 24-well plate (BD; Franklin Lakes, NJ) pre-coated with anti-CD3 and anti-CD28 antibodies (Affymetrix eBioscience, San Diego, CA). The pre-coating process can be carried out by adding 300 μL of carbonate buffer (0.1 M NaHCO₃(Sigma-Aldrich, St. Louis, MO), 0.5 M NaCl (Sigma-Aldrich), pH 8.3) containing 10 μg/mL anti-CD3 and 2 μg/mL anti-CD28 antibodies to each well, incubating for 2 hours at 37 °C, and washing each well with AIM V media. Following the 3-day activation period, cells can be collected, counted, re-plated in 1 mL of AIM V media (density of 2×10⁶ cells/mL) in a standard 24-well plate and treated with 100 ng/mL PEG-hIL-10 for 3 days. The process of activation and treatment with PEG-hIL-10 can be repeated, after which viable cells can be counted by Trypan Blue exclusion according to the manufacturer's protocol (Life Technologies).

Tumor Models and Tumor Analysis. Any art-accepted tumor model, assay, and the like can be used to evaluate the effect of the IL-10 agents described herein on various tumors. The tumor models and tumor analyses described hereafter are representative of those that can be utilized. Syngeneic mouse tumor cells are injected subcutaneously or intradermally at 10⁴, 10⁵ or 10⁶ cells per tumor inoculation. Ep2 mammary carcinoma, CT26 colon carcinoma, PDV6 squamous carcinoma of the skin and 4T1 breast carcinoma models can be used (see, e.g., Langowski et al. (2006) Nature 442:461-465). Immunocompetent Balb/C or B-cell deficient Balb/C mice can be used. PEG 10-mIL-10 can be administered to the immunocompetent mice, while PEG-hIL-10 treatment can be in the B-cell deficient mice. Tumors are allowed to reach a size of 100-250 mm³ before treatment is started. IL-10, PEG-mIL-10, PEG-hIL-10, or buffer control is administered SC at a site distant from the tumor implantation. Tumor growth is typically monitored twice weekly using electronic calipers. Tumor tissues and lymphatic organs are harvested at various endpoints to measure mRNA expression for a number of inflammatory markers and to perform immunohistochemistry for several inflammatory cell markers. The tissues are snap-frozen in liquid nitrogen and stored at −80 °C. Primary tumor growth is typically monitored twice weekly using electronic calipers. Tumor volume can be calculated using the formula (width²×length/2) where length is the longer dimension. Tumors are allowed to reach a size of 90-250 mm³ before treatment is started.

### Example 1. PEG-IL-10 Mediates CD8+ T-Cell Immune Activation

The change in the number of PD-1- and LAG3-expressing CD8+ T-cells was determined in cancer patients before and after 29 days of treatment with PEG-rHuIL-10. Two patients who responded to the therapy with a sustained partial response had an increase of the PD1+ CD8 T-cells in the blood. The first patient (renal cell carcinoma) received 20 μg/kg PEG-rHuIL-10 SC daily and experienced a 71% reduction of total tumor burden after 22 weeks. The second patient (melanoma) received 40 μg/kg PEG-rHuIL-10 SC daily and experienced a 57% reduction of total tumor burden after 22 weeks.

Peripheral blood monocytic cells (PBMC) were isolated from the periphery of each patient pre-treatment and during the treatment period and were subjected to FACS analysis. The number of peripheral CD8+ T-cells expressing PD-1 increased by ~2-fold within 29 days and continued to increase during the treatment period, and the number of peripheral CD8+ T-cells expressing LAG3 increased by ~4-fold within 29 days. Both PD-1 and LAG3 are markers of CD8+ T-cell activation and cytotoxic function. These findings suggest that PEG-rHuIL-10 administration mediated CD8+ T-cell immune activation.

### Example 2. PEG-IL-10 Enhances the Function of Activated Memory CD8+ T-Cells

Memory T-cells (also referred to as antigen-experienced T-cells) are a subset of T lymphocytes (e.g., helper T-cells (CD4+) and cytotoxic T-cells (CD8+)) that have previously encountered and responded to their cognate antigen during prior infection, exposure to cancer, or previous vaccination. In contrast, naïve T-cells have not encountered their cognate antigen within the periphery; they are commonly characterized by the absence of the activation markers CD25, CD44 or CD69, and the absence of memory CD45RO isoform. Memory T-cells, which are generally CD45RO+, are able to reproduce and mount a faster and stronger immune response than naïve T-cells.

Given that CAR-T cells are derived from memory CD8+ T-cells, the effect of PEG-IL-10 on memory CD8+ T-cells was assessed in vitro using standard methodology, an example of which is described herein. PEG-IL-10 preferentially enhances IFNγ production in memory CD8+ T cells (CD45RO+) and not naïve CD8+ T-cells. These data are consistent with the effect of PEG-IL-10 to enhance the function of activated memory CD8+ T-cells.

### Example 3. PEG-IL-10 Treatment Results in Increased Activated Memory CD8+ T-Cells

As described herein, CAR-T cell therapy is derived from memory CD8+ T-cells. In order to be effective, infused memory CD8+ T-cells must not only exhibit cytotoxicity, but must also persist (Curran K J, Brentjens R J. (20 Apr. 2015) J Clin Oncol pii: JCO.2014.60.3449; Berger et al., (January 2008) J Clin Invest 118(1):294-305). However, repeated activation of T-cells leads to activation-induced cell death, which decreases the number of cells and thus the overall therapeutic efficacy.

Using the procedure described herein, the activation-induced cell death of human CD45RO+ memory CD8+ T-cells from two donors was determined with and without treatment with PEG-IL-10. Treatment of human CD45RO+ memory CD8+ T-cells with PEG-IL-10 after two rounds of TCR and co-stimulation—induced activation resulted in a greater number of viable cells. These data indicate that PEG-IL-10 is capable of limiting activation-induced cell death, thus resulting in a greater number of activated memory T-cells to persist. These observations suggest that the use of PEG-IL-10 in combination with CAR-T cell therapy provides additional clinical benefit.

### Example 4. IL2 Secretion Assay

Levels of secreted IL-2 were determined by use of a human IL-2 ELISA kit (Commercially available as catalog #EH2IL2, ThermoFisher Scientific 168 Third Avenue Waltham, MA USA 02451) in substantial accordance with the manufacturer's instructions.

### Example 5. IFN-Gamma Secretion Assay

Levels of secreted interferon gamma were determined by use of a human IFN-g ELISA kit (catalog #KHC4012, ThermoFisher Scientific 168 Third Avenue Waltham, MA USA 02451) in substantial accordance with the manufacturer's instructions.

### Example 6. Granzyme B Assay

Levels of granzyme B were determined by use of the DuoSet Human Granzyme B ELISA kit (catalog #DY2906-05, R&D Systems 614 McKinley Place NE, Minneapolis, MN 55413, USA) in substantial accordance with the manufacturer's instructions.

### Example 7. FACS—Cell Staining

Cells were washed and suspended in FACS buffer (phosphate-buffered saline (PBS) plus 0.1% sodium azide and 0.4% BSA). Cells were divided into 1×10⁶ aliquots. Fc receptors were blocked with normal goat IgG (LifeTechnologies). 100 μl of 1:1000 diluted normal goat lgG was added to each tube and incubated on ice for 10 min. 1.0 ml FACS buffer was added to each tube, mix well and centrifuged at 300 g for 5 min. Biotin-labeled polyclonal goat anti-mouse-F(ab)2 antibodies (Life Technologies) were added to detect CD19 scFv; biotin-labeled normal polyclonal goat IgG antibodies (Life Technologies) were added to serve as an isotype control. (1:200 dilution, reaction volume of 100 μl).

Cells were incubated at 4 °C for 25 minutes and washed once with FACS buffer. Cells were resuspended in FACS buffer and blocked with normal mouse IgG (Invitrogen) by adding 100 μl 1:1000 diluted normal mouse lgG to each tube and incubated on ice for 10 min. Wash cells with FACS buffer and re-suspend in 100 μl FACs buffer. The cells were then stained with phycoerythrin (PE)-labeled streptavidin (BD Pharmingen, San Diego, CA) and allophycocyanin (APC)-labeled CD3 (eBiocience, San Diego, CA). 1.0 μl PE and APC were each added to tube 2 and 3.

Flow cytometry acquisition was performed with a BD FacsCalibur (BD Biosciences), and analysis was performed with FlowJo (Treestar, Inc. Ashland, OR).

### Example 8. Isolation of Peripheral Blood Mononuclear Cells (PBMCs)

Whole blood was collected from individual or mixed donors (depending on the amount of blood required) in 10 mL heparin vacutainers (Becton Dickinson). Approximately 10 ml of whole anti-coagulated blood was mixed with sterile phosphate buffered saline (PBS pH 7/4, is without Ca2+/Mg2+).) buffer to achieve a final volume of 20 ml in a 50 ml conical centrifuge tube. 15 mL of Ficoll-Paque PLUS® (GE Healthcare, Catalog No. 17-1440-03) was provided in a sterile 50 mL conical centrifuge tubes and the 20 mL volume of blood/PBS was layered onto the surface of the Ficoll® and centrifuged at 400×g for 30-40 minutes at room temperature. The layer of cells containing peripheral blood mononuclear cells (PBMCs) at the plasma/Ficoll interface was removed carefully. PBMCs were washed twice with PBS in a total volume of 40 ml and centrifuged at 200×g for 10 minutes at room temperature and cells counted with a hemocytometer.

If washed PBMCs were used immediately, the cells were washed once with CAR-T media. CAR-T media is AIM V-AlbuMAX® media (commercially available as catalog Number 31035025 from ThermoFisher Scientific) supplemented with 5% AB serum and 1.25 ug/mL amphotericin B, 100 U/mL penicillin, and 100 μg/mL streptomycin.

If washed PBMCs were not used immediately, the cells were resuspended, washed and transferred to insulated vials and refrigerated at −80 °C for 24 hours before storing in liquid nitrogen.

### Example 9. Activation of PBMCs

PBMCs were prepared in substantial accordance with the teaching of Example _ above. If freshly isolated PBMC were used, isolated cells (washed with 1×PBS (pH7.4), no Ca²⁺/Mg²⁺) are washed once in CAR-T media at a concentration of 1×10⁶ cells/mL. The cells were resuspended to a final concentration of 1×10⁶ cells/mL in CAR-T medium with 300 IU/mL huIL2 (Invitrogen). If frozen PBMC's were used, the cells were thawed and resuspended in 9 mL of pre-warmed (37 °C) cDMEM media (Life Technologies) in the presence of 10% FBS, 100 u/mL penicillin, and 100 μg/mL streptomycin to a concentration of 1×10⁶ cells/mL. The cells were pelleted by centrifugation 300×g for 5 min and washed once in CAR-T media and resuspended to a final concentration of 1×10⁶ cells/mL in CAR-T medium with 300 IU/mL huIL-2.

Anti-human CD28 and CD3 antibody-conjugated magnetic beads (Invitrogen) were washed three times with 1 mL of sterile PBS (pH7.4) using magnetic rack to isolate the beads from the solution and resuspended in CAR-T media supplemented with 300 IU/mL huIL-2 to a final concentration of 4×10⁷ beads/mL.

PBMC cells and the CD28 and CD3 antibody-conjugated magnetic beads were mixed at a 1:1 bead-to-cell ratio.

Aliquots were transferred to single wells of a 12-well low-attachment, or non-treated cell culture plate and incubated in the presence of CO₂ for 24 hours prior to viral transduction.

### Example 10. Lentiviral CAR Expression Vector Construction

A CAR expression cassette comprising nucleic acid sequences encoding the extracellular sequence of an anti-CD19 single chain antibody (ScFv sequence of FMC63 as described in Nicholson, et al. (1997) Construction and characterization of afunctional CD19 specific single chain Fv fragment for immunotherapy of B lineage leukaemia and lymphoma, Molecular Immunology 34:1157-1165 linked to CD8 hinge, 4-1-BB costimulatory domain, and CD3 zeta activation domain was prepared. The CAR expression cassette was cloned into the lentiviral plasmid Lenti CMV-MCS-EF1a-puro (Alstem, Richmond, Calif) to prepare plasmid ST1165. These plasmids were transfected into HEK293 cells to generate recombinant lentivirus which were subsequently used to transduce primary human T cells, isolated from whole blood.

### Example 11. Lentiviral CAR Plus IL-10 Expression Vector Construction

To prepare CAR-T cells which express both the CAR and hIL-10, a chimeric antigen receptor (CAR) lentiviral plasmid PMC 303 was prepared in substantial accordance with the teaching of Example 10 above wherein a nucleic acid sequence was inserted downstream of the CAR coding sequence with an intervening EF1a core promoter sequence to facilitate expression of the IL-10 coding sequence.

### Example 12. Generation of Lentiviral Particles

For the production of lentiviral particles, three components are generally required: 1) a lentiviral vector, 2) packaging vectors containing all necessary viral structural proteins, 3) an envelope vector expressing Vesicular Stomatitis Virus (VSV) glycoprotein (G). Lentiviral packaging was achieved using the SuperLenti™ Lentivirus Packaging System (commercially available from Alstem LLC, 2600 Hilltop Drive, Building B, STE C328, Richmond, CA 94806) in substantial accordance with the manufacturer's instructions.

### Example 13. T-Cell Transduction and Expansion

Activated PBMCs prepared in accordance with Examples 8 and 9 herein were incubated for 24 hours at 37 °C, 5% CO₂. The activated PBMCs were transduced with the high-titer lentiviral particles prepared in accordance with Example 12 herein at a multiplicity of infection (MOI) of 5. Cells were grown in the presence of 300 IU/mL of human IL-2 for a period of 12-14 days depending on the number of CAR-T cells desired with media being added from time to time to maintain a cell concentration of 1×10⁶ cells/mL. Expression of anti-CD19 CAR's were detected by flow cytometry, using an anti-mouse Fab antibody fragment to detect the anti-CD19 scFv.

### Example 14. Evaluation of Cytoxicity Using xCELLigence RTCA

In vitro, confluence and cytoxicity were assessed by cellular impedance assay using the xCELLigence® real time cell analysis (RTCA) procedure using the RTCA iCELLigence® system and software (commercially available from Acea Biosciences, Inc., 6779 Mesa Ridge Road, #100, San Diego CA 92121) in substantial accordance with the instructions provided by the manufacturer. The xCELLigence system uses an “E-plate” which is a multi-well plate, the bottom of each well providing a surface impregnated with an array of electrodes. As cells proliferate across the surface, the electrical impedance across the electrode array increases. As cells die and lift from the plate causing a reduction in electrical impedance. Thus, by measuring the impedance of electron flow across the array, one is able to measure viability of the cells frequently in real time. The impedance of electron flow caused by the adherent cells is reported as Cell Index (CI), a unitless parameter calculated as:

Cell Index (CI)=(impedence at time point n−impedence in the absence of cells) nominal impedance value

As adherent cells proliferate across the surface of the plate, the CI rises reflecting an increase in electrical impedance. When the CI plateaus, the cells are presumed to be confluent on the plate.

Data demonstrates that the addition of an IL-10 agent to CAR-T cells mediated specific enhancement of CAR-T cytotoxicity in an IL-10 agent dose dependent fashion. In particular, data demonstrates that the significant enhancement of target cell cytotoxicity in the presence of an IL-10 agent. In particular, the enhanced cytotoxic effect of the CAR-T cells against the target neoplastic cells is observed even a very low concentrations of IL-10 (0.1 ng/ml). This data illustrates that administration of IL-10 agents to achieve a serum trough concentration of less than about 0.1 ng/ml, alternatively less than about 0.08 ng/ml, alternatively less than about 0.06 ng/ml, alternatively less than about 0.05 ng/ml, alternatively less than about 0.03 ng/ml, alternatively less than about 0.01 ng/ml would be useful in enhancing the therapeutic effect of (or reducing the toxicity of) a CAR-T cell therapy in human subjects.

### Example 15. Effect of Pre-Treatment with IL-10 on Cytoxicity CAR-T Cells

To evaluate the effect of pre-treatment of IL-10 on the cytoxicity of CAR-T cells, the anti-CD19 CAR-T cells were washed and incubated for 24 hours at 37 C, 5% CO₂ in media (in the absence of IL-2) containing varying concentrations of the IL-10 agent AM0010 at the following concentrations: (a) 1000 ng/ml; (b) 100 ng/ml; (c) 10 ng/ml; (e) 1 ng/ml; (f) no AM0010.

In parallel with the period of incubation of the CAR-T cells, HeLa cells (ATCC CCL-2) stably transfected with CD19 (“CD19/HeLa cells”) per well in triplicate were left to adhere to xCELLigence E-plates (ACEA Bioscience, San Diego CA) with approximately 10,000 cells per well. Cells were allowed to adhere until the CI value plateaued reflecting that the cells had reached confluence (approximately 18-20 hours).

The anti-CD19 CAR-T cells prepared as above were then added to the CD19/HeLa cell plates (in triplicate) a varying Effector:Target (E:T) ratios of anti-CD19 CAR-T cells to CD19/HeLa cells (E:T ratio) at the following concentrations: (a) 100,000 CAR-T Cells (10:1 E:T ratio); (b) 50,000 CAR-T Cells (5:1 E:T ratio); (c) 20,000 CAR-T Cells (2:1 E:T ratio); and (e) 10,000 CAR-T Cells (1:1 E:T ratio).

The IL-10 agent AM0010 was added to each well to maintain the prior incubation levels of IL-10 agents (i.e., 1000 ng/ml, 100 ng/ml, 10 ng/ml, 1 ng/ml and 0 ng/ml) during the course of exposure to the HeLa cells to the anti-CD19 CAR-T cells, with respect to each E:T ratio. Cytotoxicity of the Anti-CD19 CAR-T cells to the HeLa cells is assessed by a reduction of electrical resistance as the CAR-T cells kill the Hela cells which detach from the plate. The electrical resistance data was collected every 2 minutes during the course of the experiment and the data analyzed using the software provided with the iCELLigence® system. The data from each triplicate well was combined and averaged using the same software.

An increase in impedance for a period of approximately 1 hour following the time point of addition of the anti-CD19 CAR-T cells observed was attributed to be a result of the anti-CD19 CAR-T cells adhering to the plate and increasing the impedance as measured by the xCELLigence system. However, from the time point of approximately 1 hour following the addition of the anti-CD19 CAR-T cells, a steady reduction in CI was observed indicating effective killing of the CD19/HeLa cells by the anti-CD19 CAR-T cells and a significant enhanced cytotoxic effect at all levels of the IL-10 agent evaluated. This data demonstrates that the addition of IL-10 enhances the cytotoxicity of CAR-T cells against tumor cells.

The data obtained from the foregoing experiment was replotted as histograms demonstrating the enhanced cytotoxic effect on a culture of 10,000 CD19/HeLa cells by the addition an IL-10 agent (AM0010) at varying concentrations (0 ng/ml, 1 ng/ml, 10 ng/ml, 100 ng/ml and 1000 ng/ml) as indicated in combination with and varying amounts of anti-CD-19 CAR-T cells. The addition of AM0010 enhanced the cytotoxic effect of anti-CD-19 CAR-T cells on CD19/HeLa cells at all ratios of anti-CD-19 CAR-T to CD19/HeLa cells at all tested concentrations of AM-0010.

### Example 16. Treatment with IL-10 Agents Enhances of CAR-T Cells Activation

Additionally, a hallmark of T-cell activation in response to exposure to IL-10 agents is enhanced expression of IFN-gamma. The addition of IL-10 to the treatment resulted in significant upregulation of IFN-gamma production in CAR-T cells in an IL-10 dose dependent manner.

### Example 17. In Vivo Evaluation

A study was conducted to evaluate the effect the combination of an IL-10 agent (AM-0010) with anti-tumor CAR-T cell therapy in an in vivo tumor model of neoplastic disease in mice.

Briefly, cohorts of 5 Female NOD.Cg-Prkdcscid IL2rgtm1Wjl/SzJ (NOD/scid IL2RGnull) mice from Jackson Lab were inoculated intraperitoneally with 0.5×10⁶ Raji-luc cells, a CD19+Raji human Burkitt's lymphoma cell line constructed by engineering the Raji cell line (obtained from ATCC as CCL-86) by transduction with a vector providing the luciferase gene. Expression of the luciferase gene enables enabling bioluminescent imaging to evaluate tumor growth by full body bioluminescence accordance with techniques well known in the art (Chen and Thorne, Practical Methods for Molecular In Vivo Optical Imaging; (2012) Current Protocols in Cytometry 59(1):12.24.1-12.24.11).

CAR-T cells were prepared in substantial accordance with the teaching of Example XXX hereinabove. A summary study design treatment groups and the test agents administered is provided in Table 6 below.

TABLE 6
In Vivo Mouse Raji Tumor Study Design

| | Test Article Administered | | |
|---|---|---|---|
| | T-cells (non-CAR) | CAR-T Cells | AM0010 |
| Group # | (millions) | (millions) | (mg/kg) |
| 1 | 0 | 0 | 0 |
| 10 | 0 | 0 | 0.5 |
| 2 | 5 | 0 | 0 |
| 3 | 5 | 0 | 0.5 |
| 4 | 0 | 5 | 0 |
| 5 | 0 | 5 | 0.5 |
| 6 | 0 | 2.5 | 0 |
| 7 | 0 | 2.5 | 0.5 |

On Study Day 0, 0.5 million Raji-luc tumor cells were administered by intravenous injection in a volume of 100 microliters to each mouse. Mice were imaged on the same day prior to the initiation of therapy.

On Study Day 0, treatment with AM-0010 was initiated. AM0010 was administered daily intraperitoneally on Study Days 1-8 and was switched to subcutaneous administration on Day 9 et seq.

On Study Days 2 and 9, in those animals receiving CAR-T cells, or T-cells (mock), the CAR-T or T cells were administered in accordance with Table 6 in a volume of 100 microliters.

Mice were imaged on Study Days 0, 7, 14, 21, 28 and 35 using an IVIS® Spectrum in vivo imaging system (commercially available from Perkin Elmer, 940 Winter St. Waltham MA 02451) in substantial accordance with the manufacturer's instructions.

As indicated, the cancer progressed rapidly in both groups such that all animals in each group were dead by day 21 of the experiment.

There was an effect of the T-cells and CAR-T cells alone as further tumor growth was essentially arrested. Two of the 5 animals in treatment groups 2 and were dead by day 21 and a third animal in treatment group 3 was dead by day 35.

For groups 4 and 5, the effects of administration of 5 million CAR-T cells in the presence of the IL-10 agent AM0010 demonstrates a marked improvement in tumor reduction in the group treated with IL-10 agent in combination with the CAR-T agent. All animals in each treatment group were alive on Day 35 of the study.

For groups 6 and 7, data demonstrates a marked improvement in tumor reduction in the group treated with IL-10 agent in combination with the CAR-T agent at this lower dose of CAR-T cells compared to the data provided as discussed above. All animals in each treatment group were alive on Day 35 of the study.

The foregoing data demonstrates in an art recognized tumor model of the enhanced anti-tumor effect provided by combining CAR-T cell therapy with the administration of an IL-10 agent.

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

We claim:

1. A pharmaceutical composition comprising CAR-T cells wherein CAR-T cells are produced from a sample of T-cells derived from the patient and transduced in the sample with a vector, the vector comprising a nucleic acid sequence encoding a chimeric antigen receptor (CAR) the nucleic acid sequence being in operable association with one or more control elements to effect transcription and translation of the nucleic acid sequence encoding a chimeric antigen receptor (CAR) in a T-cell to generate a population of T-cells that express the CAR and are cultured ex vivo in the presence of an IL-10 agent.

2. The pharmaceutical composition of claim 1, further comprising a therapeutically effective amount of a pharmaceutical formulation comprising an IL-10 agent.

3. The pharmaceutical composition of claim 2, wherein the IL-10 agent of claim 1 is the same as the IL-10 agent of claim 2.

4. The pharmaceutical composition of claim 2, wherein the IL-10 agent of claim 1 and the IL-10 agent of claim 2 are different.

5. The pharmaceutical composition of claim 1, wherein the IL-10 agent comprises a mixture of a mono-PEGylated IL-10 agent and a diPEGylated IL-10 agent.

6. The pharmaceutical composition of claim 2, wherein the IL-10 agent is sufficient to maintain a serum trough concentration of the IL-10 agent in the subject of at least 0.01 ng/ml over a period of at least 72 hours.

7. The pharmaceutical composition of claim 2, wherein the amount of the IL-10 agent combined with the CAR-T cells is sufficient to maintain a serum trough concentration of the IL-10 agent in the subject of at least 0.05 ng/ml over a period of at least 72 hours.

8. The pharmaceutical composition of claim 2, wherein the amount of the IL-10 agent combined with the CAR-T cells is sufficient to maintain a serum trough concentration of the IL-10 agent in the subject of at least 0.1 ng/ml over a period of at least 72 hours.

9. The pharmaceutical composition of claim 2, wherein the administering of a pharmaceutical formulation comprising the IL-10 agent is sufficient to maintain a serum trough concentration of the IL-10 agent in the subject of at least 0.5 ng/ml over a period of at least 72 hours.

10. The pharmaceutical composition of claim 1, wherein the IL-10 agent is an IL-10 variant derived from hIL-10.

11. The pharmaceutical composition of claim 1, wherein an antigen recognition domain of the CAR is a polypeptide that specifically binds to HER2, MUC1, telomerase, PSA, CEA, VEGF, VEGF-R2, T1, CD19, CD20, CD22, ROR1, mesothelin, CD33/IL3Ra, c-Met, PSMA, Glycolipid F77, FAP, EGFRvIII, GD-2, NY-ESO-1 TCR, MAGE A3 TCR, 5T4, WT1, KG2D ligand, folate receptor (FRa), platelet-derived growth factor receptor A, or Wnt1 antigens.

12. The pharmaceutical composition of claim 1, wherein an antigen recognition domain of the CAR is selected from the group consisting of an anti-CD19 scFv, an anti-PSA scFv, an anti-CD19 scFv, an anti-HER2 scFv, an anti-CEA scFv, an anti-EGFR scFv, an anti-MUC1 scFv, an anti-HER2-neu scFv, an anti-VEGF-R2 scFv, an anti-T1 scFv, an anti-CD22 scFv, an anti-ROR1 scFv, an anti-mesothelin scFv, an anti-CD33/IL3Ra scFv, an anti-c-Met scFv, an anti-PSMA scFv, an anti-Glycolipid F77 scFv, an anti-FAP scFv, an anti-EGFRvIII scFv, an anti-GD-2 scFv, an anti-NY-ESO-1 scFv, an anti-MAGE scFv, an anti-A3 scFv, an anti-5T4 scFv, an anti-WT1 scFv, and an anti-Wnt1 scFv.

13. The pharmaceutical composition of claim 1, wherein an intracellular signaling domain of the CAR is a polypeptide comprising an amino acid sequence derived from the cytoplasmic domain of CD27, CD28, CD137 CD278, CD134, FcεR1γ and β chains, MB1 (Igα) chain, B29 (Igβ) chain, the human CD3 zeta chain, CD3, a syk family tyrosine kinase, a src family tyrosine kinase, CD2, CD5 or CD28.

14. The pharmaceutical composition of claim 1, wherein an intracellular signaling domain of the CAR comprises an amino acid sequence derived from the cytoplasmic domain of CD28, CD137 (4-1BB), CD134 (OX40), Dap10, CD27, CD2, CD5, ICAM-1, LFA-1 (CD11a/CD18), Lck, TNFR-I, TNFR-II, Fas, CD30, or CD40.

15. The pharmaceutical composition of claim 1, further comprising one or more supplemental agents.

16. The pharmaceutical composition of claim 15, wherein the one or more supplemental agents is selected from the group consisting of chemotherapeutic agents, immune checkpoint modulators, IL-2 agents, IL-7 agents, IL-12 agents, IL-15 agents and IL-18 agents.

17. The pharmaceutical composition of claim 15, wherein the one or more supplemental agents is one or more chemotherapeutic agents.

18. The pharmaceutical composition of claim 15, wherein the one or more supplemental agents is one or more immune checkpoint modulators selected from the group consisting of PD1 modulators, PDL1 modulators, CTLA4 modulators, LAG-3 modulators, TIM-3 modulators, ICOS modulators, OX40 modulators, cd-27 modulators, CD-137 modulators, HVEM modulators, CD28 modulators, CD226 modulators, GITR modulators, BTLA modulators, A2A modulators, IDO modulators and VISTA modulators.

19. The pharmaceutical composition of claim 18, wherein the immune checkpoint modulator is an antibody.


SEQUENCE LISTING (63 sequences)

SEQ ID NO: 1 - protein, 11 aa, synthetic construct
  REGION 1..11: note = Synthetic construct
YGRKKRRQRRR

SEQ ID NO: 2 - protein, 12 aa, synthetic construct
  REGION 1..12: note = Synthetic construct
RRQRRTSKLMKR

SEQ ID NO: 3 - protein, 27 aa, synthetic construct
  REGION 1..27: note = Synthetic construct
GWTLNSAGYLLGKINLKALAALAKKIL

SEQ ID NO: 4 - protein, 33 aa, synthetic construct
  REGION 1..33: note = Synthetic construct
KALAWEAKLAKALAKALAKHLAKALAKALKCEA

SEQ ID NO: 5 - protein, 16 aa, synthetic construct
  REGION 1..16: note = Synthetic construct
RQIKIWFQNRRMKWKK

SEQ ID NO: 6 - protein, 11 aa, synthetic construct
  REGION 1..11: note = Synthetic construct
YGRKKRRQRRR

SEQ ID NO: 7 - protein, 9 aa, synthetic construct
  REGION 1..9: note = Synthetic construct
RKKRRQRRR

SEQ ID NO: 8 - protein, 11 aa, synthetic construct
  REGION 1..11: note = Synthetic construct
YGRKKRRQRRR

SEQ ID NO: 9 - protein, 8 aa, synthetic construct
  REGION 1..8: note = Synthetic construct
RKKRRQRR

SEQ ID NO: 10 - protein, 11 aa, synthetic construct
  REGION 1..11: note = Synthetic construct
YARAAARQARA

SEQ ID NO: 11 - protein, 11 aa, synthetic construct
  REGION 1..11: note = Synthetic construct
THRLPRRRRRR

SEQ ID NO: 12 - protein, 11 aa, synthetic construct
  REGION 1..11: note = Synthetic construct
GGRRARRRRRR

SEQ ID NO: 13 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 14 - protein, 161 aa, synthetic construct
  REGION 1..161: note = Synthetic construct
MSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 15 - protein, 161 aa, synthetic construct; identical to SEQ ID NO: 14
  REGION 1..161: note = Synthetic construct
  SITE 1: note = MISC_FEATURE - N-formyl group on methionine

SEQ ID NO: 16 - protein, 100 aa, synthetic construct
  REGION 1..100: note = Synthetic construct
  VARIANT 1..100: note = This sequence may encompass 1-20 GSGGS repeating units
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 17 - protein, 80 aa, synthetic construct
  REGION 1..80: note = Synthetic construct
  VARIANT 1..80: note = This sequence may encompass 1-20 GGGS repeating units
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 18 - protein, 100 aa, synthetic construct; identical to SEQ ID NO: 16
  REGION 1..100: note = Synthetic construct
  VARIANT 1..100: note = This sequence may encompass 1-20 GSGGS repeating units

SEQ ID NO: 19 - protein, 1200 aa, synthetic construct
  REGION 1..1200: note = Synthetic construct
  VARIANT 1..20: note = This region may encompass up to 20 residues
  VARIANT 21..40: note = This region may encompass up to 20 residues
  VARIANT 41..60: note = This region may encompass up to 20 residues
  VARIANT 61..80: note = This region may encompass up to 20 residues
  VARIANT 81..100: note = This region may encompass up to 20 residues
  VARIANT 101..120: note = This region may encompass up to 20 residues
  VARIANT 121..140: note = This region may encompass up to 20 residues
  VARIANT 141..160: note = This region may encompass up to 20 residues
  VARIANT 161..180: note = This region may encompass up to 20 residues
  VARIANT 181..200: note = This region may encompass up to 20 residues
  VARIANT 201..220: note = This region may encompass up to 20 residues
  VARIANT 221..240: note = This region may encompass up to 20 residues
  VARIANT 241..260: note = This region may encompass up to 20 residues
  VARIANT 261..280: note = This region may encompass up to 20 residues
  VARIANT 281..300: note = This region may encompass up to 20 residues
  VARIANT 301..320: note = This region may encompass up to 20 residues
  VARIANT 321..340: note = This region may encompass up to 20 residues
  VARIANT 341..360: note = This region may encompass up to 20 residues
  VARIANT 361..380: note = This region may encompass up to 20 residues
  VARIANT 381..400: note = This region may encompass up to 20 residues
  VARIANT 401..420: note = This region may encompass up to 20 residues
  VARIANT 421..440: note = This region may encompass up to 20 residues
  VARIANT 441..460: note = This region may encompass up to 20 residues
  VARIANT 461..480: note = This region may encompass up to 20 residues
  VARIANT 481..500: note = This region may encompass up to 20 residues
  VARIANT 501..520: note = This region may encompass up to 20 residues
  VARIANT 521..540: note = This region may encompass up to 20 residues
  VARIANT 541..560: note = This region may encompass up to 20 residues
  VARIANT 561..580: note = This region may encompass up to 20 residues
  VARIANT 581..600: note = This region may encompass up to 20 residues
  VARIANT 601..620: note = This region may encompass up to 20 residues
  VARIANT 621..640: note = This region may encompass up to 20 residues
  VARIANT 641..660: note = This region may encompass up to 20 residues
  VARIANT 661..680: note = This region may encompass up to 20 residues
  VARIANT 681..700: note = This region may encompass up to 20 residues
  VARIANT 701..720: note = This region may encompass up to 20 residues
  VARIANT 721..740: note = This region may encompass up to 20 residues
  VARIANT 741..760: note = This region may encompass up to 20 residues
  VARIANT 761..780: note = This region may encompass up to 20 residues
  VARIANT 781..800: note = This region may encompass up to 20 residues
  VARIANT 801..820: note = This region may encompass up to 20 residues
  VARIANT 821..840: note = This region may encompass up to 20 residues
  VARIANT 841..860: note = This region may encompass up to 20 residues
  VARIANT 861..880: note = This region may encompass up to 20 residues
  VARIANT 881..900: note = This region may encompass up to 20 residues
  VARIANT 901..920: note = This region may encompass up to 20 residues
  VARIANT 921..940: note = This region may encompass up to 20 residues
  VARIANT 941..960: note = This region may encompass up to 20 residues
  VARIANT 961..980: note = This region may encompass up to 20 residues
  VARIANT 981..1000: note = This region may encompass up to 20 residues
  VARIANT 1001..1020: note = This region may encompass up to 20 residues
  VARIANT 1021..1040: note = This region may encompass up to 20 residues
  VARIANT 1041..1060: note = This region may encompass up to 20 residues
  VARIANT 1061..1080: note = This region may encompass up to 20 residues
  VARIANT 1081..1100: note = This region may encompass up to 20 residues
  VARIANT 1101..1120: note = This region may encompass up to 20 residues
  VARIANT 1121..1140: note = This region may encompass up to 20 residues
  VARIANT 1141..1160: note = This region may encompass up to 20 residues
  VARIANT 1161..1180: note = This region may encompass up to 20 residues
  VARIANT 1181..1200: note = This region may encompass up to 20 residues
  VARIANT 1..1200: note = This sequence may encompass 1-20 GmSoGm repeating units, wherein m and o independently represent 1-20
GGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGG

SEQ ID NO: 20 - protein, 2000 aa, synthetic construct
  REGION 1..2000: note = Synthetic construct
  VARIANT 1..20: note = This region may encompass up to 20 residues
  VARIANT 21..40: note = This region may encompass up to 20 residues
  VARIANT 41..60: note = This region may encompass up to 20 residues
  VARIANT 61..80: note = This region may encompass up to 20 residues
  VARIANT 81..100: note = This region may encompass up to 20 residues
  VARIANT 101..120: note = This region may encompass up to 20 residues
  VARIANT 121..140: note = This region may encompass up to 20 residues
  VARIANT 141..160: note = This region may encompass up to 20 residues
  VARIANT 161..180: note = This region may encompass up to 20 residues
  VARIANT 181..200: note = This region may encompass up to 20 residues
  VARIANT 201..220: note = This region may encompass up to 20 residues
  VARIANT 221..240: note = This region may encompass up to 20 residues
  VARIANT 241..260: note = This region may encompass up to 20 residues
  VARIANT 261..280: note = This region may encompass up to 20 residues
  VARIANT 281..300: note = This region may encompass up to 20 residues
  VARIANT 301..320: note = This region may encompass up to 20 residues
  VARIANT 321..340: note = This region may encompass up to 20 residues
  VARIANT 341..360: note = This region may encompass up to 20 residues
  VARIANT 361..380: note = This region may encompass up to 20 residues
  VARIANT 381..400: note = This region may encompass up to 20 residues
  VARIANT 401..420: note = This region may encompass up to 20 residues
  VARIANT 421..440: note = This region may encompass up to 20 residues
  VARIANT 441..460: note = This region may encompass up to 20 residues
  VARIANT 461..480: note = This region may encompass up to 20 residues
  VARIANT 481..500: note = This region may encompass up to 20 residues
  VARIANT 501..520: note = This region may encompass up to 20 residues
  VARIANT 521..540: note = This region may encompass up to 20 residues
  VARIANT 541..560: note = This region may encompass up to 20 residues
  VARIANT 561..580: note = This region may encompass up to 20 residues
  VARIANT 581..600: note = This region may encompass up to 20 residues
  VARIANT 601..620: note = This region may encompass up to 20 residues
  VARIANT 621..640: note = This region may encompass up to 20 residues
  VARIANT 641..660: note = This region may encompass up to 20 residues
  VARIANT 661..680: note = This region may encompass up to 20 residues
  VARIANT 681..700: note = This region may encompass up to 20 residues
  VARIANT 701..720: note = This region may encompass up to 20 residues
  VARIANT 721..740: note = This region may encompass up to 20 residues
  VARIANT 741..760: note = This region may encompass up to 20 residues
  VARIANT 761..780: note = This region may encompass up to 20 residues
  VARIANT 781..800: note = This region may encompass up to 20 residues
  VARIANT 801..820: note = This region may encompass up to 20 residues
  VARIANT 821..840: note = This region may encompass up to 20 residues
  VARIANT 841..860: note = This region may encompass up to 20 residues
  VARIANT 861..880: note = This region may encompass up to 20 residues
  VARIANT 881..900: note = This region may encompass up to 20 residues
  VARIANT 901..920: note = This region may encompass up to 20 residues
  VARIANT 921..940: note = This region may encompass up to 20 residues
  VARIANT 941..960: note = This region may encompass up to 20 residues
  VARIANT 961..980: note = This region may encompass up to 20 residues
  VARIANT 981..1000: note = This region may encompass up to 20 residues
  VARIANT 1001..1020: note = This region may encompass up to 20 residues
  VARIANT 1021..1040: note = This region may encompass up to 20 residues
  VARIANT 1041..1060: note = This region may encompass up to 20 residues
  VARIANT 1061..1080: note = This region may encompass up to 20 residues
  VARIANT 1081..1100: note = This region may encompass up to 20 residues
  VARIANT 1101..1120: note = This region may encompass up to 20 residues
  VARIANT 1121..1140: note = This region may encompass up to 20 residues
  VARIANT 1141..1160: note = This region may encompass up to 20 residues
  VARIANT 1161..1180: note = This region may encompass up to 20 residues
  VARIANT 1181..1200: note = This region may encompass up to 20 residues
  VARIANT 1201..1220: note = This region may encompass up to 20 residues
  VARIANT 1221..1240: note = This region may encompass up to 20 residues
  VARIANT 1241..1260: note = This region may encompass up to 20 residues
  VARIANT 1261..1280: note = This region may encompass up to 20 residues
  VARIANT 1281..1300: note = This region may encompass up to 20 residues
  VARIANT 1301..1320: note = This region may encompass up to 20 residues
  VARIANT 1321..1340: note = This region may encompass up to 20 residues
  VARIANT 1341..1360: note = This region may encompass up to 20 residues
  VARIANT 1361..1380: note = This region may encompass up to 20 residues
  VARIANT 1381..1400: note = This region may encompass up to 20 residues
  VARIANT 1401..1420: note = This region may encompass up to 20 residues
  VARIANT 1421..1440: note = This region may encompass up to 20 residues
  VARIANT 1441..1460: note = This region may encompass up to 20 residues
  VARIANT 1461..1480: note = This region may encompass up to 20 residues
  VARIANT 1481..1500: note = This region may encompass up to 20 residues
  VARIANT 1501..1520: note = This region may encompass up to 20 residues
  VARIANT 1521..1540: note = This region may encompass up to 20 residues
  VARIANT 1541..1560: note = This region may encompass up to 20 residues
  VARIANT 1561..1580: note = This region may encompass up to 20 residues
  VARIANT 1581..1600: note = This region may encompass up to 20 residues
  VARIANT 1601..1620: note = This region may encompass up to 20 residues
  VARIANT 1621..1640: note = This region may encompass up to 20 residues
  VARIANT 1641..1660: note = This region may encompass up to 20 residues
  VARIANT 1661..1680: note = This region may encompass up to 20 residues
  VARIANT 1681..1700: note = This region may encompass up to 20 residues
  VARIANT 1701..1720: note = This region may encompass up to 20 residues
  VARIANT 1721..1740: note = This region may encompass up to 20 residues
  VARIANT 1741..1760: note = This region may encompass up to 20 residues
  VARIANT 1761..1780: note = This region may encompass up to 20 residues
  VARIANT 1781..1800: note = This region may encompass up to 20 residues
  VARIANT 1801..1820: note = This region may encompass up to 20 residues
  VARIANT 1821..1840: note = This region may encompass up to 20 residues
  VARIANT 1841..1860: note = This region may encompass up to 20 residues
  VARIANT 1861..1880: note = This region may encompass up to 20 residues
  VARIANT 1881..1900: note = This region may encompass up to 20 residues
  VARIANT 1901..1920: note = This region may encompass up to 20 residues
  VARIANT 1921..1940: note = This region may encompass up to 20 residues
  VARIANT 1941..1960: note = This region may encompass up to 20 residues
  VARIANT 1961..1980: note = This region may encompass up to 20 residues
  VARIANT 1981..2000: note = This region may encompass up to 20 residues
  VARIANT 1..2000: note = This sequence may encompass 1-20 GmSoGmSoGm repeating units, wherein m and o independently represent 1-20
GGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGGGGGGGGGGGGGGGGGGGGGGSSSSSSSSSSSSSSSSSSSSGGGGGGGGGGGGGGGGGGGGSSSSSSSSSSSSSSSSSSSSGGGGGGGGGGGGGGGGGGGG

SEQ ID NO: 21 - protein, 480 aa, synthetic construct
  REGION 1..480: note = Synthetic construct
  VARIANT 5..24: note = This region may encompass up to 20 residues
  VARIANT 29..48: note = This region may encompass up to 20 residues
  VARIANT 53..72: note = This region may encompass up to 20 residues
  VARIANT 77..96: note = This region may encompass up to 20 residues
  VARIANT 101..120: note = This region may encompass up to 20 residues
  VARIANT 125..144: note = This region may encompass up to 20 residues
  VARIANT 149..168: note = This region may encompass up to 20 residues
  VARIANT 173..192: note = This region may encompass up to 20 residues
  VARIANT 197..216: note = This region may encompass up to 20 residues
  VARIANT 221..240: note = This region may encompass up to 20 residues
  VARIANT 245..264: note = This region may encompass up to 20 residues
  VARIANT 269..288: note = This region may encompass up to 20 residues
  VARIANT 293..312: note = This region may encompass up to 20 residues
  VARIANT 317..336: note = This region may encompass up to 20 residues
  VARIANT 341..360: note = This region may encompass up to 20 residues
  VARIANT 365..384: note = This region may encompass up to 20 residues
  VARIANT 389..408: note = This region may encompass up to 20 residues
  VARIANT 413..432: note = This region may encompass up to 20 residues
  VARIANT 437..456: note = This region may encompass up to 20 residues
  VARIANT 461..480: note = This region may encompass up to 20 residues
  VARIANT 1..480: note = This sequence may encompass 1-20 GSGGSm repeating units, wherein m = 1-20
GSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSSGSGGSSSSSSSSSSSSSSSSSSSS

SEQ ID NO: 22 - protein, 480 aa, synthetic construct
  REGION 1..480: note = Synthetic construct
  VARIANT 4..23: note = This region may encompass up to 20 residues
  VARIANT 28..47: note = This region may encompass up to 20 residues
  VARIANT 52..71: note = This region may encompass up to 20 residues
  VARIANT 76..95: note = This region may encompass up to 20 residues
  VARIANT 100..119: note = This region may encompass up to 20 residues
  VARIANT 124..143: note = This region may encompass up to 20 residues
  VARIANT 148..167: note = This region may encompass up to 20 residues
  VARIANT 172..191: note = This region may encompass up to 20 residues
  VARIANT 196..215: note = This region may encompass up to 20 residues
  VARIANT 220..239: note = This region may encompass up to 20 residues
  VARIANT 244..263: note = This region may encompass up to 20 residues
  VARIANT 268..287: note = This region may encompass up to 20 residues
  VARIANT 292..311: note = This region may encompass up to 20 residues
  VARIANT 316..335: note = This region may encompass up to 20 residues
  VARIANT 340..359: note = This region may encompass up to 20 residues
  VARIANT 364..383: note = This region may encompass up to 20 residues
  VARIANT 388..407: note = This region may encompass up to 20 residues
  VARIANT 412..431: note = This region may encompass up to 20 residues
  VARIANT 436..455: note = This region may encompass up to 20 residues
  VARIANT 460..479: note = This region may encompass up to 20 residues
  VARIANT 1..480: note = This sequence may encompass 1-20 GSGSmG repeating units, wherein m = 1-20
GSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSGGSGSSSSSSSSSSSSSSSSSSSSG

SEQ ID NO: 23 - protein, 460 aa, synthetic construct
  REGION 1..460: note = Synthetic construct
  VARIANT 4..23: note = This region may encompass up to 20 residues
  VARIANT 27..46: note = This region may encompass up to 20 residues
  VARIANT 50..69: note = This region may encompass up to 20 residues
  VARIANT 73..92: note = This region may encompass up to 20 residues
  VARIANT 96..115: note = This region may encompass up to 20 residues
  VARIANT 119..138: note = This region may encompass up to 20 residues
  VARIANT 142..161: note = This region may encompass up to 20 residues
  VARIANT 165..184: note = This region may encompass up to 20 residues
  VARIANT 188..207: note = This region may encompass up to 20 residues
  VARIANT 211..230: note = This region may encompass up to 20 residues
  VARIANT 234..253: note = This region may encompass up to 20 residues
  VARIANT 257..276: note = This region may encompass up to 20 residues
  VARIANT 280..299: note = This region may encompass up to 20 residues
  VARIANT 303..322: note = This region may encompass up to 20 residues
  VARIANT 326..345: note = This region may encompass up to 20 residues
  VARIANT 349..368: note = This region may encompass up to 20 residues
  VARIANT 372..391: note = This region may encompass up to 20 residues
  VARIANT 395..414: note = This region may encompass up to 20 residues
  VARIANT 418..437: note = This region may encompass up to 20 residues
  VARIANT 441..460: note = This region may encompass up to 20 residues
  VARIANT 1..460: note = This sequence may encompass 1-20 GGGSm repeating units, wherein m = 1-20
GGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSSGGGSSSSSSSSSSSSSSSSSSSS

SEQ ID NO: 24 - protein, 4 aa, synthetic construct
  REGION 1..4: note = Synthetic construct
GGSG

SEQ ID NO: 25 - protein, 5 aa, synthetic construct
  REGION 1..5: note = Synthetic construct
GGSGG

SEQ ID NO: 26 - protein, 5 aa, synthetic construct
  REGION 1..5: note = Synthetic construct
GSGSG

SEQ ID NO: 27 - protein, 5 aa, synthetic construct
  REGION 1..5: note = Synthetic construct
GSGGG

SEQ ID NO: 28 - protein, 5 aa, synthetic construct
  REGION 1..5: note = Synthetic construct
GGGSG

SEQ ID NO: 29 - protein, 5 aa, synthetic construct
  REGION 1..5: note = Synthetic construct
GSSSG

SEQ ID NO: 30 - protein, 250 aa, synthetic construct
  REGION 1..250: note = Synthetic construct
  VARIANT 1..250: note = This sequence may encompass 1-50 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 31 - protein, 4 aa, synthetic construct
  REGION 1..4: note = Synthetic construct
GGGS

SEQ ID NO: 32 - protein, 5 aa, synthetic construct
  REGION 1..5: note = Synthetic construct
GGGGS

SEQ ID NO: 33 - protein, 4 aa, synthetic construct
  REGION 1..4: note = Synthetic construct
GGSG

SEQ ID NO: 34 - protein, 5 aa, synthetic construct
  REGION 1..5: note = Synthetic construct
GGSGG

SEQ ID NO: 35 - protein, 5 aa, synthetic construct
  REGION 1..5: note = Synthetic construct
GSGSG

SEQ ID NO: 36 - protein, 5 aa, synthetic construct
  REGION 1..5: note = Synthetic construct
GSGGG

SEQ ID NO: 37 - protein, 5 aa, synthetic construct
  REGION 1..5: note = Synthetic construct
GGGSG

SEQ ID NO: 38 - protein, 5 aa, synthetic construct
  REGION 1..5: note = Synthetic construct
GSSSG

SEQ ID NO: 39 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 40 - protein, 143 aa, Homo sapiens
MRISKPHLRSISIQCYLCLLLNSHFLTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 41 - protein, 133 aa, Homo sapiens
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 42 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 43 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 44 - protein, 133 aa, Homo sapiens [sequence omitted]

SEQ ID NO: 45 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 46 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 47 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 48 - protein, 157 aa, Homo sapiens
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 49 - protein, 189 aa, Homo sapiens
MAAEPVEDNCINFVAMKFIDNTLYFIENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 50 - protein, 11 aa, synthetic construct
  REGION 1..11: note = Synthetic construct
GGNSIGSYSVH

SEQ ID NO: 51 - protein, 7 aa, synthetic construct
  REGION 1..7: note = Synthetic construct
DDSDRPS

SEQ ID NO: 52 - protein, 10 aa, synthetic construct
  REGION 1..10: note = Synthetic construct
QVWDTSSYWV

SEQ ID NO: 53 - protein, 10 aa, synthetic construct
  REGION 1..10: note = Synthetic construct
GFTFSSYAMS

SEQ ID NO: 54 - protein, 17 aa, synthetic construct
  REGION 1..17: note = Synthetic construct
DISGGGGTTYYADSVKG

SEQ ID NO: 55 - protein, 10 aa, synthetic construct
  REGION 1..10: note = Synthetic construct
SGTVVTDFDY

SEQ ID NO: 56 - protein, 107 aa, synthetic construct
  REGION 1..107: note = Synthetic construct
SYVLTQPPSVSVAPGQTARVTCGGNSIGSYSVHWYQQKPGQAPVLVVYDDSDRPSGIPERFSGSNSGNTAALTISRVEAGDEADYYCQVWDTSSYWVFGGGTKLTVL

SEQ ID NO: 57 - protein, 119 aa, synthetic construct
  REGION 1..119: note = Synthetic construct
EVQLLESGGGLVQPGGSLRLSCPASGFTFSSYAMSWVRQAPGKGLGWVSDISGGGGTTYYADSVKGRFTISRDNSKNTLYLQMNSLRGEDTAVYYCAKSGTVVTDFDYWGQGTLVTVSS

SEQ ID NO: 58 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 59 - protein, 150 aa, Homo sapiens [sequence omitted]

SEQ ID NO: 60 - protein, 446 aa, synthetic construct
  REGION 1..446: note = Synthetic construct
EVQLLESGGGLVQPGGSLRLSCPASGFTFSSYAMSWVRQAPGKGLGWVSDISGGGGTTYYADSVKGRFTISRDNSKNTLYLQMNSLRGEDTAVYYCAKSGTVVTDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 61 - protein, 213 aa, synthetic construct
  REGION 1..213: note = Synthetic construct
SYVLTQPPSVSVAPGQTARVTCGGNSIGSYSVHWYQQKPGQAPVLVVYDDSDRPSGIPERFSGSNSGNTAALTISRVEAGDEADYYCQVWDTSSYWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 62 - DNA, 1338 bp, synthetic construct
  misc_feature 1..1338: note = Synthetic construct
  source 1..1338: mol_type = other DNA
gaggtccagctcctggaatccgggggcggtctggtccagccgggcggctcgctccgcctgtcctgcccggcgagcggcttcaccttctcctcctacgccatgtcctgggtgaggcaggcccccggcaagggcctcggctgggtcagcgacatctccggcggcggcggcaccacgtactacgcggactcggtgaagggccggttcacgatctcccgggacaactccaagaacaccctgtacctgcagatgaactcactgcggggcgaggacacggcggtgtattactgcgccaagtccggaacggttgtgactgatttcgactactggggccagggcaccctggtgaccgtgtccagcgcctccaccaagggccccagcgtgttccccctggcgccgtgctcgcggagcaccagcgagtccaccgccgcgctcggttgcctcgtcaaggactacttccccgagccggtcacagtgtcatggaactccggcgcgctgacgagcggcgtgcacaccttcccggccgtgctccagtccagcggcctgtacagcctcagtagcgtcgtgacggtgccctcgtcgtcgctgggcacgaagacctacacctgcaacgtggaccacaagccgtccaacaccaaggtcgataagcgagtggagagcaagtacggccccccgtgccccccctgcccggccccggagttcctgggtggcccctccgtgttcctcttccccccgaagcccaaagacaccctcatgatcagccggacgccggaggtcacgtgcgtcgtcgtggacgtgagccaggaagacccggaggtccagttcaactggtacgtggacggcgtcgaggtgcataacgccaagaccaagcctcgcgaggaacagttcaactccacttaccgcgtcgtgtccgtcctcaccgtcctgcaccaggactggctcaacgggaaggaatacaagtgcaaggtctcgaacaagggcctgccgtcgtccatcgagaagaccatcagcaaggccaagggccagccgcgggagccccaggtctacaccctccccccctcccaggaagagatgacgaagaaccaggtgagcctgacgtgcctcgtgaaggggttctacccctccgacatcgcagtcgagtgggagagcaacggccagccggagaacaactacaagacgacccccccggtgctggacagcgacgggtccttcttcctctactcgcgtctcacagtcgacaagtcgcgctggcaggagggcaacgtcttctcgtgctccgtgatgcacgaggccctgcacaaccactacacccagaagtcgctgtccctgtccctgggcaag

SEQ ID NO: 63 - DNA, 639 bp, synthetic construct
  misc_feature 1..639: note = Synthetic construct
  source 1..639: mol_type = other DNA
agctacgtgctgacccagccgccctcggtgtcggtcgccccgggccagacggcacgtgtgacctgcggcggtaacagcatcggctcctactcggtccactggtatcagcagaagccggggcaggccccggtcctggtggtctacgacgacagcgaccgcccgtccggcatccccgaacgcttcagcggctcaaacagcgggaacaccgcggccctgacgatctcgcgcgtcgaggcgggggacgaagccgattactactgccaggtctgggacacctcgagttactgggtgttcggcgggggcacgaagctgaccgtcctcggccagccgaaggccgccccctcagtaaccctgttccccccgtcctcggaggagttgcaggcgaacaaggcgacgctggtgtgcttgatctcggacttctaccccggagcggtgacggtcgcctggaaggccgactcctccccggtcaaggcgggcgtggagacgaccaccccctccaagcagagcaacaacaagtacgccgcctcgagctacctctcgctgacacccgagcagtggaagtcccaccggtcctactcgtgccaggtaacccacgagggctccaccgtcgagaagaccgtggcccccaccgagtgcagc